(19) United States
(12) Patent Application Publication
(10) Pub. No.: US 2025/0160718 A1
(43) Pub. Date: May 22, 2025

(54) SYSTEMS AND METHODS FOR HIGH-DENSITY LONG-TERM INTRACELLULAR ELECTROPHYSIOLOGY

(71) Applicant: President and Fellows of Harvard College, Cambridge, MA (US)

(72) Inventors: Donhee Ham, Cambridge, MA (US); Jun Wang, Cambridge, MA (US); Woo-Bin Jung, Cambridge, MA (US); Hongkun Park, Cambridge, MA (US)

(73) Assignee: President and Fellows of Harvard College, Cambridge, MA (US)

(21) Appl. No.: 18/839,599

(22) PCT Filed: Mar. 24, 2023
(86) PCT No.: PCT/US2023/016198

§ 371 Date: Aug. 19, 2024

Related U.S. Application Data
(60) Provisional application No. 63/323,964, filed on Mar. 25, 2022.

(51) Int. Cl. A61B 5/294

(52) U.S. Cl. CPC A61B 5/294; A61B 2562/0214; A61B 2562/046; A61B 2562/125; A61B 2562/182

(57) ABSTRACT

A device is provided for performing an electrical assessment in a biological environment. The device includes a substrate having an exposed surface having holes. At least one of the holes includes surfaces coated with a conductive material that is in electrical contact with an electrode. The device also includes circuitry controllable to apply a stimulus to the conductive material of the hole by applying the stimulus to the electrode in electrical contact with the hole. The hole may be disposed in a well structure that includes a wall surrounding the hole. The well structure may have a dimension such that a cell of interest in the biological environment is able to cover an entirety of a rim of the wall of the well structure. The wall of the well structure may surround one or more of the holes, which may be in electrical contact with a same electrode.

### CROSS REFERENCE TO RELATED APPLICATIONS

The present application claims the benefit of priority of U.S. Provisional Application No. 63/323,964 filed Mar. 25, 2022, entitled “SYSTEMS AND METHODS FOR HIGH-DENSITY LONG-TERM INTRACELLULAR ELECTROPHYSIOLOGY,” the entire contents of which is incorporated by reference herein.

### FIELD OF THE INVENTION

The present disclosure generally relates to systems and methods for performing intracellular electrophysiology study. More specifically, the present disclosure relates to systems and methods for achieving massively parallelized arrays of thousands of electrodes usable to perform intracellular stimulations of neurons and recordings of synaptic signals from the neurons, with the stimulations and recordings spanning durations exceeding one hour and, in some cases, exceeding three hours.

### BACKGROUND

Understanding relationships between the connectivity and functionality of neuronal circuits has been a goal of the neuroscience community. There has been tremendous efforts to develop high-sensitivity recording and stimulation technologies to reach this goal. Conventional technologies for neuronal investigations include a patch-clamp electrode, which has been found to be useful for its ability to perform high-sensitivity intracellular recordings that are able to measure not only propagation of action potentials (“APs”) in neurons but also subthreshold events such as postsynaptic potentials (“PSPs”). Subthreshold events, the recording of which typically is not achievable by extracellular recording techniques, may be important for establishing and/or deciphering a synaptic connection map and/or for determining a connection strength between neurons. (See [1] In Vitro Multi-Functional Microelectrode Array Featuring 59760 Electrodes, 2048 Electrophysiology Channels, Stimulation, Impedance Measurement, and Neurotransmitter Detection Channels, IEEE Journal of Solid-State Circuits, 2017; and [2] High-Density and Large-Scale MEA System Featuring 236,880 Electrodes at 11.72 μm Pitch for Neuronal Network Analysis, IEEE Symposium on VLSI Circuits, 2020. The disclosures of [1] and [2] are incorporated by reference herein in their entireties.) However, it is difficult if not impossible to scale up patch-clamp electrodes to more than 10 such electrodes. Microelectrode arrays (“MEAs”) have been developed that include dense arrangements of tens of thousands of electrodes or more; however, MEAs typically are configured for extracellular recording, which may be blind to dynamic subthreshold events of single neurons. (See [1] and [2].) Therefore, dense and parallel execution of high-sensitivity intracellular recording across a neuronal network remains a technological goal in neurobiological studies.

### SUMMARY OF THE DISCLOSURE

Various nanoelectrode arrays have been developed in order to parallelize intracellular recording; however, these intracellular devices were found to have fidelities that are inferior to those of patch-clamp devices or could not be applied to neuronal networks. (See [3] A. Hai et al., “In-cell recordings by extracellular microelectrodes,” Nature Methods, 2010, 7 (3): 200-2; and [4] R. Liu et al., “Ultra-Sharp Nanowire Arrays Natively Permeate, Record, and Stimulate Intracellular Activity in Neuronal and Cardiac Networks,” Advanced Functional Materials, 2022, 32 (8). The disclosures of [3] and [4] are incorporated by reference herein in their entireties.) In recent years, complementary metal-oxide semiconductor (CMOS) technology has been used to produce integrated nanoelectrodes having sharp profiles with the potential to be useable for parallelizing intracellular recording for neuronal networks. Such nanoelectrodes may be referred to “nanowires” herein. (See [5] International Publication No. WO 2016/112315 for International Patent Application No. PCT/US2016/012685 entitled “NANOWIRE ARRAYS FOR NEUROTECHNOLOGY AND OTHER APPLICATIONS,” the disclosure of which is incorporated by reference herein in its entirety.)

Disclosed herein are examples of a design for an improved electrode structure comprised of an array of multiple holes, also referred to as a “hole array” herein. The holes may be through-holes in an insulative layer, and each hole may provide an opening to a conductive pad at the hole's base or bottom. Also disclosed herein are examples of methods for manufacturing hole arrays and examples of methods for operating hole arrays for high-density long-duration intracellular electrophysiology investigations. The improved electrode design may offer several advantages over conventional electrode designs used for intracellular electrophysiology investigations.

According to some aspects of the present disclosure, an electrode comprised of a hole array may be fabricated using manufacturing processes based on and/or compatible with CMOS technologies. CMOS technologies are widely used for fabricating large-scale integrated circuits with fine linewidths as narrow as one micron (μm) or smaller; therefore, such technologies may permit hole-array fabrication to be less complex, less costly, and less time-consuming, in comparison with other fabrication schemes presently used for producing nanoelectrodes such as nanowires. In some aspects, an electrode comprised of a hole array as disclosed herein may provide lower electrical impedance due in part to a larger surface area for recording a physiological signal, in comparison with electrodes that do no comprise holes or that comprise a single hole. That is, the surface area interfacing or in contact with a neuron may be larger than conventional non-hole or single-hole electrodes. Without wishing to be bound by a particular theory, the larger surface area in a hole-array electrode may be associated with a lower electrical impedance or a higher conductance. When a lower-impedance hole-array electrode is used for electrophysiological recording applications, a higher intracellular coupling rate and a lower attenuation of recorded signals may be achieved.

According to some aspects of the present disclosure, a device is provided for performing electrical assessment in a biological environment. The device may be comprised of a substrate that can be exposed to the biological environment during an investigation. The substrate may have an outer surface on which there are multiple holes. The outer surface may be readily exposed to the biological environment in which the substrate is situated during an investigation. The holes may be hollow, and each hole may contain a conductive material lining interior walls of the hole. Each hole may not be completely filled with a solid material, such that the conducive material lining the interior walls may be surfaces exposed to the biological environment when the device is put into use in an investigation. The holes may be shaped as trenches, indents, or any open volumes forming recesses in the outer surface of the substrate. For example, a sidewall of a hole may be coated with the conductive material such that the sidewall is conductive and, similarly, a base portion or bottom of the hole may be coated with the conductive material such that the bottom is conductive. In some embodiments, platinum black (also referred to herein as “PtB”) may be used as the conductive material coating the interior walls of the holes. For example, PtB may be deposited in the holes of the substrate via cyclic voltammetry techniques. In some embodiments, the deposited PtB coating the interior walls of the holes may have a nano-scale roughness, Ra, that functions to increase conductance and improve electrical characteristics of an electrophysiological recording of an investigation, by increasing the surface area involved in the investigation.

According to some aspects of the present disclosure, multiple holes may be grouped in a sub-array, and conductive material coating the holes of the sub-array may form part of a same electrode that may interface electrically with a biological specimen in a biological environment. In some embodiments, the holes may be disposed on and above a top surface of a conductive structure, such as a conductive pad or a trace disposed in the substrate, and collectively may form an electrode with the conductive structure. For example, a conductive pad may be provided in the substrate and encapsulated with an insulative material covering a top surface of the pad. A sub-array of multiple holes may then be formed in and through the insulative material on top of the pad. The holes may be coated with conductive material, such that the conductive material in the holes may be in physical and electrical contact with the pad and may collectively (with the pad) become part of an electrode. The holes may be formed as trenches, indents, or any openings in the insulative material covering the pad in the substrate. In some embodiments, PtB may serve as at least part of the conductive material and may be added in the openings after formation of the openings, such that the conductive material is in electrical and physical contact with the underlying pad. In some embodiments, a conductive underlayer may be positioned between the PtB and the insulative material (e.g., at sidewalls of the openings) to promote adhesion of the PtB to the insulative material. The conductive underlayer may be comprised of platinum (Pt) and titanium (Ti), such as a multilayer structure of Ti and Pt (“Ti/Pt”).

According to some aspects of the present disclosure, a device may be comprised of multiple electrodes arranged in an electrode array. Each electrode may be addressed independently by circuitry such that an electrode in a biological environment may be stimulated with one or more electrical stimulation signal(s) to, in turn, stimulate the biological environment with the electrical stimulation signal(s). In some embodiments, the circuitry may be at least partially “off chip” or external to the substrate on which the electrodes are formed and may be connected to the electrodes using any suitable interconnection and packaging techniques now known or future developed, such as but not limited to traces, cables, wires, connectors, wire bonds, wafer bonding, soldering. In some embodiments, the circuitry may at least partially “on chip” and may comprise one or more integrated circuits that co-exist on the substrate together with the electrodes. In some embodiments, a single substrate or chip may be comprised of integrated circuitry and pixel circuits that are independently addressable by the integrated circuitry. In some embodiments, each pixel circuit may include one or more electrodes, and each electrode may include one or more holes.

According to some aspects of the present technology, at least one of the devices disclosed herein may be used in an ex vivo biological environment or an in vivo biological environment. The present technology is not limited to applications involving living organisms but may additionally or alternatively be used in chemical environments to investigate, e.g., chemical reactions. In some aspects, at least one of the devices disclosed herein may be comprised of hole-array electrodes and may be used to perform long-term intracellular electrophysiology investigations of cells such as neurons. The term “long-term” may include a duration or period of time of at least 30 minutes, or at least 60 minutes, or at least 90 minutes, or at least 120 minutes, or at least 150 minutes, or at least 180 minutes. That is, stimulation and/or recording of the neurons may take place over a long-term span of time. In some embodiments, the device may be used in an in vivo application for investigating neuron cells. In such an application, the hole-array electrodes of the device may be part of a probe configured to interface with a brain, with external circuitry interconnected to the electrodes via flexible wiring.

Aspects of the technology disclosed herein are directed to methods of operating one or more of the devices disclosed herein for electrophysiological studies of cells while maintaining intracellular coupling over a long period of time. In some aspects, a feedback mechanism may be used to stop stimulation when intracellular coupling is achieved, and to restart stimulation when a measured amplitude representative of the intracellular coupling falls below a predetermined threshold. Compared to a method that involves supplying a constant stimulus (e.g., a constant electrical stimulation signal), some of the methods disclosed herein have been demonstrated to result in a prolonged intracellular coupling of more than one hour (e.g., more than 2 hours, more than 3 hours).

Circuitry that may be useable with one or more device(s) disclosed herein and/or one or more method(s) disclosed herein for current-based stimulation of electrogenic cells, is described in International Publication No. WO 2019/010343 for International Patent Application No. PCT/US2018/040969 entitled “CURRENT-BASED STIMULATORS FOR ELECTROGENIC CELLS AND RELATED METHODS,” the disclosure of which is hereby incorporated by reference in its entirety.

Circuitry that may be useable with one or more device(s) disclosed herein and/or one or more method(s) disclosed herein for analyzing electrogenic cells is described in International Publication. No. WO 2019/089495 for International Patent Application No. PCT/US2018/058081 entitled “ELECTRONIC CIRCUITS FOR ANALYZING ELECTROGENIC CELLS AND RELATED METHODS,” the disclosure of which is hereby incorporated by reference in its entirety.

Circuitry that may be useable with one or more device(s) disclosed herein and/or one or more method(s) disclosed herein for achieving individually addressable integrated circuits and related technologies is described in International Publication. No. WO 2016/112315 for International Patent Application No. PCT/US2016/012685 entitled “NANOWIRE ARRAYS FOR NEUROTECHNOLOGY AND OTHER APPLICATIONS,” the disclosure of which is hereby incorporated by reference in its entirety.

Apparatuses and methods for performing cell mapping via impedance measurements, aspects of which that may be useable with one or more device(s) disclosed herein and/or one or more method(s) disclosed herein, are described in International Publication. No. WO 2021/257701 for International Application No. PCT/US2021/037626 entitled “APPARATUSES FOR CELL MAPPING VIA IMPEDANCE MEASUREMENTS AND METHODS TO OPERATE THE SAME,” the disclosure of which is hereby incorporated by reference in its entirety.

Systems and methods for patterning and performing spatial electrochemical mapping of cells, aspects of which may be useable with one or more device(s) disclosed herein and/or one or more method(s) disclosed herein, are described in International Publication. No. WO 2021/257705 for International Patent Application No. PCT/US2021/037630 entitled “SYSTEMS AND METHODS FOR PATTERNING AND SPATIAL ELECTROCHEMICAL MAPPING OF CELLS,” the disclosure of which is hereby incorporated by reference in its entirety.

According to an aspect of the technology of the present disclosure, a device is provided for performing an electrical assessment in a biological environment. The device may be comprised of: a substrate having an exposed surface comprised of a plurality of holes, at least some of the holes being defined by recessed surfaces coated with a conductive material in electrical contact with electrodes, the electrodes each being in electrical contact with the conductive material of at least one of the holes; and circuitry controllable to apply one or more stimulus signals to the conductive material of each hole by applying the one or more stimulus signals to the electrode in electrical contact with the hole.

In some embodiments of this aspect, the exposed surface may be configured to be exposed to the biological environment.

In some embodiments of this aspect, for each hole of the at least some of the holes, the conductive material may define an interior that is configured to be exposed to the biological environment. In some embodiments, the interiors of the at least some of the holes may be configured to be exposed to the biological environment via a solution or air.

In some embodiments of this aspect, the biological environment may be an in vivo biological environment.

In some embodiments of this aspect, the circuitry may be coupled to the electrode via one or more passivated wires.

In some embodiments of this aspect, the circuitry may be an integrated circuit disposed on the substrate.

In some embodiments of this aspect, the conductive material may be comprised of platinum black (PtB).

In some embodiments of this aspect, the biological environment may be comprised of at least one cell.

In some embodiments of this aspect, the biological environment may be comprised of at least one neuron.

In some embodiments of this aspect, at least two holes of the at least some of the holes are in electrical contact with a same electrode.

In some embodiments of this aspect, the holes may be circular holes having a same diameter.

In some embodiments of this aspect, the holes may be circular holes having a plurality of different diameters.

In some embodiments of this aspect, the holes may be circular holes and may include first holes having a first diameter and second holes having a second diameter different from the first diameter.

In some embodiments of this aspect, each hole of the at least some of the holes may be disposed in a well structure comprised of a wall surrounding the hole such that the wall surrounds at least one hole of the at least some of the holes. The wall may have a height extending above the exposed surface, with the height of the wall corresponding to a depth of the well. In some embodiments, the well structure may have a dimension such that a cell in the biological environment is able to cover an entirety of a rim of the wall of the well structure. In some embodiments, the wall of the well structure may surround at least two holes of the at least some of the holes, with the at least two holes being in electrical contact with a same electrode. In some embodiments, the wall of the well structure may have a square shape or a circular shape. In some embodiments, the holes may be circular holes and may include first holes having a first diameter and second holes having a second diameter different from the first diameter. In some embodiments, the walls of the well structures may be comprised of first walls and second walls, with each of the first walls surrounding a first area and with each of the second walls surrounding a second area larger than the first area.

According to another aspect of the present disclosure, a substrate is provided. The substrate may be comprised of: an exposed surface comprised of a plurality of holes, at least some of the holes being defined by recessed surfaces coated with a conductive material; and integrated circuitry comprised of a plurality of pixel circuits, each of the pixel circuits being in electrical communication with the conductive material of one or more holes of the at least some of the holes.

In some embodiments of this aspect, for each hole of the at least some of the holes, the hole may be defined by at least one conductive sidewall surrounding an interior that is recessed from the exposed surface. In some embodiments, for each hole of the at least some of the holes, the hole may further be defined by a conductive base portion at a base of the interior. In some embodiments, the at least one conductive sidewall and the conductive base portion may be formed of the conductive material. In some embodiments, the interior may be configured to be exposed to a biological environment disposed on the exposed surface. In some embodiments, for each hole of the at least some of the holes, the at least one conductive sidewall may have a surface roughness, Ra, of between 0.1 nm and 100 nm, or between 0.1 nm and 10 nm, or between 1 nm and 10 nm.

In some embodiments of this aspect, the conductive material may be comprised of platinum (Pt). In some embodiments, the conductive material may be comprised of platinum black (PtB).

In some embodiments of this aspect, the exposed surface may be free of the conductive material of the holes.

In some embodiments of this aspect, for each hole of the at least some of the holes, the hole may be defined by an opening at the exposed surface, and the opening may have a width along a direction parallel to the exposed surface of between 0.5 μm and 5 μm, or between 1 μm and 3 μm, or between 1.5 μm and 2.5 μm. In some embodiments, the opening may be circular and the width may be a diameter.

In some embodiments of this aspect, for each hole of the at least some of the holes, the hole may have a depth extending along a direction perpendicular to the exposed surface, and the depth may be between 1 nm and 5 μm, or between 1 nm and 2 μm, or between 0.5 μm and 2 μm.

In some embodiments of this aspect, for each hole of the at least some of the holes, the hole may be defined by an opening at the exposed surface having a width extending parallel to the exposed surface and a depth extending perpendicular to the exposed surface, and an aspect ratio of the depth over the width may be between 0 and 1000:1, or between 0 and 100:1, or between 0 and 10:1, or between 0 and 1:1, or between 0 and 1:10, or between 1:10 and 1:1.

In some embodiments of this aspect, the substrate may further be comprised of a plurality of electrode pads respectively in electrical communication with the pixel circuits of the integrated circuitry. For each of the pixel circuits, the pixel circuit may be in electrical communication with the one or more holes via a corresponding electrode pad of the electrode pads. In some embodiments, each of the electrode pads may be disposed in an insulative material. In some embodiments, for each of the electrode pads, the insulative material may be in contact with the conductive material of the one or more holes corresponding to the corresponding electrode pad. In some embodiments, for each of the electrode pads, the insulative material may substantially surround the one or more holes corresponding to the corresponding electrode pad. In some embodiments, the exposed surface may be comprised of an insulative material. In some embodiments, a top surface of the one or more holes may be aligned with, or above, or below the exposed surface of the substrate. In some embodiments, the insulative material may be comprised of a plurality of recesses extending to the exposed surface of the substrate, and the one or more holes may be comprised of a conductive coating on an inner surface of each recess of one or more of the recesses. In some embodiments, the insulative material may be comprised of a first insulative layer and a second insulative layer. In some embodiments, the electrode pad may be disposed in the first insulative layer, and the second insulative layer may be disposed between the first insulative layer and the exposed surface. In some embodiments, the exposed surface may be a surface of the second insulative layer. In some embodiments, each hole of the one or more holes may be comprised of a conductive base portion disposed on the corresponding electrode pad. In some embodiments, each hole of the one or more holes may be comprised of a conductive base portion in contact with the corresponding electrode pad. In some embodiments, the one or more holes may be comprised of at least two holes, and the at least two holes may be part of a sub-array of holes in contact with the corresponding electrode pad. In some embodiments, the holes of the sub-array may have a center-to-center distance of between 1 μm and 10 μm, or between 2 μm and 10 μm, or between 3 μm and 5 μm. In some embodiments, a lateral distance between a portion of a hole in the sub-array and an edge of the corresponding electrode pad may be less than 5 μm, or less than 3 μm, or less than 2 μm. In some embodiments, the holes of the sub-array may be arranged in columns and rows. In some embodiments, the corresponding electrode pad may have a lateral dimension along a direction parallel to the exposed surface of between 1 μm and 50 μm, or between 1 μm and 20 μm, or between 10 μm and 20 μm. In some embodiments, the substrate may further be comprised of an electrode array having a plurality of electrode pads in electrical communication with some or all of the pixels circuits, with each of the electrode pads having a sub-array of holes disposed thereon. In some embodiments, the electrode pads of the electrode array may have a center-to-center spacing of between 1 μm and 500 μm, or between 1 μm and 200 μm, or between 10 and 20 μm. In some embodiments, adjacent electrode pads of the electrode pads of the electrode array may be separated by a separation distance of between 1 μm and 500 μm, of between 1 μm and 100 μm, or between 1 μm and 10 μm. In some embodiments, the electrode array may be comprised of at least 200 electrodes, or at least 1000 electrodes, or at least 4000 electrodes, or at least 1,000,000 electrodes.

In some embodiments of this aspect, the one or more holes may be disposed in a well structure comprised of a wall surrounding the one or more holes, with the wall having a height extending above the exposed surface, and with the height of the wall corresponding to a depth of the well. In some embodiments, the well structure may have a dimension such that a or interest disposed on the well structure is able to cover an entirety of a rim of the wall of the well structure. In some embodiments, the wall of the well structure may have a square shape or a circular shape. In some embodiments, the holes may be circular holes and may include first holes having a first diameter and second holes having a second diameter different from the first diameter. In some embodiments, the walls of the well structures may be comprised of first walls and second walls, with each of the first walls surrounding a first area and with each of the second walls surrounding a second area larger than the first area.

According to a further aspect of the present disclosure, a device for electrical assessment of a biological environment is provided. The device may be comprised of: an electrode comprising a plurality of trenches, each trench comprising a conductive material defining an interior configured to be exposed to the biological environment; and circuitry coupled to the electrode and controllable to apply one or more stimulus signals to the electrode.

According to another aspect of the present disclosure, an apparatus is provided. The apparatus may be comprised of: an integrated circuit comprised of a plurality of pixel circuits, each pixel circuit including an electrode comprised of one or more holes; and a solution in contact with the interior of at least some of the holes of the pixel circuits.

According to yet another aspect of the present disclosure, a method of manufacturing a semiconductor device on a substrate is provided. The method may be comprised of: forming an integrated circuit on the substrate, the integrated circuit being comprised of an electrode array; forming at least one insulative layer above the electrode array; forming a plurality of trenches in the at least one insulative layer; and coating one or more sidewalls of the trenches with a conductive material to form an array of holes in electrical contact with the electrode array.

In some embodiments of this aspect, for each hole of the array of holes, the conductive material may define an interior that is recessed from an exposed surface of the substrate and that is exposed to an exterior environment of the semiconductor device.

In some embodiments of this aspect, the coating of the conductive material may be comprised of depositing a layer of the conductive material.

In some embodiments of this aspect, the coating of the conductive material may be comprised of depositing the conductive material by cyclic voltammetry.

In some embodiments of this aspect, the conductive material may be comprised of platinum black (PtB).

In some embodiments of this aspect, the method may further be comprised of, prior to the forming of the trenches, forming an array of well structures in the at least one insulative layer, each of the well structures being comprised of a wall height extending above an exposed surface of the substrate, the height of the wall corresponding to a depth of the well. In some embodiments, the well structure may have a dimension such that a cell of interest is able to cover an entirety of a rim of the wall of the well structure. In some embodiments, the forming of the plurality of trenches may be comprised of forming at least one trench in each well structure such that the wall of the well structure surrounds the at least one trench.

According to another aspect of the present disclosure, a method is provided for operating a substrate of any embodiment described herein to perform intracellular recordings of a cell disposed on the exposed surface of the substrate. The method may be comprised of: (A) stimulating the cell using the integrated circuit; (B) measuring an electrical characteristic using the integrated circuit; (C) determining, based on the measured electrical characteristic, whether intracellular coupling is achieved; and (D) stopping the stimulating of the cell upon a determination that intracellular coupling has been achieved.

In some embodiments of this aspect, the method may further be comprised of: (E) subsequent to (D), repeating (B); (F) determining whether an amplitude of the measured electrical characteristic crosses a predetermined threshold; and (G) upon a determination that the amplitude crosses the threshold, repeating (A) through (D).

The foregoing features may be used, separately or together in any combination, in any of the embodiments discussed herein.

### BRIEF DESCRIPTION OF THE DRAWINGS

A skilled artisan will understand that the accompanying drawings are for illustration purposes only. It is to be understood that in some instances various aspects of the present invention may be shown exaggerated or enlarged to facilitate an understanding of the invention. In the drawings, like reference characters may generally refer to like features, which may be functionally similar and/or structurally similar elements, throughout the various figures. The drawings are not necessarily to scale, as emphasis is instead placed on illustrating and teaching principles of the various aspects of the present technology. The drawings are not intended to limit the scope of the claims or the scope of the present disclosure in any way. In the drawings:

FIG. 1a is a schematic diagram of a side view of an apparatus formed in a semiconductor substrate, according to some embodiments of the present technology;

FIG. 1b is a two-dimensional chart plotting a simulated voltage distribution in the apparatus of FIG. 1a, according to some embodiments of the present technology;

FIG. 1c is a chart plotting simulated electric field lines corresponding to the chart shown in FIG. 1b, according to some embodiments of the present technology;

FIG. 2a is a schematic diagram of a side view of an apparatus without a cell present, in accordance with some embodiments of the present technology;

FIG. 2b is a schematic diagram of a side view of the apparatus of FIG. 2a in which a cell is disposed over some electrodes, according to some embodiments of the disclosed technology;

FIG. 2c is a schematic diagram of a side view of the apparatus of FIG. 2a in which a cell is disposed in a region between adjacent electrodes, according to some embodiments of the disclosed technology;

FIG. 3a shows a measured current-distribution heat map of nearest recording electrodes to a single stimulus electrode when no cell is present (left) and when a cell is present (right). Included in FIG. 3a is a graph 303 plotting measured cross-electrode current versus distance from recording pixel to stimulation (stimulus) pixel, for measurements in the presence of cells (open circles) and with no cell present (filled circles);

FIG. 3b shows a fluorescent microscopy image across an array of electrodes. Included in FIG. 3b is a heat map across the array of electrodes generated using a max current value, Iₑ, determined for each pixel location serving as the stimulus electrode. FIG. 3b also shows a select region overlap map, which is an overlay of a select region of the fluorescent microscopy image and a select region of the heat map;

FIG. 4A schematically shows an example of a high-resolution up-scaled mapping using a 3×3 impedance grid, according to some embodiments of the present technology. An inset in FIG. 4A shows an example of the impedance grid is shown in the inset;

FIG. 4B shows a schematic circuit model that may be used to calculate cell-substrate impedance, Zₛ, and transepithelial impedance, Zₜₑ, for an applied AC stimulation voltage, V_{A}, and to measure a cross electrode current, 112, according to some embodiments of the present technology;

FIG. 5a shows a fluorescent microscopy image across an electrode array, and also shows a heat map of a normalized cross-electrode impedance of a cell culture immediately following plating. Included in FIG. 5a is an inset showing an enlarged portion of the fluorescent microscopy image and an inset showing an enlarged portion of the heat map, revealing a decrease in the cross-electrode normalized cell-substrate impedance, Zₛ, for unadhered cells with single-cell resolution;

FIG. 5b shows a fluorescent microscopy image and a cross-electrode impedance map after 24 hours of exposure to a culture. Included in FIG. 5b is an overlay map corresponding to an enlargement of a select region of the fluorescent microscopy image and the cross-electrode impedance map;

FIG. 6a shows a series of normalized impedance maps over time for MDCK cells with a 5 mM EDTA application and a subsequent washout;

FIG. 6b shows a chart that plots mean normalized impedance values over time for different regions of an electrode array in a cell culture;

FIG. 6c show histograms of the normalized impedance values before, during, and after a washout of EDTA across the electrode array;

FIG. 7 shows a series of fluorescent microscopy images and normalized cross-electrode impedance maps of MDCK cells over seven (7) days of culture in vitro (DIV);

FIG. 8a shows a normalized impedance histogram of a control measurement of MDCK cells without addition of tetracycline;

FIG. 8b shows a normalized impedance histogram of MDCK cells over a span of 6 to 7 days of culture in vitro (DIV), with tetracycline added after the 2 DIV measurement.

FIG. 9 shows a series of normalized cross-electrode impedance maps under stimulus signals of different frequencies;

FIG. 10a schematically depicts how to map cells and their adhesion over time via cross-electrode impedance measurements, according to some embodiments of the present technology;

FIG. 10b schematically depicts an arrangement for measuring a vertical field component;

FIG. 11 schematically depicts detachment of dead cells from an electrode array;

FIG. 12 shows a CMOS electrode array that was used with MDCK cells. The left panel shows the electrode pattern or layout of the electrode array; the middle panel shows an image of the electrode array before patterning; and the right panel shows an image of the electrode array after patterning;

FIGS. 13a through 13d show a series of schematic diagrams illustrating arrangements for cell patterning using an electrode array. In the arrangements of FIGS. 13a and 13b, one or more pre-determined patterning voltages may be applied to selected electrodes for patterned removal of a cell by electrochemical gas generation. In the arrangements of FIGS. 13c and 13d, one or more pre-determined patterning currents may be applied to selected electrodes for patterned removal of a cell;

FIG. 14 shows a series of fluorescent microscope images illustrating the process of defining a co-culture via patterning and then plating a second cell type;

FIG. 15 shows a series of schematic diagrams illustrating a heterogeneous cell population; elimination of undesired cells using patterned electrochemical gas generation on select electrodes; and a homogenous culture of desired properties after subsequent cell growth;

FIG. 16 shows normalized cross-electrode impedance maps for a control culture and for cytochalasin B culture before patterning and at different stages after patterning;

FIG. 17a schematically shows a progression of a permeabilization experiment, according to some embodiments of the present technology;

FIG. 17b shows heat maps for HEK 293 cells using EthD-1 (top row of images) and Fluo-4 (bottom row of images);

FIG. 17c averaged intensity results from the heat maps of FIG. 17b for the HEK 293 cells;

FIG. 17d shows averaged intensity results for neurons for the same test conditions as for the HEK 293 cells of FIG. 17b;

FIG. 18A schematically shows a progression of an electroporation experiment in which fluorescence is monitored over time;

FIG. 18B shows an image of a neuron in fluorescence from an applied electroporation signal, and also shows a chart of fluorescence over time;

FIG. 19 shows a series of schematic diagrams illustrating a process for a control and cross-effect delivery using spatial addressing and serial delivery via gas generation, according to some embodiments of the present technology;

FIG. 20a shows a schematic diagram of a configuration for performing cyclic voltammetry measurements using transimpedance amplifiers (TIAs). Included in FIG. 20a is a cyclic-voltammetry plot of measured current as a function of applied voltage;

FIG. 20b shows two spatial maps of current density: one of a max range of electrodes currents, and one of a max range of currents minus the max/min voltage currents;

FIG. 21a shows data plots of electrode voltages, Vₑₗ, corresponding to open-circuit potentials measured over a period of time;

FIG. 21b shows a heat map that illustrates, for one cycle, an overall amplitude (maximum minus the minimum) of open-circuit potential plotted across an array;

FIG. 21c shows a heat map and a data plot illustrating a minimum time of open-circuit potential plotted versus distance from a center of a 13×13 array of electrodes;

FIG. 22a shows a voltage pulse sequence applied to stimulation electrodes in an electrode array. FIG. 22a also shows a series of data plots of electrode current, Iₑₗ, as a function of time;

FIG. 22b shows a cross-electrode impedance heat map using a cross-electrode max current, Imax, over an electrode array area (upper image), and also shows a heat map of a change in electrode current, ΔIₑₗ, over the electrode array area (lower image), for HEK293 cells;

FIG. 23a shows schematic diagrams illustrate three cell parameters: a cell-substrate impedance, Zₛ; a transepithelial impedance, Zₜₑ; and a extracellular redox potential, V_{redox};

FIG. 23b illustrates a scenario where a fluorescent microscope can be paired with a packaged CMOS IC for simultaneous optical and electrical cell measurements, according to some embodiments of the present technology;

FIG. 23c is a colorized fluorescent image of MDCK epithelial cells cultured on top of an electrode array;

FIG. 23d shows a circuit diagram of an example of a pixel circuit for an electrode in an electrode array;

FIGS. 24a and 24b show schematic diagrams illustrating schemes for cell-cell connectivity measurements, in accordance with some embodiments of the present technology;

FIG. 25a. shows a schematic diagram of an arrangement that uses a Pt electrode for measuring impedance, for potentiometric sensing of oxygen, and for extracellular redox monitoring, according to some embodiments of the present technology;

FIG. 25b shows results of multi-parametric measurements in a series of data maps, of which a top row shows cell attachment, a middle row shows cell-cell adhesion, and a bottom row shows metabolic state;

FIG. 25c shows a pair of nuclei fluorescence images: one for after plating (top image) and a magnification of region 1 for comparison (bottom image);

FIG. 25d is a composite map showing overlaid images for cell nuclei and cell attachment, indicating good spatial correspondence with single-cell resolution;

FIG. 26a shows results of a comparison study of electrode impedance for cells cultured at approximately 72 hours with electrodes under three scenarios: low cell density, high cell density, and without cells;

FIG. 26b shows a data plot indicating that PtB lowered Zₜₑ measurements of bare electrodes by about a factor of 5;

FIG. 26c illustrates cell barrier maps versus a reference at different frequencies;

FIG. 26d shows cell density and connectivity maps extracted from nuclei of fluorescence images;

FIG. 26e shows a comparison between Zₜₑ measured without and with a reference at 1.8 kHz;

FIG. 26f shows a comparison between Zₜₑ and Zₛ versus extracted cell density;

FIG. 27a schematically illustrates cross-sectional views of an example of a fabrication process for a nanowire-based electrode device, including a process A for nanowire fabrication, for producing an electrode comprised of an array of nanowires, and a process B for fabricating an opening to a wire-bonding pad;

FIG. 27b schematically illustrates cross-sectional views of an example of a fabrication process for an electrode comprised of a hole array, according to some embodiments of the technology disclosed herein;

FIG. 28a shows a micrograph of an example of a portion of an electrode comprised of an array of sub-arrays, according to some embodiments of the technology disclosed herein;

FIG. 28b shows a micrograph of a sub-array structure prior to deposition of PtB, according to some embodiments of the technology disclosed herein;

FIG. 28c shows a micrograph of a sub-array structure after deposition of PtB, according to some embodiments of the technology disclosed herein;

FIG. 29 shows charts (i) through (iv), which are examples of experimental recording results obtained using electrodes having a sub-array of 3×3 holes, according to some embodiments of the technology disclosed herein. Each of the charts (i) through (iv) shows signal amplitude (in mV or millivolts) as a function of time (in s or seconds);

FIG. 30a shows an image of an array of electrodes produced according to an embodiment of the technology disclosed herein;

FIGS. 30b through 30e illustrate intracellular coupling capabilities of the array of electrodes of FIG. 30a stimulated using a DC current injected to the electrodes, according to some embodiments of the technology disclosed herein, of which FIG. 30b shows signal amplitudes recorded for electrode sites during an investigation; FIG. 30c shows coupling durations recorded for the electrode sites during the investigation; FIG. 30d shows a distribution chart for coupling duration; and FIG. 30e shows a distribution chart for signal amplitude;

FIG. 31 shows an example of feedback-based stimulation, according to some embodiments of the technology disclosed herein;

FIG. 32a schematically depicts a hole array, according to some embodiments of the present technology;

FIG. 32b shows a micrograph of a portion of a hole array comprised of individual holes fabricated according to some embodiments of the present technology;

FIG. 33a schematically shows a well surrounding a relatively larger single hole, according to some embodiments of the present technology, and also shows a micrograph of a plurality of such wells fabricated according to some embodiments of the present technology;

FIG. 33b schematically shows a well surrounding a relatively smaller single hole, according to some embodiments of the present technology, and also shows a micrograph of a plurality of such wells fabricated according to some embodiments of the present technology;

FIG. 33c schematically shows a well surrounding a plurality of holes, according to some embodiments of the present technology, and also shows a micrograph of a plurality of such wells fabricated according to some embodiments of the present technology;

FIG. 34 schematically shows an interface between a cell and circuitry underneath the cell, according to some embodiments of the present technology. Included in FIG. 34 is an inset schematically illustrating a circuit model for the interface;

FIG. 35a schematically illustrates a general process flow for forming a hole, according to some embodiments of the present technology;

FIG. 35b schematically illustrates cross-sectional views of an example of a fabrication process for forming a hole, according to some embodiments of the present technology;

FIG. 36a schematically shows a device that may be used for in vitro or ex vivo applications, according to some embodiments of the technology disclosed herein;

FIG. 36b schematically shows a device that may be used for in vivo applications, according to some embodiments of the technology disclosed herein;

### DETAILED DESCRIPTION

The inventors have recognized and appreciated that there are limitations to conventional techniques for electrophysiological study. As noted above, MEAs may include tens of thousands of electrodes or more, but typical MEAs are configured for extracellular recording and therefore may be blind to subthreshold dynamics of single neurons. (See [1] and [2], cited above.) In-cell nanoelectrode arrays and nanowires have been developed in order to parallelize intracellular recording, as mentioned above, but such techniques are not CMOS-integrated and, as a result, scalability has been limited. (See [3] and [4], cited above.) A CMOS-based nano-wire electrode array has been developed that is able to achieve parallel intracellular recordings from over a thousand of connected neurons (see [5], cited above); however, fabrication of sharp nanoneedles or nanowires remains complex, time consuming, and expensive, thus presenting a high barrier preventing such arrays from being used more widely. Furthermore, for the conventional electrophysiological techniques noted above, intracellular signal coupling has a limited duration of less than about 20 minutes, which may not provide sufficient time to track some neurological events. As a result, a technique that is able to provide long-term intracellular recording is desired.

Disclosed herein are systems and methods for achieving and/or using a new CMOS-compatible electrode structure in which, in lieu of nanowires, a hole array is used that permits a simplified fabrication process. Hole arrays, as disclosed herein, despite being fabricated by more simplified processes, do not compromise intracellular coupling efficiency. That is, the intracellular coupling efficiency of a hole array, embodiments of which are disclosed herein, is comparable to that of conventional nanowires or nanoneedles. Also disclosed herein are new techniques for prolonging intracellular coupling.

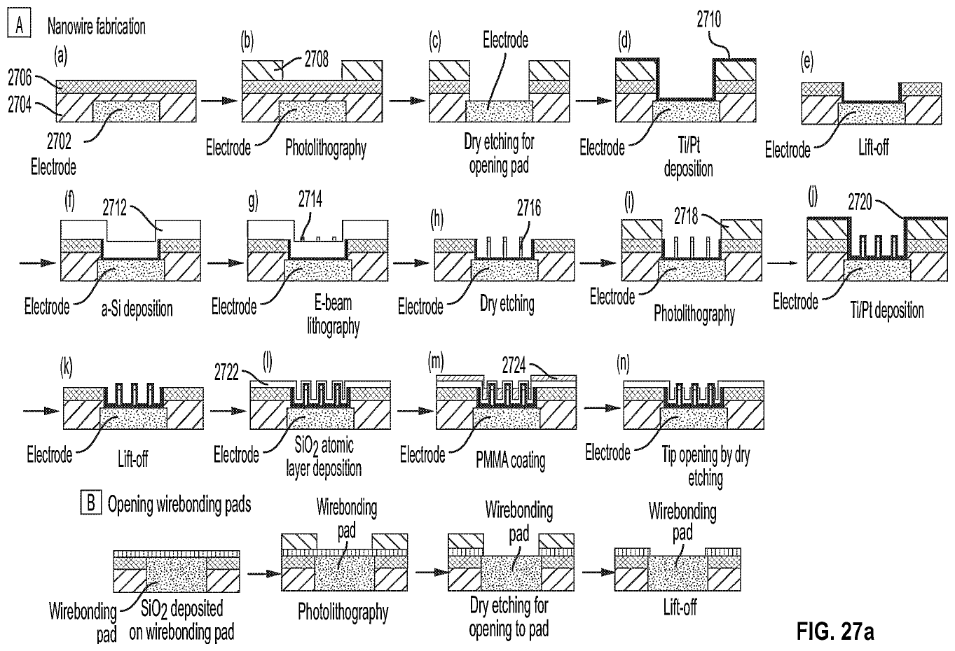

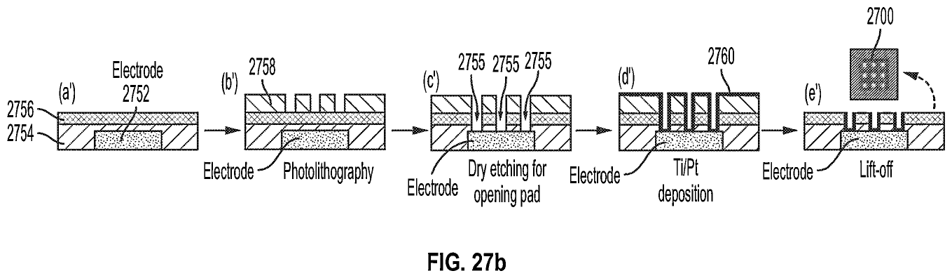

FIG. 27a schematically illustrates cross-sectional views of an example of a fabrication process for a nanowire-based electrode device, including a process A for nanowire fabrication, for producing an electrode comprised of an array of nanowires, and a process B for fabricating an opening to a wire-bonding pad. FIG. 27b schematically illustrates cross-sectional views of an example of a fabrication process for an electrode comprised of a hole array, according to some embodiments of the technology disclosed herein. A dashed arrow at the right-hand side of FIG. 27b points to an inset showing a plan view of an optical image of a hole array 2700 produced by the illustrated process. From a quick comparison of FIGS. 27a and 27b, it is evident that an electrode comprised of a hole array, according to some embodiments of the present technology, may be formed by a much simpler process than that for forming an electrode comprised of an array of nanowires. More specifically, in order to produce an array of sharp nanowires, at least fourteen (14) steps may be required (see process A of FIG. 27a); in contrast, in order to produce a hole array, as few as four (4) steps may be sufficient (see FIG. 27b). In some embodiments, these four steps may be adapted from initial steps (a) through (e) of process A (see upper row of FIG. 27a). According to some embodiments of the present disclosure, an electrode comprised of a hole array that includes a plurality of holes (e.g., two or greater than two, or greater than five, or at least nine, etc.) may result in the electrode having a lower impedance than a configuration in which an electrode is comprised of a same plurality of nanowires. Although not wishing to be bound by theory, the lower impedance may arise from a relatively a larger surface area for contacting an object under investigation (e.g., a biological cell) than the surface area associated with an array of nanowires. Lower impedance (or higher conductance) may be of critical importance for recordings for electrophysiological investigations, as it not only may facilitate a higher degree of intracellular coupling, which may increase a signal-to-noise ratio, but also may reduce signal attenuation.

As described herein, the initial steps of process A of FIG. 27a may result in a generally planar recessed electrode overlayed with a well-shaped layer of PtB. Lithography for forming nanowires may not begin until after step (e) of process A. In contrast, in some embodiments of the present disclosure, a same number of steps may be sufficient to form an electrode comprised of a hole array, as depicted by steps (a′) through (e′) of FIG. 27b. The inset at step (e′) of FIG. 27b shows an embodiment of the disclosed technology in which the hole array 2700 is a 3×3 array of nine (9) holes comprised of generally circular openings or recesses when viewed from above (e.g., in a plan view). It should be understood that the present technology is not limited to hole arrays having nine circular holes. In some embodiments of the present technology, a hole array may have other than nine holes and/or may have at least hole that does not have a circular shape. Any suitable shape may be used for the holes of the hole array.

Returning to FIG. 27a, process A for fabricating an array of nanowires may proceed as follows: At (a), a starting structure is provided in which a pad portion of a conductive electrode 2702 is surrounded by and covered with an insulative layer 2704, which may be a layer of silicon oxide (e.g., SiO₂). The insulative layer 2704 may be covered by an insulative layer 2706, which may be a layer of silicon nitride (e.g., Si₃N₄). Although not shown in FIG. 27a, the starting structure may sit on top of circuitry for addressing the electrode 2702 (e.g., stimulation circuitry, recording or sensing circuitry, etc.). At (b), photolithography may be used to form an opening in the insulative layers 2704, 2706 using a patterned layer of photoresist 2708. At (c), the insulative layers 2704, 2706 may be etched through the patterned photoresist 2708 to expose a surface of the pad portion of the electrode 2702. The etching may involve one or more known dry etching technique(s), which may result in vertical sidewalls adjacent the exposed surface of the electrode 2702. At (d), before the patterned layer of photoresist 2708 is removed, the sidewalls and the exposed surface of the electrode 2702 may be coated with a conductive layer 2710. The conductive layer 2710 may be comprised of titanium and platinum (e.g., a Ti/Pt multilayer structure) between a layer of PtB and the surface of the electrode 2702 and the sidewalls. At (e), the pattern layer of photoresist 2708 may be removed in a lift-off process, resulting in the electrode 2702 having a recessed structure comprised of a well-shaped conductive layer 2710 disposed above the pad portion of the electrode 2702. At (f), an insulative layer 2712 may be deposited over the conductive layer 2710. The insulative layer 2712 may comprise amorphous silicon. At (g), electron-beam lithography may be used to produce a patterned hard-mask 2714 on the insulative layer 2712. The hard-mask 2714 may be comprised of a patterned layer of a solid material that is non-reactive with chemicals used in photolithography. For example, the hard-mask 2714 may be comprised of a non-reactive metal. At (h), the insulative layer 2712 may be etched through the hard-mask 2714 to obtain narrow insulative pillars 2716 that may extend vertically above the conductive layer 2710 overlaying the surface of the pad portion of the electrode 2702. The etching may involve one or more known dry etching technique(s), which may result in the pillars 2716 having vertical profiles extending generally perpendicularly above the electrode 2702. At (i), photolithography may be used to mask the insulative layer 2706 with a patterned layer of photoresist 2718 while leaving surfaces of the pillars 2716, the hard-mask 2714, and the conductive layer 2710 exposed. At (j), before the patterned layer of photoresist 2718 is removed, the exposed surfaces may be coated with a conductive layer 2720, which may be comprised of Ti/Pt and PtB, similar to the conductive layer 2710. At (k), the patterned layer of photoresist 2718 may be removed via a lift-off process, which may result in the pillars 2716 extending vertically to a height that is above a height of the insulative layer 2706. At (1), exposed surfaces may be coated with an insulative layer 2722. For example, the insulative layer 2722 may be a layer of silicon oxide deposited by atomic layer deposition. At (m), photolithography may be used to form openings that expose upper tips of the pillars 2716 using a patterned layer of photoresist 2724. For example, the patterned photoresist 2724 may be formed of a polymethyl methacrylate or PMMA material, which may permit finer features to be patterned via exposure to light having wavelengths shorter than wavelengths of visible light. At (n), the insulative layer 2722 may be removed from around the tips of the pillars 2716 (e.g., by dry etching) such that the conductive coating 2720 on the tips of the pillars 2716 is exposed. The array of nanowires may correspond to the pillars 2716 extending vertically above the surface of the pad portion of the electrode 2702. The conductive coating 2720 on the tips of the pillars 2716 may be contact surfaces and may serve as contact portions of the electrode 2702. The contact surfaces may physically interface or contact an object being investigated during a electrophysiological investigation.

Referring now to FIG. 27b, a process for fabricating a hole array, according to some embodiments of the present technology, may proceed as follows: At (a′), a starting structure is provided in which a pad portion of a conductive electrode 2752 is surrounded by and covered with an insulative layer 2754, which may be a layer of silicon oxide (e.g., SiO₂). The insulative layer 2754 may be covered by an insulative layer 2756, which may be a layer of silicon nitride (e.g., Si₃N₄). The insulative layers 2754, 2756 may be referred to collectively as a “passivation layer” herein. Although not shown in FIG. 27b, the starting structure may sit on top of circuitry for addressing the electrode 2752 (e.g., a pixel circuit comprised of stimulation circuitry, recording or sensing circuitry, etc.). At (b′), photolithography may be used to form openings in the insulative layers 2754, 2756 using a patterned layer of photoresist 2758. At (c′), the insulative layers 2754, 2756 may be etched through the patterned photoresist 2758 to form a plurality of openings 2755 that expose a surface of the pad portion of the electrode 2752. The etching may involve one or more known dry etching technique(s), which may result in the openings 2755 being recesses having vertical sidewalls extending perpendicularly from the surface of the pad portion of the electrode 2752. At (d′), before the patterned layer of photoresist 2758 is removed, the sidewalls and the exposed surface of the electrode 2752 may be coated with a conductive layer 2760. The conductive layer 2760 may be comprised of titanium and platinum (e.g., a Ti/Pt multilayer structure) on top of the electrode 2752 and a player of PtB on top of the titanium and platinum. At (e′), the pattern layer of photoresist 2758 may be removed in a lift-off process, resulting in a plurality of openings 2755 having surfaces coated or lined with the conductive layer 2760. The openings 2755 may be in physical and electrical contact with the pad portion of the electrode 2752 and may correspond to holes of the hole array. The conductive coating 2760 on the vertical sidewalls and bottoms of the openings 2755 may be contact surfaces and may serve as contact portions of the electrode 2752. The contact surfaces may physically interface or contact an object being investigated during a electrophysiological investigation.

As schematically depicted in FIGS. 27a and 27b, a surface area of the contact surfaces of the openings 2755 may be relatively larger in comparison with a surface area of the contact surfaces of the pillars 2716, which may give rise to a higher intracellular coupling efficiency and consequently a lower impedance of signals recorded during the investigation when using electrodes comprised of hole arrays relative to electrodes comprised of arrays of nanowires.

According to some embodiments of the technology disclosed herein, the bottom of each of the openings 2755 may have a dimension (as measured in a direction parallel to the surface of the pad portion of the electrode 2752) of between 0.5 μm and 5 μm, or between 1 μm and 3 μm, or between 1.5 μm and 2.5 μm. In some embodiments, the openings 2755 may have a circular shape when observed in a plan or “bird's eye” view, and the dimension may be a diameter of the openings 2755. In some embodiments, the openings 2755 may have a square shape when observed in a plan view, and the dimension may be a width of a side of the openings 2755. It should be understood that these are non-limiting examples of the types of shapes of the openings 2755. In some embodiments, the openings 2755 may have shapes other than circles or squares and may even have an asymmetrical shape.

According to some embodiments of the technology disclosed herein, a depth of each of the openings 2755, extending along a direction perpendicular to the surface of the pad portion of the electrode 2752, may be is between 1 nm and 5 μm, or between 1 nm and 2 μm, or between 0.5 μm and 2 μm. The depth may correspond to a height of the sidewalls of the openings 2755.

According to some embodiments of the technology disclosed herein, for openings 2755 shaped as squares (or circles), a depth to width (or diameter) aspect ratio may be 1:1 or between 1:1000 and 1000:1, or between 1:100 and 100:1, or between 1:10 and 10:1, or 1:1, or between 1:10 and 1:1.

As noted above, the conductive layer 2760 coating the openings 2755 (i.e., the holes of the hole array) may be comprised of PtB. In some embodiments of the present technology, the PtB may be deposited using cyclic voltammetry and may have a nano-scale roughness, Ra, that can increase conductance. In some embodiments, the conductive sidewalls of a hole may have a surface roughness, Ra, of between 0.1 nm and 100 nm, or between 0.1 nm and 10 nm, or between 1 nm and 10 nm. Furthermore, the contactable surface area of the hole, which may come into contact to an object under investigation (e.g., a cell), may be selectively increased by increasing one or more dimension(s) of the hole (e.g., diameter (width) and/or depth). As noted above, a larger surface area may result in an improved impedance during electrophysiology studies. In one non-limiting example, the hole may have a diameter of 2 μm, a depth of 500 nm, and a center-to-center distance to an adjacent hole of 4 μm. In some embodiments, the dimensions of the holes of a hole array may be controlled by controlling fabrication parameters affecting the holes' depth and/or diameter/width. For example, the holes may have a diameter that is controlled to be in a range of 1 μm to 4 μm. Similarly, the holes may have a depth that is controllable based at least in part on a thickness of the insulative layer 2756 and a thickness of the insulative layer 2754 above the electrode 2752. In some embodiments, the holes may have a depth in a range of 2 μm to 2 nm. In some embodiments, the PtB forming the conductive layer 2760 may be deposited by cyclic voltammetry after packaging (e.g., as a post-production step).

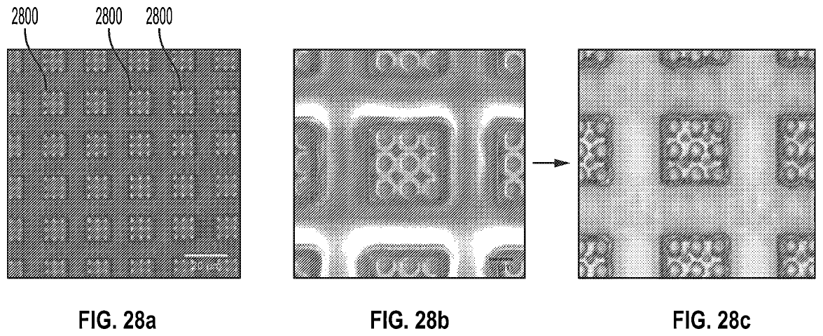

According to some embodiments of the present disclosure, an electrode may be comprised of more than one hole array, each of which may be referred to herein as a sub-array. In some embodiments, holes of a sub-array may be arranged in M columns×N rows, where M and N are integers greater than 1. FIG. 28a shows a micrograph of an example of a portion of an electrode comprised of an array of sub-arrays 2800. Each sub-array 2800 may be comprised of nine (9) holes in a 3×3 arrangement. The sub-arrays 2800 of FIG. 28a may be similar to the hole array 2700 in the inset of (e′) in FIG. 27b. In FIG. 28a, each sub-array 2800 or 3×3 arrangement of holes may correspond to a single electrode pad (e.g., the “electrode” portion FIG. 27b). That is, an overall contactable surface area of each electrode pad may be comprised of a sum of the contactable surface areas of the nine (9) holes disposed above a main surface of the electrode pad, which may result in an increased intracellular coupling efficiency, as noted above. In some embodiments, the holes in a sub-array may have a center to center distance of between 1 μm and 10 μm, or between 2 μm and 10 μm, or between 3 μm and 5 μm. In some embodiments, a lateral distance between a portion of a hole in a sub-array and an edge of an electrode pad on which the sub-array sits may be less than 5 μm, or less than 3 μm, or less than 2 μm. In some embodiments, an electrode pad may have a lateral dimension, measured in a direction parallel to the main surface of the electrode pad, between 1 and 50 μm, or between 1 and 20 μm, or between 10 and 20 μm. FIG. 28b shows a micrograph of a sub-array structure prior to deposition of a PtB conductive layer (e.g., at (c′) in FIG. 27b prior to deposition of the conductive layer 2760). FIG. 28c shows a micrograph of a sub-array structure after deposition of PtB in the holes (e.g., at (d′) in FIG. 27b after deposition of the conductive layer 2760).

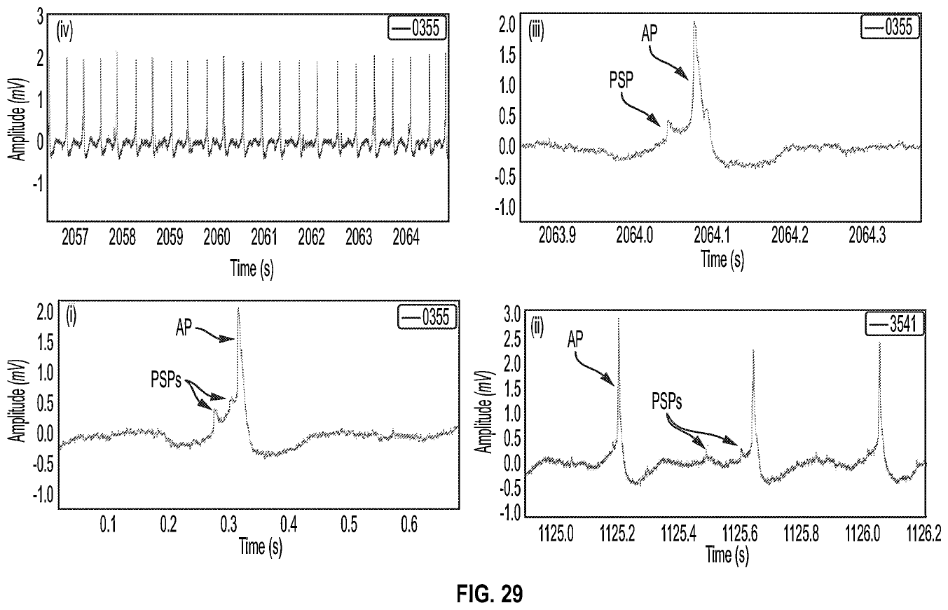

FIG. 29 shows charts (i) through (iv), which are examples of experimental recording results obtained using electrodes having a sub-array of 3×3 holes. Each of the charts (i) through (iv) shows signal amplitude (in mV or millivolts) as a function of time (in s or seconds). Chart (i) shows signal amplitude recorded during a time period of about 0.1 s to about 0.6 s of an investigation. Chart (ii) shows signal amplitude recorded during a time period of about 1125.0 s to about 1126.2 s of the investigation. Chart (iii) shows signal amplitude recorded during a time period of about 2063.9 s to about 2064.3 s of the investigation. Chart (iv) shows signal amplitude recorded during a time period of about 2057 s to about 2064 s of the investigation. The charts (i) through (iv) demonstrate a high degree of intracellular coupling, with easily identifiable potentials (APs) and with a sub-threshold signal resolution that permits postsynaptic potentials (PSPs) to be readily discerned.

According to some embodiments of the technology disclosed herein, a device for performing an intracellular electrophysiological investigation may comprise an array of electrodes. In some embodiments, the array may include hundreds of electrodes (e.g., at least 500 electrodes, or at least 750 electrodes) In some embodiments, the array may include at least 1000 electrodes (e.g., at least 1500 electrodes, or at least 2000 electrodes, or at least 2500 electrodes, or at least 3000 electrodes). In some embodiments, each electrode may include a hole array (e.g., 3×3 array of holes) or a plurality of sub-arrays (e.g., two or more multi-hole arrays). Using CMOS fabrication technologies, the electrodes may be designed to have desired electrode-pad dimensions and a desired pad-to-pad spacing, as well as desired hole dimensions and a desired hole-to-hole spacing appropriate for the object being investigated.

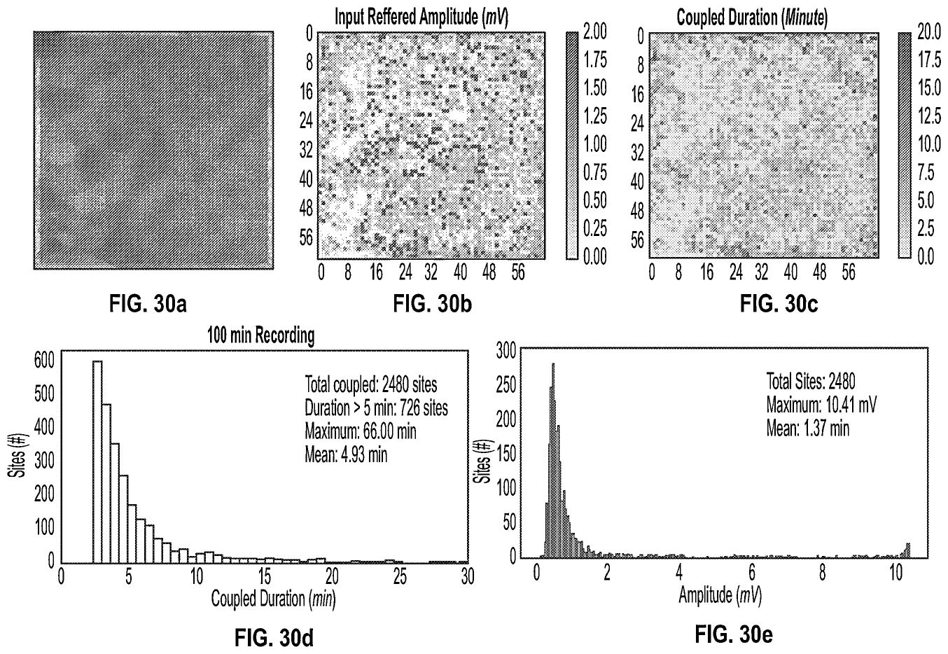

FIG. 30a shows an image of an array of electrodes produced according to an embodiment of the technology disclosed herein, and FIGS. 30b through 30e illustrate intracellular coupling capabilities of the array of electrodes using a DC stimulation current injected to the electrodes. Each of the electrodes includes a hole array comprised of a plurality of holes. The array includes 4096 electrode sites in a 64×64 arrangement, as shown in the plan view of FIG. 30a. FIG. 30b shows signal amplitudes recorded for the electrode sites during an investigation. In FIG. 30b, darkness levels are coded according to the bar on the right to indicate voltages in mV. FIG. 30c shows coupling durations recorded for the electrode sites during the investigation. A coupling duration for an electrode site may be determined by an amount time during which a signal may be recorded from the electrode site. In FIG. 30c, darkness levels are coded according to the bar on the right to indicate coupling times in minutes. As evident from FIGS. 30b and 30c, although intracellular coupling was not observed for all (i.e., 100%) of the electrodes sites, data from the investigation indicates that intracellular coupling was observed in nearly 70% of the electrode sites and, in some cases, up to 97% of the electrode sites. FIG. 30d shows a distribution chart for coupling duration, indicating how many of the electrode sites were able to provide a recorded signal as a function of time. FIG. 30e shows a distribution chart for signal amplitude, indicating how many of the electrode sites were able to provide a recorded signal as a function of signal amplitude. The results of the investigation indicate that intracellular coupling using electrodes comprised of hole arrays may occur for over two hours for some electrode sites, which is believed to be an improvement over electrodes based on nanowires (see [5]).

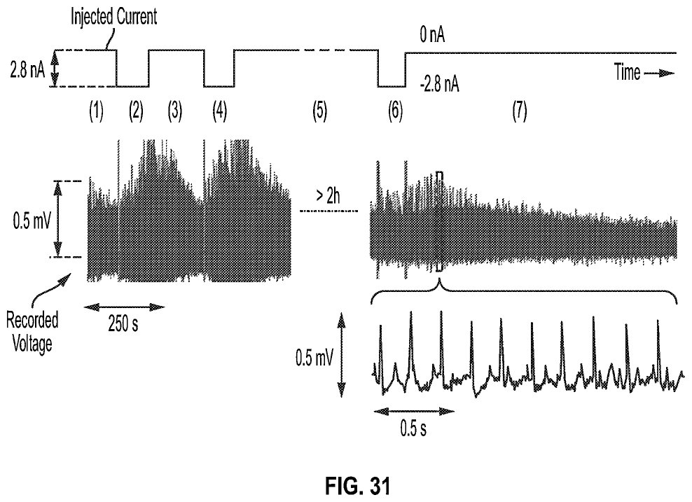

According to some embodiments of the technology disclosed herein, in order to extend or prolong the coupling duration of an electrode, the electrode may be injected with a feedback-based stimulation current during recording. More specifically, in some embodiments, instead of injecting the electrode with a DC current that is held constant during recording, the current injected to the electrode may be toggled on and off during recording. The toggling may occur based on a characteristic of a signal recorded for the electrode. FIG. 31 shows an example of feedback-based stimulation, an embodiment of the disclosed technology. Initially, during region (1) of the time sequence for the injected current, the electrode may not be injected with a current, i.e., 0 nA of current may flow to the electrode. A signal recorded for the electrode during region (1) may be a signal corresponding to background noise of approximately 0.03 mV peak to peak, as pre-characterized for the system used in the investigation. During region (2) of the time sequence, a current of −2.8 nA may flow to the electrode corresponding to 2.8 nA flowing in a negative direction. While the electrode is stimulated with −2.8 nA, intracellular coupling may be established, as indicated by the increasing amplitude of the recorded voltage during region (2). Region (2) may end after a predetermined period of time or after a predetermined amplitude is reached for the recorded voltage. In some embodiments, stimulation may occur for a predetermined time period in a range of about 100 s to 150 s (e.g., 120 s). Regions (1) and (2) may take place over a total time period in a range of about 200 s to 300 s (e.g., 250 s). During region (3) current injection may stop and may remain at 0 nA until the amplitude of the recorded voltage decays to a predetermined level (e.g., the level of background noise, or within 5% of a prior peak signal, or within 2% of the prior peak signal). When the predetermined level is reached, the electrode may be injected with −2.8 nA of current again, commencing region (4). Similar to region (2), region (4) may end after a predetermined period of time or after a predetermined amplitude is reached for the recorded voltage. During region (5), similar to region (3), current injection may stop and may remain at 0 nA until the amplitude of the recorded voltage decays to a predetermined level. The decay in the recorded voltage to the predetermined level may occur over a time period of hours (e.g., greater than 2 hours). Region (6) may be a repeat of region (2), and region (7) may be a repeat of region (5). An inset at the lower right portion of FIG. 31 shows an expanded portion of the recorded voltage during the time period delimited by the vertical broken lines in region (7). The expanded portion reveals that with feedback-based stimulation it is possible to record details of APs and PSPs (see FIG. 29 at (ii)) even when no current is flowing to the electrode, and even after over two hours of recording has already occurred.

The hole arrays and the hole sub-arrays described above may be generally planar from hole to hole (e.g., the hole 2700) or sub-array to sub-array (e.g., the sub-array 2800), except for typical surface contours resulting from various fabrication techniques (e.g., accumulation of deposition material at corners, etc.). The inventors have recognized and appreciated that improved measurements may be achieved by providing an elevated wall surrounding each hole of a hole array, or around each sub-array of holes, as discussed below.

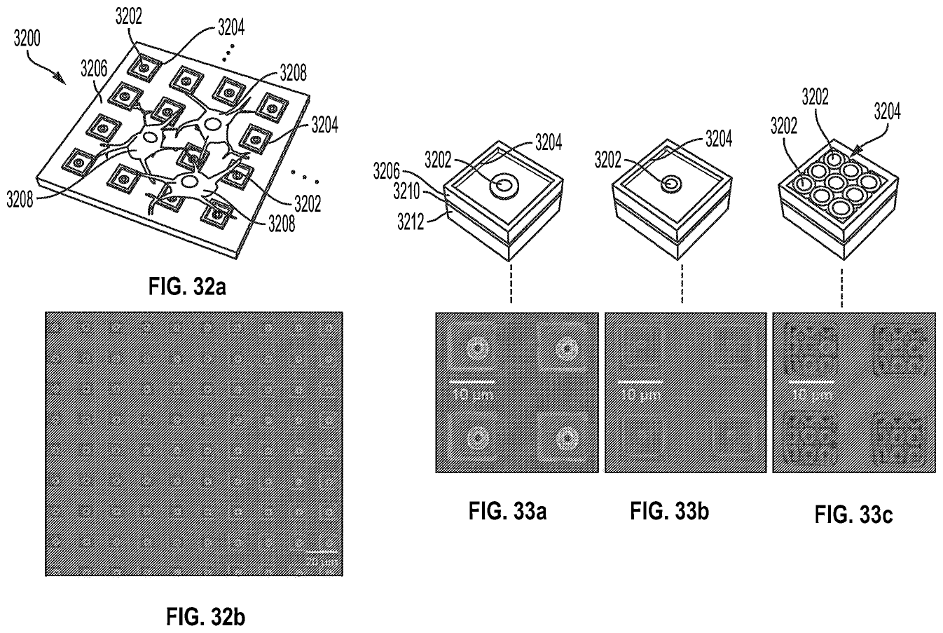

FIG. 32a schematically depicts a hole array 3200, according to some embodiments of the present technology. The hole array 3200 may be a 4×4 array of individual holes 3202, as shown, or may be part of a larger array of holes (e.g., a 64×64 array of individual holes). FIG. 32b shows a micrograph of a portion of a hole array of individual holes fabricated according to some embodiments of the present technology, discussed below. Each hole 3202 may be recessed below a main surface of an insulative passivation layer 3206, and each hole 3202 may be surrounded by an insulative wall 3204 extending above the main surface of the passivation layer 3206. Although the passivation layer 3206 is schematically depicted to be a single layer, the passivation layer 3206 may be comprised of a plurality of insulative layers. In some embodiments, the passivation layer 3206 may be comprised of a lower layer of silicon oxide and an upper layer of silicon nitride overlaying the lower layer of silicon oxide. The wall 3204 may be shaped as a square, as shown, or may be shaped as a circle, or a triangle, or any shape that surrounds the hole 3202. In some embodiments, the wall 3204 may be sized and shaped to permit the wall 3204 to be covered by a cell. For example, in FIG. 32a, the wall 3204 is sized such that a single cell 3208 may cover an individual hole 3202 and the wall 3204 surrounding the hole 3202. The cell 3208 depicted in FIG. 32a is a neuron cell but it should be understood that the hole array 3200 may be used with other types of cells and/or non-cell organisms.

According to some embodiments of the present technology, the portion of the hole array 3200 comprised of wall 3204 may resemble a well in the hole array 3200 and therefore the wall 3204 may also be referred to herein as the well 3204. In some embodiments, the well 3204 may have a depth that is limited by a thickness of the passivation layer 3206. In some embodiments, the thickness of the passivation layer 3206 may be greater than 2 μm (e.g., 2.5 μm or 3.0 μm or more), and the depth of the well 3204 may be in a range of 0 μm to about 2 μm (e.g., 0.5 μm, or 1 μm, or 1.5 μm or 2 μm). When the depth is 0 μm, the hole array 3200 may resemble the hole array 2700 discussed above (see FIG. 27b). In some embodiments, the well 3204 may be shaped as a square and may have a dimension of 10 μm on each side of the square. In some embodiments, the well may be shaped as a circle having a diameter of 10 μm. With a side or a diameter of 10 μm, the well 3204 may be suitable for use with cells that typically having dimensions that are larger than 10 μm.

According to some embodiments the hole 3202 in the well 3204 (i.e., the hole surrounded by the wall 3204) may be circular and may have a diameter that is limited by the dimension of the well 3204. In some embodiments, the hole 3202 may have a diameter in a range of about 1.5 μm to less than 10 μm. In some embodiments, a depth of the hole 3202 may be in a range of about 0.5 μm to about 2 μm (e.g., 0.5 μm or 1 μm or 1.5 μm or 2 μm).

According to some embodiments of the present technology, the wall 3204 may surround a single hole 3202 or may surround a sub-array of a plurality of holes 3202. FIGS. 33a and 33b each schematically shows a well 3204 surrounding a single hole 3202 and a micrograph of an arrangement of four 10 μm×10 μm wells and their corresponding holes. In some embodiments, adjacent 10 μm×10 μm wells 3204 may be separated by a distance of about 10 μm. The hole in FIG. 33a is relatively larger in diameter than the hole in FIG. 33b. The larger hole 3202 of FIG. 33a may provide a larger surface area for contacting the cell 3208 than the smaller hole 3202 of FIG. 33b. As discussed above, a larger hole surface area that may contact the cell 3208 may be associated with a lower electrical impedance or a higher conductance, which may lead to more robust signals recorded for cells. As discussed herein, each hole 3202 may be considered an electrode or a portion of an electrode and may be a vertical extension of an electrode pad on which the hole 3202 is fabricated. However, as hole size increases there is also an increased probability that the hole 3202 does not get fully covered by a single cell 3208. That is, the probability of the larger hole 3202 (FIG. 33a) being fully covered by a single cell 3208 is lower than a probability of the smaller hole 3202 (FIG. 33b) being fully covered, and therefore there is a higher probability that a current injected in to the cell 3208 may have some leakage into a solution surrounding the cell 3208 and the hole 3202. As such, the larger hole 3202 (FIG. 33a) is not necessarily better than the smaller hole 3202 (FIG. 33b). In some embodiments, the hole array 3200 may be comprised of a plurality of holes 3202 of various dimensions, some larger, some smaller, and some in between, and the holes 3202 may be surrounded by walls 3204 of various dimensions, some larger, some smaller, some in between. Such variations in dimensions for the holes 3202 and/or for the walls 3204 may permit a single investigation to yield recordings under a plurality of different cell-hole conditions, which may permit a determination of an optimal hole structure (e.g., hole diameter, hole depth) and/or an optimal wall structure (e.g., wall (well) depth, well diameter) for a particular type of cell. FIG. 33c shows a well 3204 surrounding a sub-array comprised of nine holes 3202 and a micrograph of an arrangement of four 10 μm×10 μm wells each surrounding nine holes. The nine holes 3202 are all connected to a same electrode pad and may be associated with a same electrode. An increase in the number of holes 3202 surrounded by the well may increase surface area for contacting the cell 3028, which may lead to a higher conductance than a single hole 3202. However, fabrication of sub-arrays may be more complex and costly. In some embodiments, the hole array 3200 may be comprised of a plurality of holes 3202 of various dimensions and/or a plurality of sub-arrays each comprised of a plurality of holes 3202. In some embodiments, some of the sub-arrays may be comprised of fewer holes while some of the sub-arrays may be comprised of smaller holes. As will be appreciated, the hole array 3200 may have other combinations of individual holes and/or sub-arrays of holes not specifically described herein.

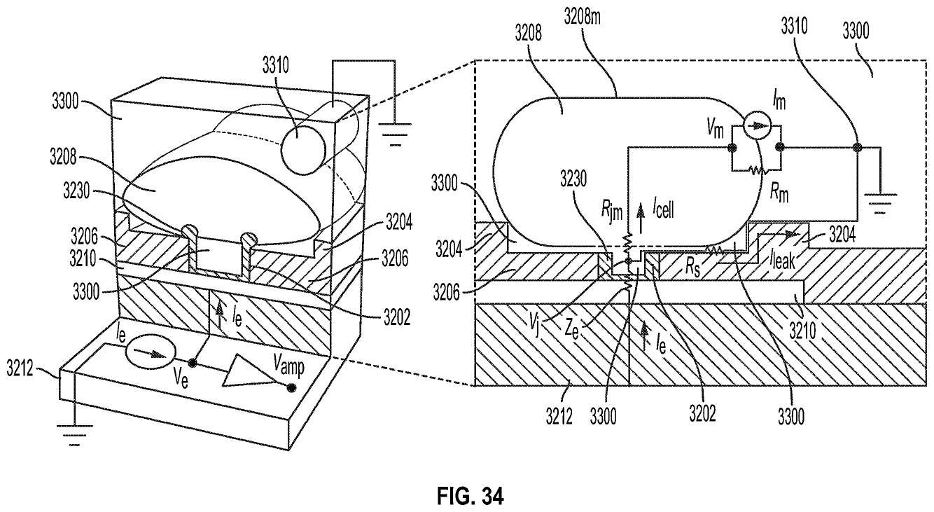

Beneficial aspects of the wall 3204 may be understood from FIG. 34, which schematically shows an interface between the cell 3208 and circuitry underneath the cell 3208. Included in FIG. 34 is an inset showing a circuit model for the interface. According to some embodiments of the present technology, the cell 3208 may be in a solution 3300 and may be situated on top of the hole 3202, which also is in the solution 3300. That is, the hole array 3200, of which the hole 3202 is a part, may be in the solution 3300, and FIG. 34 shows only a portion of the hole array 3200. Included in the solution 3300 may be a counter electrode 3310, which may be formed of any suitable non-reactive material (e.g., Pt).

According to some embodiments of the present technology, the hole 3202 may be comprised of a PtB layer 3230 covering surfaces of the hole 3202. The PtB layer 3230 may be deposited by electrodeposition techniques, which may cause a bulbous portion to be present at corners (e.g., an outer rim of the hole 3202), as schematically depicted in FIG. 34. The PtB layer 3230 may be electrically in contact with an electrode pad 3210, such that the PtB layer 3230 may be a vertical extension of the electrode pad 3210. The electrode pad 3210, which may be a layer of metal (e.g., aluminum), may be disposed above a circuitry layer 3212. In some embodiments, the circuitry layer 3212 may be comprised of one or more integrated circuits fabricated on a same chip as the hole array 3200. For example, the circuitry layer 3212 may be comprised of stimulation circuits configured to output a stimulation current and/or a stimulation voltage to the hole 3202 via the electrode pad; one or more recording or sensing circuits configured to sense a signal (e.g., a voltage) at the hole 3202; and other on-chip circuitry suitable for use in investigations involving the hole array 3200. As noted above, the hole 3202 may be recessed below a main surface of the passivation layer 3206 and may be surrounded by the wall 3204 extending above the main surface of the passivation layer 3206. In some embodiments, the wall 3204 may be formed by lithographically patterning the passivation layer 3206 and removing material such that the wall 3204 remains, as described below. In some embodiments, the walls 3204 of the hole array 3200 may be sized to be completely or nearly completely covered by the cell 3208. That is, an area bounded by the wall 3204 (“wall area”) may be comparable to an area of the cell 3208 so as to be covered by the cell 3208. A benefit to having the wall area fit within the area of the cell 3208 may be understood from the circuit model in the inset of FIG. 34.

In some investigations it may be desirable to measure a membrane potential, Vₘ, at a membrane 3208a of the cell 3208. According to some embodiments of the present technology, the membrane potential, Vₘ, may be modeled according to the circuit model in the inset of FIG. 34. In FIG. 34, Rₘ is an impedance of the membrane 3208m and Iₘ is a current through the membrane 3208m. The inventors have recognized and appreciated that it may be difficult to measure the membrane potential, Vₘ; however, the inventors also have recognized that a reasonable estimate of the membrane potential, Vₘ, may be determined by measuring a junction voltage, Vⱼ, at a cell-electrode junction, provided that impedances and other losses are minimized. In the circuit model, minimization of a junction-membrane impedance, Rⱼₘ, and/or minimization of an electrode impedance, Zₑ, may result in the junction voltage, Vⱼ, having a value that is closer to the membrane potential, Vₘ, than in the absence of such minimization.

According to some embodiments of the present technology, during a measurement process, stimulation circuitry in the circuitry layer 3212 may output a known electrode current Iₑ, which may be injected from the PtB layer 3230 to the cell 3208, which may promote membrane permeabilization or electroporation of the membrane 3208m. In order to promote minimization of the junction-membrane impedance Rim and/or minimization of the electrode impedance, Zₑ, the electrode current, Le, may be increased to increase the amount of electroporation of the membrane 3208m. Ideally, the electrode current, Iₑ, may equal the membrane current, Iₘ. The inventors have recognized and appreciated that a cell current, I_{cell}, injected into the cell 3208 may not equal the electrode current, Iₑ, due to a leakage current, Iₗₑₐₖ, leaking into the solution 3300. For example, the leakage current, Iₗₑₐₖ, may arise from suboptimal contact between the cell 3208 and the PtB layer 3230, such that the cell 3208 does not fully cover the hole 3202. The inventors also have recognized that the leakage current, Iₗₑₐₖ, may be minimized by blocking possible paths for movement of current from the PtB layer 3230 into a bulk portion of the solution 3300. The inventors have recognized that easy fluid flow from a region near a portion of the PtB layer 3230 not covered by the cell 3208 into the bulk of the solution 3300 may be an escape path for current to dissipate easily into the solution 3300 and contribute to the leakage current, Iₗₑₐₖ, and the inventors have further recognized that by minimizing a size of this escape path the leakage current, Iₗₑₐₖ, may be reduced or possibly even eliminated. In some embodiments, the escape path may be minimized by the wall 3204, which may function to restrict or even prevent movement of the solution 3300 from under the cell 3208. That is, when the cell 3208 sits on the wall 3204 and covers all or nearly all of the wall 3204, the solution 3300 solution located with the wall 3204 (i.e., under the cell 3208) is trapped and cannot flow freely in the bulk portion of the solution 3300 outside of the wall 324. In some embodiments, even if a small part of the wall 3204 is not covered by the cell 3208, there may be sufficient restriction of flow of the solution 3300 to reduce a magnitude of the leakage current, Iₗₑₐₖ, relative to a magnitude of the leakage current, Iₗₑₐₖ, when the wall 3204 is not present. Thus, by minimizing the leakage current, Iₗₑₐₖ, the cell current, I_{cell}, may be approximated by the electrode current, Iₑ. In some embodiments, the cell current, I_{cell}, may be used to approximate the membrane current, Iₘ, by assuming a fixed cell impedance as current that is injected into the cell 3208 travels through the cell 3208. In the circuit model, the presence of the wall 3204 may be represented by a solution-sealing impedance, Rₛ, that restricts movement of the solution 3300 and thus reduces the leakage current, Iₗₑₐₖ.

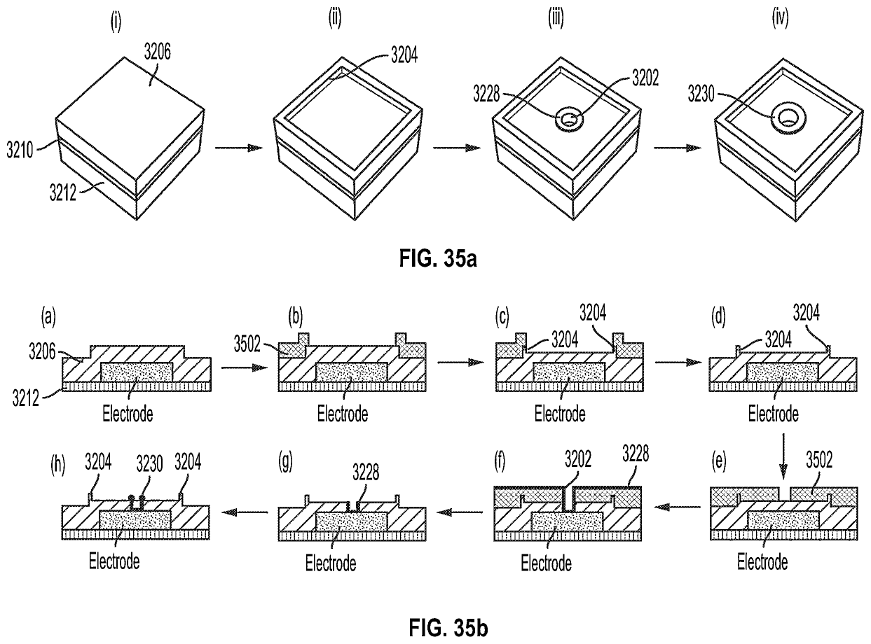

FIG. 35a schematically illustrates a general process flow for forming a hole 3202 of a hole array 3200, according to some embodiments of the present technology. At (i), in some embodiments, a starting structure for the hole array 3200 may be provided on a chip or semiconductor substrate. Only a portion of the starting structure is depicted in FIG. 35a. The starting structure may be comprised of a circuitry layer 3212, an electrode pad 3210 overlaying the circuitry layer 3212, and a passivation layer 3206 overlaying the electrode pad 3210. The chip or substrate may be processed using known CMOS fabrication techniques and/or other thin-film processing techniques to produce the circuitry layer 3202. In some embodiments, the circuitry layer 3202 may be comprised of electrode circuitry relevant to the hole 3202 to be formed (e.g., simulation circuitry, sensing and recording circuitry, etc.). The electrode pad 3210 may be formed directly above the circuitry layer 3212 and may be operably connected to the electrode circuitry of the circuitry layer 3212. One or more wiring interconnection layers (not shown) may be disposed between the circuitry layer 3212 and the electrode pad 3210. In some embodiments, the electrode pad 3210 may be positioned directly above the electrode circuitry of the circuitry layer 3212 to minimize an amount of wiring for interconnecting the electrode pad 3210 and the electrode circuitry. At (ii), in some embodiments, a well 3204 is formed in the passivation layer 3206 such that the well 3204 is bordered by a raised peripheral wall surrounding a sunken region. As noted above, although the well 3204 may be depicted to have a square shape in FIG. 35a, in some embodiments the well 3204 may have another shape. For example, the well 3204 may alternatively have a circular shape and may comprise a raised circular border wall surrounding a circular sunken region. At (iii), in some embodiments, a portion of the sunken region of the well 3204 may be etched into the electrode pad 3210 to form the hole 3202. Standard photolithography and etching techniques may be used to form the well 3204 and/or the hole 3202 to have a desired dimension and/or a desired depth and/or a desired shape. In some embodiments, directional etching techniques (e.g., reactive-ion etching) may be used, in order to provide the well 3204 and/or the hole 3202 with a vertical border wall with minimal sloping. In some embodiments, bottom and side surfaces of the hole 3202 may be coated with a Ti/Pt layer 3228, such that the surfaces of the hole 3202 serve as conductive extensions of the electrode pad 3210. For example, the hole 3202 as etched may be coated with a layer of titanium (Ti) followed by a layer of platinum (Pt), using known thin-film deposition techniques (e.g., sputtering), to form the Ti/Pt layer 3228. At (iv), a layer of PtB 3230 may be formed on the Ti/Pt layer 3228 coating the surfaces of the hole 3202. As will be appreciated, the hole 3230 may remain a hole or recessed structure after the layer of PtB 3230 is formed, and may not be completely filled by the layer of PtB 3230.

FIG. 35b schematically illustrates cross-sectional views of an example of a fabrication process for the hole 3202, according to some embodiments of the present technology. In some embodiments, the starting structure described above may be provided at (a). At (b), the starting structure may be patterned using known photolithographic techniques to define the well 3204. For example, photoresist 3502 may be patterned on the passivation layer 3206 to forming an opening shaped as a square or a circle for etching the passivation layer 3206. At (c), the passivation layer 3206 is etched through the opening in the photoresist 3502 to form the sunken region of the well 3204. For example, a direction etching technique may be used, such as reactive-ion etching, which may result in vertical sidewall of the well 3204 remaining unetched under the photoresist 3502. In some embodiments, the passivation layer 3206 may be comprised of a layer of silicon oxide under a layer of silicon nitride, with the layer of silicon oxide surrounding the electrode pad 3210 and with the layer of silicon nitride above the electrode pad 3210 and above the silicon oxide surrounding the electrode pad 3210, such that the silicon nitride of the passivation layer 3206 is etched to form the well 3204. At (d), the photoresist 3502 may be removed, such that the well 3204 remains. At (e), photoresist 3502 may be patterned on the well 3204 to form an opening for defining the hole 3202. At (f), the passivation layer 3206 exposed through the opening in the photoresist 3502 at (e) may be etched through to the electrode pad 3210 to form the hole 3202 and, subsequently, before removal of the photoresist 3502, a Ti/Pt layer 3228 is deposited to coat the bottom and sidewall surfaces of the hole 3202 as well as the photoresists 3502. The etching of the passivation layer 3206 to form the hole 3202 may be reactive-ion etching, which may result in the sidewall surface of the hole 3202 having a vertical profile. At (g), the photoresist 3502 and a portion of the Ti/Pt layer 3228 on top of the photoresist 3502 may be removed in a lift-off process, such that the Ti/Pt layer 3228 coating the bottom and sidewall surfaces remains. At (h), a layer of PtB 3230 may be formed on the Ti/Pt layer 3228 in the hole 3202 using an electrodeposition technique. In some embodiments, cyclic voltammetry may be used to form the layer of PtB 3230, which may result in the layer of PtB 3230 having a bulbous or rounded profile at an outer rim or edge of the hole 3202, as schematically depicted in FIG. 35b at (h).

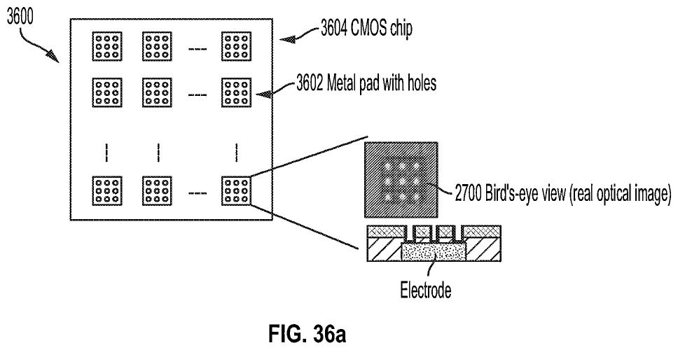

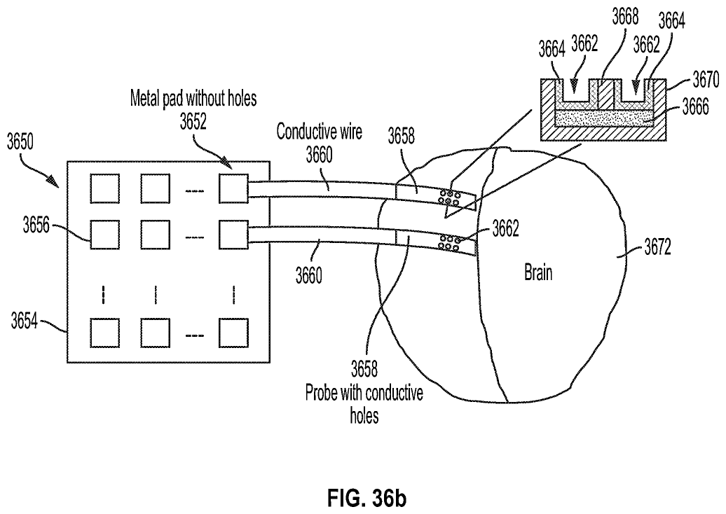

FIGS. 36a and 36b schematically show illustrations of applications that may use hole arrays, according to some embodiments of the technology disclosed herein. A hole-array device 3600 that may be used for in vitro or ex vivo applications is shown schematically in FIG. 36a. In some embodiments, the device 3600 may be comprised of a plurality of electrodes 3602 fabricated on a single CMOS chip 3604. Each electrode 3602 may be comprised of at least one hole array (e.g., the hole array 2700), as depicted in the bird's-eye or plan view and the cross-sectional view in the inset of FIG. 36a. (See also FIG. 27b at (e′).) More specifically, as discussed herein, each hole array-type electrode may be comprised of a metallic pad (electrode pad) and a plurality of conductive holes disposed on a surface of the metallic pad and structured to increase the electrically conductive contact area of the electrode 3602 relative to the electrically conductive contact area of nanowire-type electrodes. The electrodes 3602 may be arranged in a regular pattern of columns and rows or may have any suitable arrangement that can be fabricated using CMOS technologies. In some embodiments, each of the electrodes 3602 may be operably connected to circuitry (not shown) disposed on the chip 3604. The circuitry may be configured to record signals from the electrode 3602 and/or to provide stimulation signals to the electrode 3602. In operation, the device 3600 may operate by exposing the holes of the electrodes 3602 to a specimen to be investigated (e.g., one or more biological cell(s)), such that the specimen may physically contact recesses corresponding to the holes and, in some cases, at least partially conform with the recesses such that the recesses are at least partially filled by the specimen. As discussed above, the conductive surfaces of the holes may increase the surface area in contact with the specimen, which may be beneficial for accurately recording signals from the specimen, even small-amplitude signals of less than 0.5 mV.

A hole-array device 3650 that may be used for in vivo applications is shown schematically in FIG. 36b. In some embodiments of the present technology, the device 3650 may be comprised of a plurality of electrodes 3652 fabricated partially on a single CMOS chip 3654 and partially off-chip. More specifically, each electrode 3652 may be comprised of a metallic pad 3656 connected to an off-chip probe 3658 by conductive wiring 3660. The probe 3658 may be comprised of a plurality of conductive holes 3662, which may be similar to the holes 2755 discussed above (see FIG. 27b). Similar to the device 3600, the metallic pads 3656 may be formed on the chip 3654 (e.g., by CMOS technology) and may be operably connected to circuitry (e.g., CMOS circuitry, not shown) disposed on the chip 3654. The circuitry may be configured to record signals from the electrode 3652 and/or to provide stimulation signals to the electrode 3652. In some embodiments, each hole 3662 of the probe 3658 may be structured as shown in the inset of FIG. 36b, which schematically depicts a cross section through a pair of holes 3662. It should be understood that the holes 3662 of the probe 3658 may have an arrangement other than the 2×3 arrangement shown in FIG. 36b. In some embodiments, external surfaces of the holes 3662 may be comprised of a layer of a conductive material 3664. For example, the conductive material 3664 may be a layer of PtB on sidewalls and bottoms of the holes 3662. The bottoms of the holes 3262 may be in physical contact with a conductive base 3666, which in turn may be electrically connected to the metallic pad 3656 via the wiring 3660. The holes 3662 of each probe 3658 may be electrically connected to each other via the conductive base 3666. The holes 3662 of each probe 3658 may be separated from each other by an insulation layer 3668, which may be similar to the insulation layer 2756 discussed above (see FIG. 27b). A passivation layer 3670 may surround a periphery of the conductive material 3664 and may serve to isolate an outer periphery of the holes 3662 of the probe 3658. The passivation layer 3670 also may surround an outer surface of the wiring 3660. In some embodiments, the insulation layer 3668 and the passivation layer 3670 may be interconnected and may be formed of a same material. In some embodiments, the device 3650 may operate by having the holes 3662 of the probes 3658 contact a live specimen to be investigated, such as a brain 3672 of a patient being studied. Portions of the brain 3672 that are in contact with the holes 3662 may at least partially conform with the holes 3662 such that the holes 3662 are at least partially filled by the portions of the brain 3672. As discussed above, the conductive surfaces of the holes 3662 may increase the surface area in contact with the brain 3672, which may be beneficial for accurately recording small neuronic and other signals from the brain 3672, which may have amplitudes in a range of about 2 μV to 100 mV.

According to some embodiments of the present technology, the holes 3662 of the probes 3658 may be fabricated by a process similar to that described above in connection with FIG. 27b. In some embodiments, the holes 3662 of the probes 3658 may be fabricated or by a process similar to that described above in connection with FIG. 35b. In some embodiments, instead of a plurality of holes, one or more of the probes 3658 may be comprised of a single hole.

It should be understood that the scope of the disclosure of the present technology may include processes for fabricating the probe 3658 and/or the hole arrays 2700, 3200 that may be modifications of the processes described herein.

The disclosure of the present technology, aspects of which are described herein, provides electrode structures that may be able to provide recording characteristics that may be superior to those of known electrode structures. In some embodiments of the present technology, the electrode structures may be fabricated more simply while providing improved characteristics. As can be appreciated from a comparison of FIG. 27b with FIG. 27a at process A, electrodes comprised of hole arrays may be fabricated with fewer than half the number of procedures typically used for fabricating electrodes comprised of nanowires, and, additionally, the hole arrays may provide a greater surface area for contacting a specimen to be investigated. Moreover, the hole arrays may be used in conjunction with the feedback-based stimulation techniques described herein, in which injection of DC stimulation currents to the electrodes may be toggled on and off during recording, as discussed above in connection with FIG. 31. The disclosed feedback-based stimulation in conjunction with the hole arrays has been found to permit recording of signals for prolonged durations in excess of 2 hours and, in some cases, in excess of 3 hours. The feedback-based techniques of the present technology has demonstrated a breakthrough in the field of parallel intracellular recording in terms of number of coupled neurons and the coupling duration (see FIGS. 30a through 30e), making the use of electrodes comprised of CMOS-fabricated hole arrays more appealing to a wider range of communities, particularly neuroscientists, than other types of electrodes currently in use. In some embodiments of the present technology, the electrode structures and measurement schemes disclosed herein may minimize current leakage for measurements of cells in solution, as discussed above in connection with FIG. 34.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the invention may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within +20% of a target value in some embodiments, within +10% of a target value in some embodiments, within +5% of a target value in some embodiments, and yet within +2% of a target value in some embodiments. The terms “approximately” and “about” may include the target value.

### Further Examples of a Semiconductor Device for Electrically Assessing Cells or Other Biological Specimens

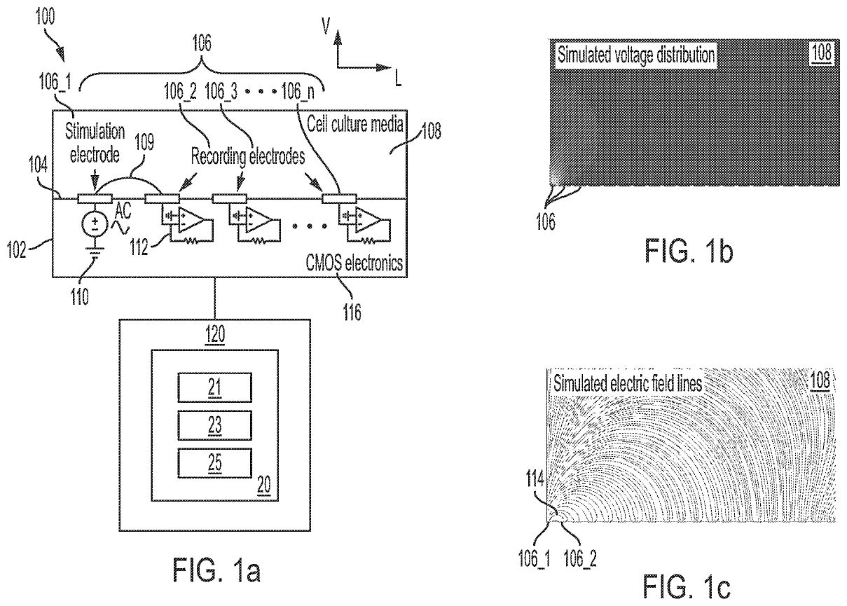

FIG. 1a is a schematic diagram of a side view of an apparatus 100 formed on a semiconductor substrate 102, in accordance with some embodiments of the present technology. In some embodiments, the apparatus 100 may be comprised of an electrode array 106 that includes a plurality of electrodes 106_1, 106_2, 106_3, . . . , 106_n disposed on a surface 104 of the semiconductor substrate 102. FIG. 1a also illustrates an example of a cross-electrode impedance measurement, which may be performed by applying a voltage stimulus to a first electrode 106_1 and measuring a current at a second electrode 106_2. The measured current, which may be referred to as a cross-electrode current between the electrodes 106_1, 106_2, flows along one or more current paths 109 in a medium 108 that is in contact with the electrode array 106. The first electrode 106_1, which may be connected to a stimulus-source circuit 110, may be referred to as a stimulation electrode. The second electrode 106_2, which may be connected to a current-measuring circuit 112, may be referred to as a recording electrode. In some embodiments, the current-measuring circuit 112 may be referred to as a recording circuit 112.

A cross-electrode impedance between the first and second electrodes 106_1, 106_2 may be obtained from values of a cross-electrode current and a stimulus voltage between these electrodes using any suitable method known in the art (e.g., by dividing an amplitude of the stimulus voltage with an amplitude of the cross-electrode current). A processing unit 120 may be provided that receives signals from active circuitry within the semiconductor substrate 102 and that performs a determination of the cross-electrode impedance. It should be appreciated that there is no requirement to calculate actual impedance values, and that any representative measurement that is indicative of impedance between the first and second electrodes 106_1, 106_2 may be used. Alternatively or in addition to be used to calculate a value for the cross-electrode impedance, the cross-electrode current may be used as an indicator for the cross-electrode impedance when measurements at different electrodes are compared, if the amplitude of the stimulus voltage is programmed to be a known constant.

FIG. 1b is a two-dimensional chart plotting a simulated voltage distribution in the apparatus 100 shown in FIG. 1a, and shows that when a voltage is applied to the stimulation electrode 106_1 a potential in the medium 108 may fall off both along a vertical direction (V) and a lateral direction (L) away from the stimulation electrode 106_1. FIG. 1c is a chart plotting simulated electric field lines 114 corresponding to the chart shown in FIG. 1b. As shown in FIG. 1c, the electric field lines 114 emanate from the stimulation electrode 106_1 in directions that are upward (i.e., vertical) and outward (i.e., lateral) from the stimulation electrode 106_1, that curve laterally toward the recording electrodes 106_2, 106_3, . . . , 106_n of the apparatus 100, and that subsequently are directed downward to terminate at the recording electrodes 106_2, 106_3, . . . , 106_n. The electric field lines 114 have vertical components and lateral components. The term “vertical field” may be used herein to refer to an electric field's vertical component. Similarly, the term “lateral field” may be used herein to refer to an electric field's lateral component.

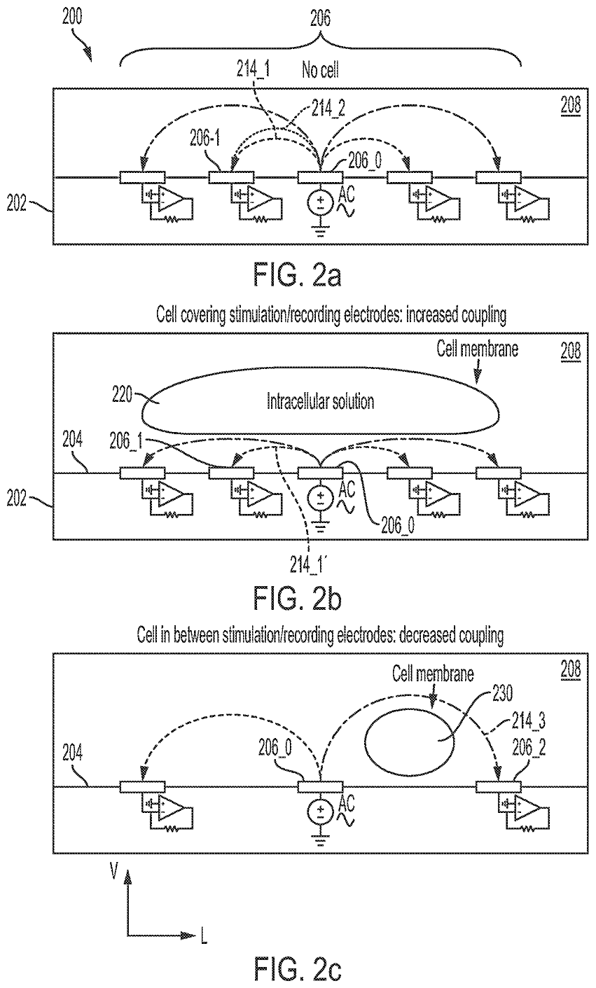

The presence of cells may alter the shape and distribution of the electric field lines 114 between the stimulation electrode 106_1 and the recording electrodes 106_2, 106_3, . . . , 106_n and in turn may lead to a change in cross-electrode coupling, as discussed in detail below. FIG. 2a is a schematic diagram of a side view of an apparatus 200 comprised of a semiconductor substrate 202 without a cell present, in accordance with some embodiments of the present technology. In FIG. 2a, an electrode array 206 may be comprised of an electrode 206_0 configured to be a stimulus electrode, with electric fields lines 214_1, 214_2 linking the stimulus electrode 206_0 and a recording electrode 206_1. FIG. 2b is a schematic diagram of a side view of the apparatus 200, illustrating a scenario in which a cell 220 in a solution 208 is disposed over some electrodes of the electrode array 206 of FIG. 2a. FIG. 2c is a schematic diagram of a side view of the apparatus 200, illustrating a scenario in which a cell 230 in the solution 208 is not disposed over the electrode array in FIG. 2a but instead is disposed in between the stimulus electrode 206_0 and a recording electrode 206_2.

The inventors have recognized and appreciated that a biological cell may have a lipid bilayer that forms a continuous membrane barrier around the cell. Electrically, the membrane can behave as a capacitor in parallel with a high resistance, and can have a different electrical impedance compared to an impedance of a surrounding medium such as a solution containing the cell. A cell with its high-impedance membrane positioned on top of an electrode array (e.g., the electrode array 206) may affect the current distribution in the solution containing the cell (e.g., the solution 208) by blocking a vertical path of the field lines from the stimulus electrode to the recording electrode(s). FIG. 2c depicts the cell 230 suspended in the solution 208 such that the cell 230 blocks a lateral path of the field lines in the solution 208 and lowers a nearest-neighbor coupling between the stimulus electrode 206_0 and the recording 206_2. That is, the field lines traverse a longer vertical distance to go around the cell 230 to reach the recording electrode 206_2 from the stimulus electrode 206_0, in comparison to the field lines of FIG. 2a where no cell is present. In contrast, as depicted in FIG. 2b, the cell 220 may be disposed above the surfaces of the electrode array 206 and may be positioned on top of the surface of the stimulus electrode 206_0 and the surface of the recording electrode 206_1, such that cross-electrode coupling is increased between the electrodes 206_0, 206_1. That is, the presence of the cell 220 may deter or block the field lines from traversing vertically thus causing the field lines to be shorter and closer to a surface 204 of the apparatus 200.

As an example for illustrating the effect of the presence of cells on cross-electrode coupling, and without wishing to be bound by a particular theory, the inventors recognized that if a cell such as the cell 220 in FIG. 2b is adhered to the surface 204 of the apparatus 200 such that the cell 220 is above some of or an entirety of the stimulus electrode 206_0 and the recording electrode 206_1, the cell 220 may increase the cross-electrode coupling by blocking the electric field lines 214_1, 214_2 (see FIG. 2a) between the electrodes 206_0, 206_1 from extending vertically into the solution 208 and therefore confining the electric field lines 214_1, 214_2 to be closer to the surface 204 of the apparatus 200. As a result, the outer field line 214_2 in FIG. 2a (where no cell is adhered) may be suppressed in FIG. 2b due to the presence of the cell 220, and the inner field line 214_1 in FIG. 2a may be a strengthened field line 214_1′ in FIG. 2b, resulting in a stronger coupling and a lower impedance between the electrodes 206_0 and 206_1 when the cell 220 is adhered. It should be understood that the term “adhered” may refer to the cell 220 being disposed vertically above the electrodes 206_0, 206_1 without physically touching the electrodes 206_0, 206_1. That is, there may be a non-zero distance separating the cell 220 and the electrodes 206_0, 206_1.

On the other hand, if a cell is not adhered to the electrode array 206 but instead is disposed laterally relative to the stimulus electrode 206_0 and one or more of the recording electrodes 206_1, 206_2, such as the cell 230 in FIG. 2c positioned laterally between the stimulus electrode 206_0 and the recording electrode 206_2, the cell 230 may lengthen a path of an electric field line 214_3 between the electrodes 206_0, 206_2 relative to when no cell is present (see FIG. 2a), thus decreasing cross-electrode coupling between the electrodes 206_0, 206_2 and increasing the impedance between the electrodes 206_0, 206_2.

Therefore, the presence of a cell (e.g., the cell 220 or the cell 230) in the solution 208 near the electrode array 206 and whether the cell is adhered to the electrode array 206 (e.g., the cell 220) or is disposed between the stimulation electrode and one or more of the recording electrodes (e.g., the cell 230) may be detected using cross-electrode coupling measurements.

An advantageous aspect of cross-electrode coupling measurements such as those described in connection with various embodiments of the present disclosure is that they may permit non-invasive measurements to be performed repeatedly without affecting cell viability and/or without degrading measurement capabilities of the electrodes. It should be understood that a cell that is adhered to an electrode may have various degrees of non-zero separation between an outer extent or periphery of the cell membrane and the surface of the electrode. An apparatus according to some aspects of the present technology may provide detection for a degree to which a cell is adhered. For example, a relatively stronger adhesion may be determined from a relatively stronger or greater increase in cross-electrode coupling due to a smaller gap or vertical distance between the cell and the surface of the electrode.

As described above in connection with FIG. 2b, cross-electrode coupling techniques according to some embodiments of the present technology may be used to detect and measure an increase in coupling between electrode pairs due to suppression of vertical components of electric field lines caused by the presence of a cell. Measurement of an increased cross-electrode coupling (or a decreased cross-electrode impedance) to determine the presence of a cell, such as described above, may provide advantages over measurement of an increased impedance to determine the presence of a cell. One advantage for using an increase in cross-electrode coupling as an indicator to detect cell presence is that such an increase may be attributed mainly to electrode pairs that are close to each other, in some cases to nearest-neighbor coupling between the electrode pairs (e.g., the stimulus electrode 206_0 and an adjacent recording electrode). Therefore, a measured increase in cross-electrode coupling (or a decrease in cross-electrode impedance) between the stimulus electrode 206_0 and, e.g., the recording electrode 206_1 can be separated from a total background current flowing through the stimulus electrode 206_0 other electrodes in the electrode array 206. As a result, signal-to-background ratio and sensitivity of cell detection can be improved.

In contrast to cross-electrode measurement techniques, some embodiments of which are disclosed herein, the inventors have recognized that simple impedance measurements, which are measured at individual electrodes (e.g., at individual recording electrodes), may fail to detect the presence of cells. In such a simple impedance measurement on one electrode, a sum of all return currents may be measured as a signal for the electrode's impedance. That is, such a simple measurement is an impedance measurement of the electrode only, and does not measure a change in the electric field in the environment (e.g., the solution 208) surrounding the electrode. As a result, the inventors have observed that an impedance of an electrode itself, as determined from a simple impedance measurement, is not sensitive to the presence of a cell even if the cell is positioned directly on the electrode's surface. In comparison, a cross-electrode measurement between a pair of electrodes, according to embodiments of the present technology, may provide enhanced sensitivity and may be used to determine the presence of a cell and whether the cell is positioned above the pair of electrodes or between the pair of electrodes.

Referring back to FIG. 1a, in some embodiments of the present technology, a stimulus signal applied by the stimulus source circuit 110 to the stimulus electrode 106_1 may be a low-frequency alternating current (AC) signal having a frequency of less than 10 kHz, or less than 5 kHz, or between 0.1 kHz and 5 kHz, or between 0.1 kHz and 2 kHz. The low-frequency AC stimulus signal may be used instead of a DC stimulus signal because a cell's membrane may act as a capacitor in parallel with a high resistance, and, at a high frequency, the capacitor's impedance would decrease and render the cell highly conductive. The inventors have recognized and appreciated that by measuring a cross-electrode current at a low frequency a high signal contrast may be observed for detection of an adhered cell above a pair of electrodes (e.g., the cell 220) or a cell disposed laterally relatively to the pair of electrodes (e.g., the cell 230). An example of a frequency response of a cross-electrode impedance measurement is provided below in Example 4.

Still referring to FIG. 1a, the semiconductor substrate 102 may include active circuitry 116, which may be comprised of CMOS electronics. According to some embodiments of the present technology, the active circuitry 116 may include a plurality of stimulus circuits 110 and a plurality of recording circuits 112. In some embodiments, a stimulus circuit 110 may comprise one or more current injectors, one or more voltage sources, or a combination thereof. In some embodiments, aspects of the active circuitry 116 may relate to current-based stimulators for electrogenic cells and related methods, as disclosed in International Publication No. WO 2019/010343 for International Patent Application No. PCT/US2018/040969 entitled “CURRENT-BASED STIMULATORS FOR ELECTROGENIC CELLS AND RELATED METHODS,” the disclosure of which is hereby incorporated by reference in its entirety. In some embodiments, the active circuitry 116 may be comprised of programmable current injectors for performing current-voltage measurements using one or more of the electrodes of the electrode array 106 as working and/or counter electrodes. In some embodiments, aspects of the apparatus 100 may relate to electronic circuits for analyzing electrogenic cells and related methods, as disclosed in International Publication. No. WO 2019/089495 for International Patent Application No. PCT/US2018/058081 entitled “ELECTRONIC CIRCUITS FOR ANALYZING ELECTROGENIC CELLS AND RELATED METHODS,” the disclosure of which is hereby incorporated by reference in its entirety.

According to some embodiments of the technology disclosed herein, each recording circuit 112 may include a transimpedance amplifier (TIA) having a switching capacitor as an impedance component. In some embodiments, the impedance component of the TIA may have a resistance of at least 10 MO (megohms), or at least 100 MΩ, or between 10 MΩ and 1 GΩ (gigaohm), to provide amplification of a recorded current signal at an input of the TIA. The TIA may provide an output voltage that is proportional to the recorded current signal and to a voltage across the impedance component.

Electrodes in the electrode array 106 may be reconfigured using the active circuitry 116, such that an electrode may be reconfigured to be a stimulation electrode or a recording electrode, according to some embodiments of the present technology. In some embodiments, the active circuitry 116 may be comprised of routing and switching components that may be programmable and configured to connect a selected electrode of the electrode array 106 to a stimulus circuit 110, and/or to a current-measuring circuit 112, and/or to other circuit components to enable different functionalities. Depending on how the electrode array 106 is to be used, more than one electrode may each be configured as a stimulus electrode, and more than one electrode may each be subjected to recording at the same time. For example, in some embodiments, for an application that involves mapping local cell properties using cross-electrode impedance measurements, typically only one electrode may act as a stimulus electrode at a time. In some other embodiments, a subset of electrodes may be selected to act as stimulus electrodes to apply one or more potentials or one or more currents to initiate electrochemical reactions at the locations of the selected subset of electrodes. The latter embodiments will be discussed in more detail in the sections below regarding cell-to-cell attachment measurement, patterning, and spatial electrochemical mapping of cells.

According to some embodiments of the technology disclosed herein, electrodes in the electrode array 106 may be biased using low-impedance sources/returns in the active circuitry 116. For example, a low output-impedance voltage source may be used to provide a stimulus signal at a stimulus electrode, while a low input-impedance TIA may be provided for current measurement at a recording electrode. In some embodiments, each electrode may be selectively connected to a voltage source for stimulation, to a TIA for current measurement and/or a voltage source for a return, or to a TIA for simultaneous stimulation and current measurement. The inventors have appreciated and recognized that a low-impedance source/return may facilitate formation of fringing electric field lines in an environment of the electrode array 106 (e.g., in the medium 108), an example of which is illustrated in FIG. 1c.

According to some embodiments of the present technology, the semiconductor substrate 102 may be comprised of silicon. In such embodiments, the active circuitry 116 may be an integrated circuit that comprises CMOS components fabricated using standard CMOS processing techniques. The electrode array 106 may be disposed within and/or on the semiconductor substrate 102. For example, portions of the electrode array 106 may be comprised of conductors exposed at the surface 104 of the semiconductor substrate 102 such that the conductors face the medium 108. In some embodiments, the surface 104 may be an insulative surface that provides mechanical support and electrical isolation to the electrode array 106 while also providing a suitable surface for cells to grow. Although FIG. 1a shows the electrode array 106 to be partially embedded in the semiconductor substrate 102, it should be understood that such an arrangement is an illustrative example only and not a requirement. In some embodiments, top surfaces of electrodes in electrode array 106 may be above, aligned vertically with, or below the surface 104 of the semiconductor substrate 102. Additionally or alternatively, the top surfaces of the electrodes may have a passivation layer or functionalization layer. In some embodiments, holes may be patterned in the passivation or functionalization layer on top of an electrode to expose the conductive surface of the electrode to the medium. In some embodiments, the holes may be comprised of conductive surfaces that are electrically connected to the electrode, as discussed herein.

According to some embodiments of the present technology, the semiconductor substrate 102 may be any substrate that may be fabricated and/or processed using semiconductor processing techniques, and is not limited to being a silicon wafer. For example, the semiconductor substrate 102 may be comprised of a group-IV semiconductor, a III-V semiconductor, II-VI semiconductor, an sp² hybridized carbon material, a chalcogenide, a metal, a metallic compound, an oxide, a nitride, a silicide, a polymer material, or combinations thereof. The semiconductor substrate 102 may include a unitary component or a composite of multiple components. For example, components in the semiconductor substrate 102 may be comprise of an active circuit layer, a wiring layer, a redistribution layer, a circuit board, or combinations thereof. In some embodiments, component layers in the semiconductor substrate 102 may be formed via an additive process during CMOS processing or may be formed separately and bonded to each other using packaging techniques known in the field of semiconductor processing. Conductors may be provided in the semiconductor substrate 102 that interconnect the active circuitry 116 with the electrode array 106. In some embodiments, connection points may be provided at a bottom surface of the semiconductor substrate 102 for electrically interfacing components in the semiconductor substrate with the processing unit 120. In some embodiments, an electrical connection for communications between the processing unit 120 and the semiconductor substrate 102 may be provided via any suitable way, such as but not limited to any one or any combination of: controlled collapse chip connection (also known as C4 or flip-chip bonding), wire bonding, flexible cables, and wireless communication techniques.

According to some embodiments of the present technology, the apparatus 100 may be operated to perform various methods. For example, the apparatus 100 may be used for mapping reactions of an object being investigated, for performing selective electrochemistry, etc. Operation of the apparatus 100 may be electronically controlled by a computer program. In some embodiments, the processing unit 120 may comprise a computer 20 operably connected to a storage device 21, a memory device 23, and a processor 25. Processing or execution of the computer program may be performed in the computer 20 or any other computing device operably connected to the computer 20. The storage device 21 and the memory device 23 may be comprised of one or more suitable non-transitory computer-readable medium, non-limiting examples of which include: a hard-disk memory, a compact disc, an optical disc, a magnetic tape, a flash memory, circuitry for a Field Programmable Gate Array (FPGA) or another semiconductor device, and other tangible storage media able to store electronic data. In some embodiments, the storage device 21 may be comprised of a non-volatile storage device (e.g., a ROM device), and the memory device 23 may be comprised of a volatile storage device (e.g., a RAM device). In some embodiments, computer-executable instructions may be loaded from the storage device 21 to memory device 23 before execution by the processor 25. When executed, the computer-executable instructions may cause the processor 25 to perform some of or all of the methods described herein. However, it should be understood that a distinction between the storage device 21 and the memory device 23 is not critical and either or both may be present in some embodiments.

According to some embodiments of the technology disclosed herein, the processor 25 may be any suitable processing device, non-limiting examples of which include one or more of the following: a processor, a central processing unit (CPU), a digital signal processor (DSP), a controller, a addressable controller, a special purpose microprocessor, a microcontroller, an addressable microprocessor, a programmable processor, a programmable controller, a dedicated processor, a dedicated controller, and any other suitable processing device. In some embodiments, the processing unit 120 may be partially or wholly packaged as a system-on-a-chip (SOC). Moreover, it should be appreciated that FIG. 1a shows a schematic representation of the processing unit 120. In some embodiments, an actual implementation of the processing unit 120 may have a system configured to perform distributed processing. A host computer, for example, may control an overall flow of a procedure that includes obtaining measurement data, mapping data results, and analysis of data results.

Turning now to the electrode array 106, according to some embodiments of the technology disclosed herein, the electrode array 106 may be patterned on the surface 104 of the semiconductor substrate 102 as part of the semiconductor fabrication process to form the active circuitry 116 in semiconductor substrate 102. The electrode array 106 may be comprised of conductive pads that include metal (e.g., Au, or Pt, or alloys thereof). In some embodiments, the pads may be formed of Al with plated Au as a top layer. In some embodiments, the substrate 102 may additionally comprise conductors that interconnect vertically and laterally the electrode array 106 exposed on the surface 104 to the active circuitry 116 in the substrate 102.

According to some embodiments of the present technology, electrodes of the electrode array 106 may be arranged on the surface 104 in any suitable arrangement, such as a two-dimensional array with regular pitches along row and column directions. In some embodiments that may involve cross-electrode impedance-based mapping, a pitch of the electrode array may be selected to be on the order of or smaller than a size of typical cells such that a cell can cover at least two electrodes, to increase the occurrence of coupling between the cell and the at least two electrodes. For example, for a cell size of about 30 μm, the pitch of the electrode array 106 may be set at less than 30 μm, or less than 20 μm, or less than 5 μm, or between 1 and 20 μm. By providing a smaller pitch between electrodes relative to the cell size, a likelihood of a cell being able to cover or adhere to two or more electrodes is increased, which may increase cross-electrode coupling of the electrodes under the cell and facilitate measurements of cell-to-substrate gap distance.

According to some embodiments of the present technology, the electrode array 106 may be fabricated during a CMOS-compatible fabrication process on top of the semiconductor substrate 102 after the active circuitry 116 has been formed in the substrate 102 by CMOS-based processes. A pitch of the electrode array 106 and a size of each electrode may be selected by taking into consideration pitch and density of the active circuitry 116. For example, in some embodiments, at least 8 recording circuits 112, or at least 10 recording circuits 112, or at least 4000 recording circuits 112 may be provided in the semiconductor substrate 102 as part of the active circuitry 116, and the electrode array 106 may include at least 1000 electrodes, or at least 4000 electrodes, or at least 1,000,000 electrodes. In some embodiments, each electrode may have a lateral dimension of no more than 10 μm, or no more than 5 μm, such that an overall lateral extent of the electrode array 106 may be contained within the surface of a single chip comprising the semiconductor substrate 102. In some embodiments, the electrode array 106 may be a CMOS microelectrode array (MEA) and may be referred to as a MEA 106.

Referring back to FIG. 1a, the medium 108 may be a cell culture medium and may be comprised of a solution that includes any number of chemical and/or biological reagents in addition to cells, according to some embodiments of the present technology. Although not shown in FIG. 1a, the medium 108 may be contained in a container sealed to and disposed on top of the semiconductor substrate 102. In some embodiments, the container may be a well of a multiple-well plate sealingly attached to the semiconductor substrate 102, with one or more wells having an open bottom exposing contents of the one or more wells to the semiconductor substrate 102. In some embodiments, the electrode array 106 may be one of multiple electrode arrays of the semiconductor substrate 102 that are exposed to multiple wells, respectively, thus permitting multiple assessments to be conducted in parallel in the multiple wells.

Various embodiments of the present technology may be used to provide CMOS-compatible, wafer-scale, multiple-well platforms, which may be utilized for biomedical or other applications. In some embodiments, circuitry may be provided underneath a multiple-well array to electrically interface with electrodes in the wells. A platform that operates with multiple wells may sometimes be referred to as a CMOS-Multiwell Platform. The inventors have recognized and appreciated that to interface with electrodes in a large array, circuitry may be fabricated on a single semiconductor chip (e.g., a silicon (Si) wafer or chip) having a dimension that is at least equal to or larger than that of the multiple-well array. According to one aspect of the present disclosure, standard CMOS fabrication processes such as those known to be used in a standard semiconductor foundry may be used, e.g., without expensive customization for complex fabrication procedures, and thus production costs can be controlled in some cases. In some embodiments, the CMOS-Multiwell Platform may be used in applications including, e.g., electrophysiology studies and general cell assessments using electrical methods and/or electrochemical methods. In some embodiments, a high throughput format may be used (e.g. a 24-well plate format, a 96-well plate format, a 384-well plate format, etc.).

According to some embodiments of the present technology, a Si wafer may be part of a semiconductor device that includes a plurality of reticle areas each corresponding to an apparatus 100 comprised of multiple electrodes. The reticle areas may be arranged as an array of reticle areas, with some or all of the reticle areas having circuitry of a same design. The inventors have recognized and appreciated that during manufacturing, reticle areas of a wafer may reuse the same lithographical mask design repeated across the Si wafer, which in some cases may reduce tooling costs and may increase wafer manufacturing throughput for a device comprised of multiple apparatuses 100. In some embodiments, digital and/or analog circuitry may be included within a reticle area and may be arranged to correspond to one or more wells when a multiple-well array is coupled to the Si wafer (e.g., on top of the wafer). Some embodiments may permit wafer-scale integration of an electrical interface with a multiple-well array by using a manufacturing method that does not dice the Si wafer into separate chips each include a reticle area.

Some embodiments of the present disclosure may provide techniques for mapping a spatial distribution and dimensions of cells using cross-electrode impedance measurements. The mapping may additionally represent a property of individual cells, e.g., a property relating to a degree to which individual cells adhere to electrode surfaces. In some embodiments, because cell presence may be reflected primarily by local cross-electrode coupling between a stimulus electrode and a set of nearby recording electrodes of an electrode array, the mapping may be performed by choosing an individual electrode to be the stimulus electrode and then obtaining a set of cross-electrode data for the recording electrodes nearby the stimulus electrode, such as by measuring a cross-electrode impedance between the stimulus electrode and each of the nearby recording electrodes of the electrode array. Subsequently, a different electrode may be chosen to be the stimulus electrode, and a different set of nearby recording electrodes may be measured to obtain of a different set of cross-electrode data for the electrode array. Such cross-electrode measurements may be repeated for some or all of the electrode array by sequentially setting different electrodes in the electrode array to apply a stimulus signal (i.e., to be the stimulus electrode) and obtaining a corresponding set of data of cross-electrode measurements of nearby recording electrodes. The data may then be processed to generate a value that indicates, for each stimulus-electrode location, whether there is a presence of a cell and, if so, a strength of a cell property. The generated values may then be combined to form a map representing some or all of the electrode array. In some embodiments, “electrochemical imaging” of live-cell cultures may be performed by high-resolution in situ cross-electrode impedance measurements and electrochemical measurements. In some embodiments, CMOS-based MEAs may be used to perform label-free and non-invasive tracking of cell growth dynamics and to obtain accurate measurements of cell-substrate attachment or adherence, cell-cell adhesion, and metabolic state.

Some embodiments of the present disclosure may provide techniques for spatially positioned electrochemical reactions using an electrode array exposed on a surface of a semiconductor substrate. For selected electrodes in the electrode array in a solution, active circuitry in the semiconductor substrate may apply potentials to initiate an electrochemical reaction in regions of the solution directly above the selected electrodes. For example, the active circuitry may be programmed to apply potentials to a selected group of electrodes of the electrode array, e.g., based on a size of the selected group, and/or a shape of the selected group, and/or a distribution of the selected group. In some embodiments, spatially programmed electrochemistry may be used to perform cell patterning. For example, cells adhered to an electrode may be selectively removed from the electrode surface by electrochemically generated small gas bubbles on the electrode. In some embodiments involving electrochemistry, the electrode array may be used to spatially map analyte concentrations as measured using the active circuitry in the semiconductor substrate. For example, such electrochemical mapping of solutions may utilize redox electrochemistry.

Each of the following publications and applications is incorporated herein by reference in its entirety: International Publication. No. WO 2021/257705 for International Patent Application No. PCT/US2021/037630 entitled “SYSTEMS AND METHODS FOR PATTERNING AND SPATIAL ELECTROCHEMICAL MAPPING OF CELLS,” which claims the benefit of priority of U.S. Provisional Patent Application No. 63/040,439 filed Jun. 17, 2020; International Publication. No. WO 2021/257701 for International Application No. PCT/US2021/037626 entitled “APPARATUSES FOR CELL MAPPING VIA IMPEDANCE MEASUREMENTS AND METHODS TO OPERATE THE SAME,” which claims the benefit of priority of U.S. Provisional Patent Application No. 63/040,424 filed Jun. 17, 2020; and International Publication. No. WO 2021/257686 for International Patent Application No. PCT/US2021/037604 entitled “COMPLEMENTARY METAL-OXIDE-SEMICONDUCTOR (CMOS) MULTI-WELL APPARATUS FOR ELECTRICAL CELL ASSESSMENT,” which claims the benefit of priority of U.S. Provisional Patent Application No. 63/040,412 filed Jun. 17, 2020.

The examples presented below are intended to illustrate various embodiments of the inventive technology disclosed herein. These examples do not represent the full scope of the inventive technology.

### Example 1: Real-Time Cell Measurements Using a CMOS Microelectrode Array (MEA) and Imaging System

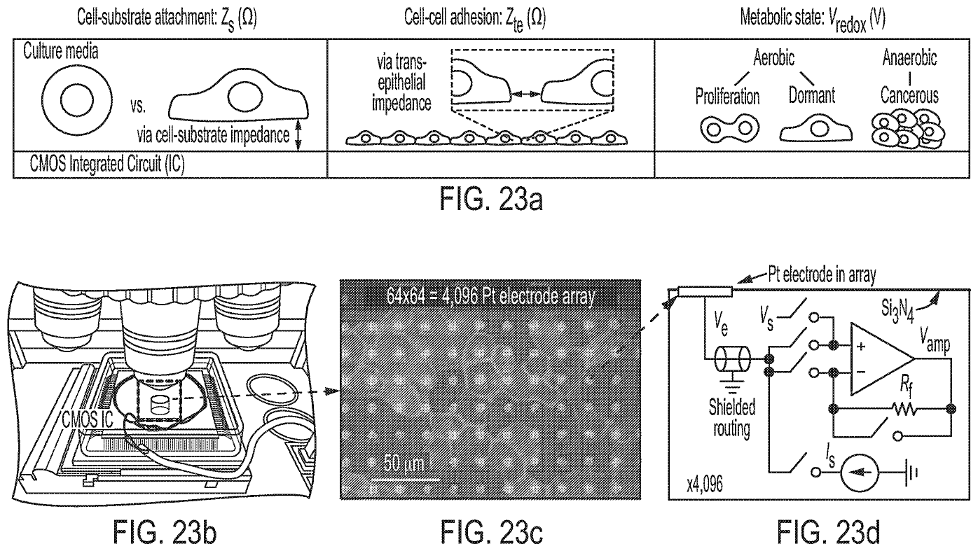

This example describes techniques involving electrical imaging of three parameters useful for cell assessment of live cells. FIG. 23a schematically shows: cell-substrate impedance, Zₛ, which reflects cell attachment and cell-substrate adhesion; transepithelial impedance, Zₜₑ, which reflects cell-cell adhesion and an integrity and barrier function of a cell monolayer; and a extracellular redox potential, V_{redox}, which reflects respiration and a cellular metabolic state, which are discussed in more detail below.

In this example, a CMOS IC device is used that parallelizes impedance and electrochemical measurements across a 64×64=4,096 array of electrodes. FIG. 23b illustrates an imaging and measurement arrangement in which the device, which is comprised of a fluidic well packaged on top of a CMOS chip configured to culture cells, is mounted below a top-down fluorescence microscope for simultaneous optical and electrical measurements. FIG. 23c shows an image of a portion of the array of electrodes. The array of electrodes sits on the surface at the center of the device and is comprised of 8-μm diameter Pt electrodes spaced at a 20-μm pitch for single- or few-cell resolution (e.g. Madin-Darby Canine Kidney (MDCK) cells, which are depicted in FIG. 23c), and results in a total sensing area of 1.26×1.26 mm². A remainder of the surface is insulated with silicon nitride, which has characteristics similar to those of glass culture plates. No difference was observed in growth or morphology for cells cultured by the device in comparison with traditional culture plates. For long-term measurements (e.g., for durations greater than about 1 hour, or 2 hours, or 3 hours), an integrated temperature sensor and a heater may be used to regulate the cells to temperatures in a range of 35° C.-37° C. and a mini-incubation chamber may be placed over the device to regulate CO₂ to 5% of the atmospheric environment.

As depicted in FIG. 23d, each electrode in the array is part of its own pixel and is connected to its own pixel circuit, which is highly configurable and programmed via a digital interface. The pixel circuit is comprised of an operational amplifier, which can be configured as a buffer for an electrode voltage, Ve, measurement, or as a TIA for electrode current, Iₑ, measurement. In some embodiments of the present technology, the pixel circuit may be configured to include current-based stimulators for electrogenic cells and/or to use related methods, as described in International Publication No. WO 2019/010343 for International Patent Application No. PCT/US2018/040969 entitled “CURRENT-BASED STIMULATORS FOR ELECTROGENIC CELLS AND RELATED METHODS,” the disclosure of which is hereby incorporated by reference in its entirety. Some aspects of this example may utilize electronic circuits for analyzing electrogenic cells and/or may use related methods, as described in International Publication. No. WO 2019/089495 for International Patent Application No. PCT/US2018/058081 entitled “ELECTRONIC CIRCUITS FOR ANALYZING ELECTROGENIC CELLS AND RELATED METHODS,” the disclosure of which is hereby incorporated by reference in its entirety.

In FIG. 23a, schematic diagrams illustrate three cell parameters that are electrically measured using a CMOS IC for live-cell assessment: cell attachment via a cell-substrate impedance, Zₛ; cell-cell adhesion via a transepithelial impedance, Zₜₑ; and the metabolic state via the extracellular redox potential, V_{redox}. Each measurement is non-invasive and fast (e.g., <1 min), allowing the measurements to be repeated sequentially every approximately 5 min to 10 min for real-time investigations. FIG. 23b illustrates a scenario where a fluorescent microscope can be paired with a packaged CMOS IC for simultaneous optical and electrical cell measurements. A reference electrode, Pt (shown) or Ag/AgCl, can be used in this example. FIG. 23c is a colorized fluorescent image of MDCK epithelial cells cultured on top of the electrode array of the CMOS IC. As noted above, the 64×64=4,096 8-μm diameter Pt electrodes, which are circular, are spaced at a 20-μm pitch. PtB can be electrodeposited onto the electrodes to lower the electrode impedance for higher signal-to-noise Zₜₑ measurements. FIG. 23d is a circuit diagram of an example of a pixel circuit for an electrode in the electrode array. As noted herein, each electrode may correspond to a pixel, such that the electrode array may be considered a pixel array, in some embodiments of the present technology. In this example, each of the 4,096 electrodes is connected its own pixel circuit via shielded routing lines, which have dimensions in a range of about 1 mm-10 mm. In this example, the pixel circuit is an op-amp based pixel circuit configured to apply a voltage, Vₛ, and to measure a current via a feedback resistor R_{f} having a value of approximately 100 MΩ, or to apply a current, Iₛ, and to buffer/measure an electrode voltage, Vₑ. An op-amp output, Vₐₘₚ, may be routed off-chip for further processing (e.g., analog-to-digital conversion, etc.). Switches of the pixel circuit may be digitally programmed using a real-time software interface.

In accordance with some aspects of this example, for a high channel count such as 4,096 channels for the 4096 pixels of the pixel array, parallel current and open-circuit potential measurements featured in the measurement techniques in this example may provide unique advantages over other MEA devices. For example, measurements as described in this example may be difficult or even impossible in MEA devices that measure electrode capacitance, or that measure voltage with high-pass filters to block DC signals, or that measure current with a small number of channels (e.g., <32).

### Example 1A: Cell Mapping Using Distribution of Max Current

This example describes a technique for mapping cells using a CMOS-based electrode array that contains a 64×64 array of 4,096 Pt electrodes arranged at a 20-μm pitch.

The inventors have recognized and appreciated that AC cross-electrode impedance measurements between a pair of electrodes can be used to detect cells using a contrast between an insulative cell membrane and a conductive culture medium (e.g., a solution). In a conventional impedance measurement, solution paths around cells may shunt measurements and thus lower detection sensitivity, because a contribution of the measured electrode-to-electrode current from the solution may be far larger than a change in current contribution attributable to the cells. Various embodiments of devices disclosed herein, which may be used for investigations such as the investigation of this example, may improve detection sensitivity by instead measuring a change in electric-field distribution attributable to the cells.

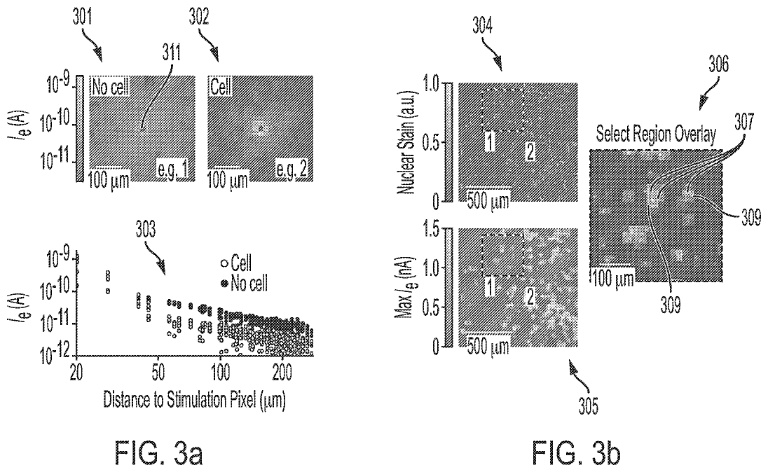

In this investigation, an AC voltage having an amplitude of 200 mV was applied to one electrode and resultant AC currents were measured with respect to the remaining 4,095 electrodes using TIAs. Results are illustrated in FIG. 3a, which shows a measured current-distribution heat map 301 of nearest 11×11 recording electrodes to a single stimulus electrode 311 when no cell is present. In the heat map 301, each pixel corresponds to a location of an electrode. Each electrode has an electrode location or electrode position that may be expressed in a number of ways, such as but not limited to a coordinate or a pixel number. Also shown in FIG. 3a is a measured current-distribution heat map 302, which is similar to the heat map 301 but with a cell on top of the stimulus electrode 311. The cross-electrode impedance measurements correspond to a 1.9 kHz signal frequency.

FIG. 3a also shows a graph 303 plotting measured cross-electrode current versus distance from recording pixel to stimulation (stimulus) pixel, for measurements in the presence of cells (open circles) and with no cell present (filled circles). As evident from the graph 303, the cross-electrode coupling to adjacent electrodes in the presence of cells is higher by almost an order of magnitude in comparison to electrodes without cells on top.

In this example, a fluorescent nuclei MDCK cell line was used for optical confirmation. FIG. 3b shows a fluorescent microscopy image 304 across the entire 64×64 array of electrodes, in which lighter colorations representing fluorescence signals that indicate the presence of cells. To generate a cross-electrode impedance map of the same area as the image 304, the stimulus electrode was sequentially scanned across the array. That is, the electrodes took turns serving as the stimulus electrode. For each given stimulus electrode, cross-electrode current values were measured with other, remaining electrodes serving as recording electrodes. Recorded cross-electrode currents were collected and a maximum value was determined and is referred to as a max current value, Iₑ, corresponding to the given stimulus electrode. Included in FIG. 3b is a heat map 305 across the array of electrode generated, using the max current value, Iₑ, determined for each pixel location serving as the stimulus electrode.

FIG. 3b also shows a select region overlap map 306, which is an overlay of a select region 1 of the fluorescent microscopy image 304 and a select region 1 of the heat map 305. The overlap map 306 show, nuclei fluorescence signals 307 corresponding to the fluorescent microscopy image 304 and shows max current signals 309 corresponding to the heat map 305, and indicates an ability to map a cluster of cells with single-cell resolution. This example demonstrates that the presence of cells can be confirmed using nuclei fluorescence markers with a strong correspondence between the max current map and the fluorescent microscopy image.

The max current value, Iₑ, may be determined for each location of the stimulus electrode using any suitable method based on cross-electrode currents measured from recording electrodes for each location of the stimulus electrode. Such a determination may be comprised of a simple comparison of absolute arithmetic values of the cross-electrode currents, and may additionally include data processing such as noise filtering, and/or background subtraction, and/or any suitable signal processing technique known in the field, which may be performed prior to the comparison. Processing and comparison of the current values may be performed after digitization of measured current values, and may utilize a processing unit such as the processing unit 120 as shown in FIG. 1a.

### Example 2: High Spatial Resolution Mapping Using Cross-Electrode Currents

This example describes a technique for generating an up-scaled map of cross-electrode coupling, which may have a higher spatial resolution than an electrode-to-electrode pitch of an electrode array.

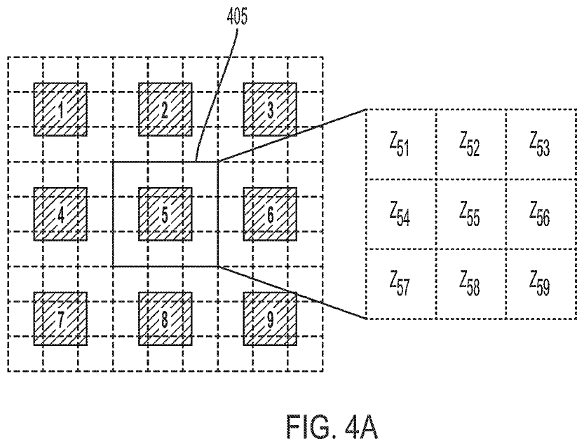

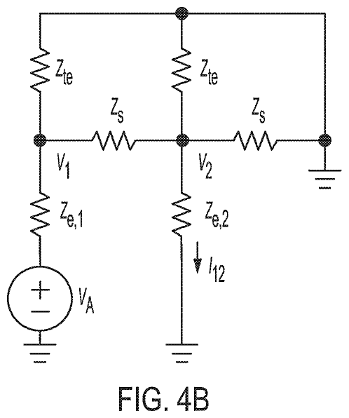

According to some embodiments of the present technology, nearest neighbor cross-electrode measurements may be performed for each electrode of the electrode array serving as a stimulus electrode. FIG. 4A shows an example of a high resolution up-scaled mapping using a 3×3 impedance grid (an example is shown in the inset) for each of nine (9) electrodes 1 through 9. In some embodiments, electrodes located at edges of the electrode array may be skipped from the up-scaled impedance grid as described below.

FIG. 4B is a schematic circuit model that may be used to calculate cell-substrate impedance, Zₛ, and transepithelial impedance, Zₜₑ, for an applied AC stimulation voltage, V_{A}, and to measure a cross electrode current, I₁₂. The 3×3 impedance grid may be used for the Zₛ calculation, and a single Zₜₑ may be extracted for each electrode.

In this technique, to measure cell-substrate attachment, a change in cross-electrode field is formed. During measurements, the electrode array may be exposed to a solution in a culture well. Instead of applying a bias between two electrodes, a bias is applied from one electrode, serving as a stimulus electrode, to all remaining electrodes, serving as recording electrodes. This allows field lines starting from the stimulus electrode to extend far up into the solution in the culture well and return to terminate on electrodes far away from the stimulus electrode. Otherwise, the field lines would need to curl back towards adjacent electrodes, increasing an amount of measured current that is not related to an immediate cell-electrode interface of interest.

The interface may be modeled using a cross-sectional type of model to increase spatial resolution. If it is assumed that Zₛ<<Zₜₑ, Z_{e,1}, and Z_{e,2}, which according to some aspects are found to be valid for most measurements, then:

$${V}_{2} \approx \frac{1}{2} {V}_{1} \approx \frac{1}{2} {V}_{A} \frac{2 {Z}_{s}}{{Z}_{1} + 2 {Z}_{s}} \approx {V}_{A} \frac{{Z}_{s}}{{Z}_{1}} . \qquad ( Eq . A 1 )$$

A measured cross-electrode current can also be written and expressed in terms of Eq. A1, as follows:

$${I}_{12} = \frac{{V}_{2}}{{Z}_{2}} \approx {V}_{A} \frac{{Z}_{s}}{{Z}_{1} {Z}_{2}} . \qquad ( Eq . A 2 )$$

To determine Z_{e,1} and Z_{e,2}, a sum of measured currents across the electrode array is used when a stimulus signal is applied to an electrode n, as follows:

$${Z}_{e , n} = \frac{{V}_{A}}{{I}_{n}} . \qquad ( Eq . A3 )$$

Zₛ can then be calculated using Eq. A3 and Eq. A2, as follows:

$${Z}_{s} = {V}_{A} \frac{{I}_{12}}{{I}_{1} {I}_{2}} , \qquad ( Eq . A 4 )$$

which uses all measured currents.

To generate a high-spatial-resolution map of the cell-substrate impedance, Zₛ, nearest neighbor cross-electrode measurements may be used for each stimulus electrode, which includes using a 3×3 grid for each electrode serving as the stimulus electrode (except electrodes at edges of the electrode array), as discussed above in connection with FIG. 4A. This creates an overall Zₛ image of 190×190 pixels (in comparison to the 64×64 electrodes in the electrode array). Note that the word “pixel” when used in connection with a 3×3 grid for an electrode is not the same as when used in connection with a pixel array, which sometimes may be used herein to refer to an electrode array.

In the inset shown in FIG. 4A for a center electrode 5, each of the nine pixels in the 3×3 grid 405 for the center electrode 5 is filled in using normalized impedance values Z based on measured currents to nearest neighboring electrodes. Each normalized impedance value Z may be calculated as follows:

$${Z}_{52} = {V}_{AC} \frac{{I}_{52}}{{I}_{5} {I}_{2}} , {Z}_{54} = {V}_{AC} \frac{{I}_{54}}{{I}_{5} {I}_{4}} , {Z}_{56} = {V}_{AC} \frac{{I}_{56}}{{I}_{5} {I}_{6}} , {Z}_{58} = {V}_{AC} \frac{{I}_{58}}{{I}_{5} {I}_{8}} , \qquad ( Eq . 1 )$$

where V_{AC} is an amplitude of an applied AC voltage, I_{xy} is a magnitude of an AC current measured for electrode y when the AC signal is applied to electrode x, and Iₓ [I_{y}] is a sum of the magnitude of the AC currents measured by all other electrodes when the AC signal is applied to electrode x [y]. The edge normalized impedance values may then be calculated as follows:

$${Z}_{51} = \frac{{V}_{AC}}{\sqrt{2}} \frac{{I}_{51}}{{I}_{5} {I}_{1}} , {Z}_{53} = \frac{{V}_{AC}}{\sqrt{2}} \frac{{I}_{53}}{{I}_{5} {I}_{3}} , {Z}_{57} = \frac{{V}_{AC}}{\sqrt{2}} \frac{{I}_{57}}{{I}_{5} {I}_{7}} , {Z}_{59} = \frac{{V}_{AC}}{\sqrt{2}} \frac{{I}_{59}}{{I}_{5} {I}_{9}} , \qquad ( Eq . 2 )$$

where the square root of 2 was determined to normalize the difference in distance between the edge and corner electrodes. The center normalized impedance value may then be determined as follows:

$${Z}_{55} = median ( {Z}_{52} , {Z}_{54} , {Z}_{56} , {Z}_{58} ) . \qquad ( Eq . 3 )$$

The use of the cross-electrode currents not only increases an effective spatial resolution in comparison to using a max of the current distribution but it may also allow for unadhered cells, which may cause a decrease in a cross-electrode current, to be mapped.

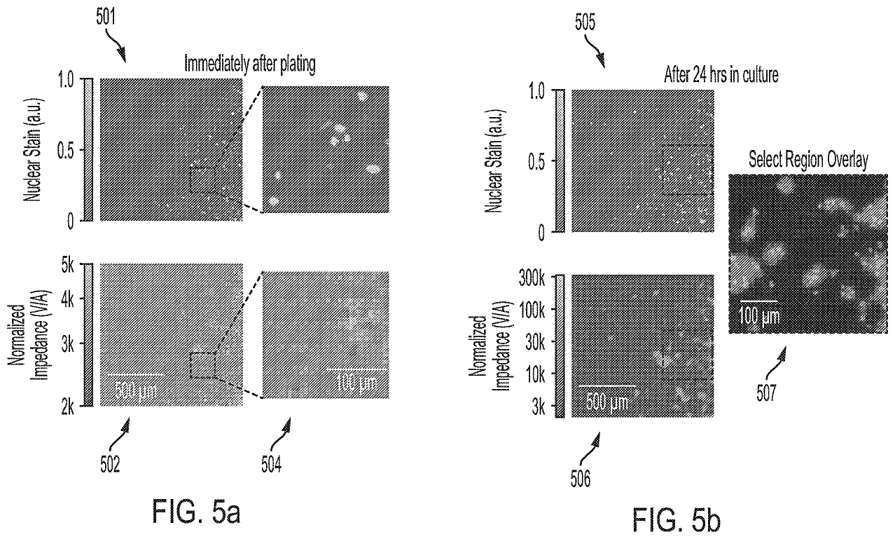

FIGS. 5a and 5b illustrate an example of up-scaled cross-electrode impedance mapping in comparison with a fluorescent microscopy imaging, according to some embodiments of the present technology. FIG. 5a shows a fluorescent microscopy image 501 across an electrode array, and a heat map 502 of a normalized cross-electrode impedance of a cell culture immediately following plating. Included in FIG. 5a is an inset showing an enlarged portion of the fluorescent microscopy image 501 and an inset showing an enlarged portion 504 of the heat map 502, revealing a decrease in the cross-electrode normalized cell-substrate impedance, Zₛ, for unadhered cells with single-cell resolution. Stated differently, electrodes that were covered by cells showed a higher impedance than electrodes that were not covered by cells. The heat map 502, which is a mapping immediately following a plating of cells such that the cells are not adhered, shows smaller normalized impedance values when electrodes are covered with cells in comparison to non-covered electrodes.

FIG. 5b shows a fluorescent microscopy image 505 and a cross-electrode impedance map 506 after 24 hours of exposure to a culture. FIG. 5b also shows an overlay map 507 corresponding to an enlargement of a select region of the fluorescent microscopy image 505 and the cross-electrode impedance map 506. The results show that many cells have adhered to the surfaces of the electrodes, causing a drastic increase in normalized cross-electrode impedance values.

### Example 3: Quantifying Cell Adhesion

This example describes a technique that uses cross-electrode impedance mapping to quantify adhesion or adherence of cells in a cell culture. The terms “adhesion” and “adherence” may be used interchangeably herein.

In this example, ethylenediaminetetraacetic acid (EDTA) was applied to the cells. EDTA is a calcium chelator that removes Ca²⁺ needed for integrin proteins to maintain cell adhesion. With EDTA applied, the cells quickly detached over a duration of time of about 50 min. The EDTA was then washed out by adding normal culture media, where the cells re-attached over a duration of time of about 200 min.

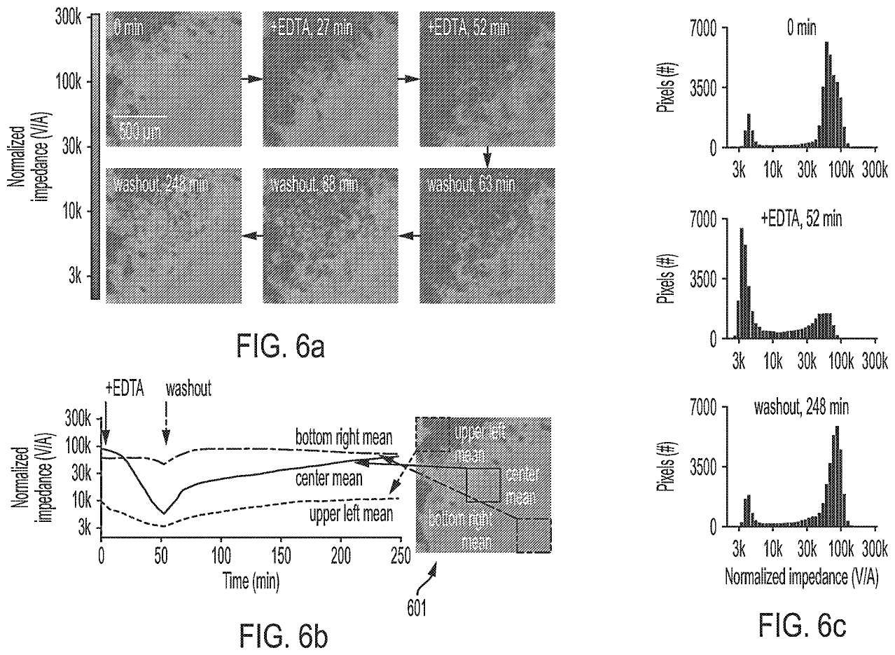

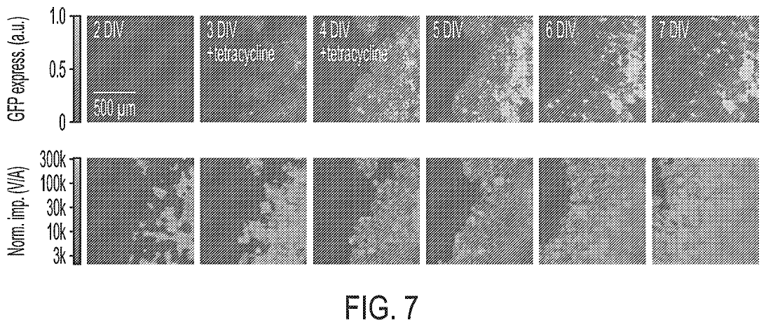

The detachment and reattachment of the cells was captured with high spatial and time resolution by cross-electrode impedance mapping, as depicted in FIG. 6a, which shows a series of normalized impedance maps over time for MDCK cells with a 5 mM EDTA application and a subsequent washout.

FIG. 6b is a data plot showing mean normalized impedance values over time for different regions of a map 601 of an electrode array in the cell culture. FIG. 6c show histograms of the normalized impedance values before, during, and after a washout of EDTA across the electrode array.

To show a biologically relevant example of quantification of cell adhesion, a genetically modified MDCK cell line was measured wherein tetracycline was used to turn on and off a RasV12 and GFP gene. FIG. 7 shows results comprised of a series of fluorescent microscopy images and normalized cross-electrode impedance maps of MDCK cells over seven (7) days of culture in vitro (DIV). Tetracycline was added after the 2 DIV measurement to turn on the gene RasV12, which is related to cancer. This gene also expresses GFP such that the gene expression can be imaged. The tetracycline was then removed after the 4 DIV measurement to turn off the gene expression. The cells were found to be less adherent to the surfaces of the electrodes when the RasV12 gene was expressed and were found to return to normal after gene expression was turned off.

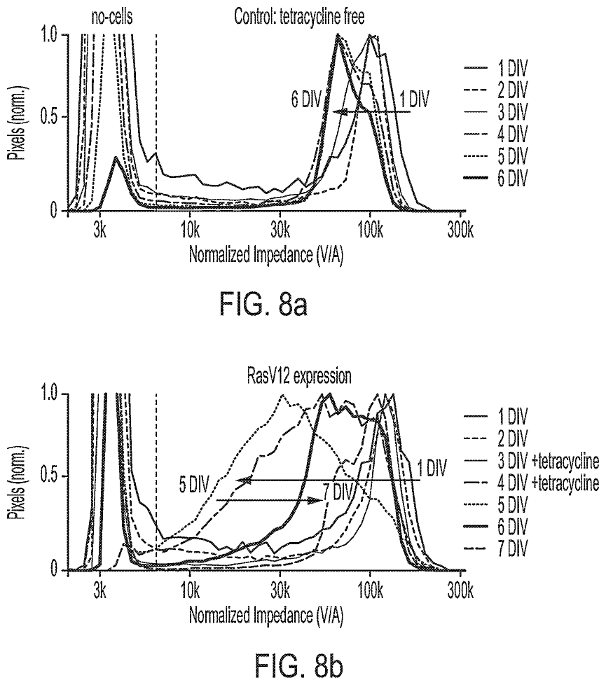

RasV12 is an oncogene and has been known to increase cell metabolism and decrease cell adhesion when strongly expressed, which together can cause cancer-like cell growth and tumors. Initially, tetracycline was kept out of the culture media and the cells were adhered as normal. When tetracycline was introduced, the genes were expressed causing an increase in GFP and a decrease in cell adhesion. Removal of tetracycline then reversed the cell adhesion to cause the cells to more strongly adhere while also decreasing overall GFP expression; however, some portions of the cell culture did not turn off as strongly as others. The effects on cell adhesion were quantitatively compared to a control culture which did not have tetracycline introduced, as shown in FIGS. 8a and 8b. FIG. 8b is a normalized impedance histogram of MDCK cells over a span of 6 to 7 days of culture in vitro (DIV). Tetracycline was added after the 2 DIV measurement to turn on the gene RasV12, which is related to cancer. FIG. 8a is a normalized impedance histogram of a control measurement without the addition of tetracycline. The histograms were normalized to the max pixel number above the no-cell impedance values of about 8 kΩ. The cell adhesion was reduced in comparison to the control, which showed a smaller decreasing trend over time.

### Example 4: Frequency Response

This example demonstrates how frequency may have an effect in the cross-electrode impedance measurements.

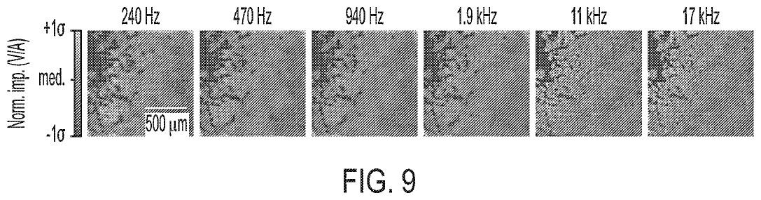

In this example, frequency of mapping was swept to determine a best frequency for measuring cell adhesion using cross-electrode impedance mapping. FIG. 9 shows a series of normalized cross-electrode impedance maps under stimulus signals of different frequencies. The plots are normalized to a median+/−1 standard deviation. The lower frequencies show higher signal contrast, which correlates to the optically measured GFP fluorescence data shown in FIG. 7. This indicates that a lower frequency is better for measuring cell adhesion. As can be seen from FIG. 9, the frequency of 1.9 kHz used for some studies shows a relatively good contrast in comparison with the frequency of 240 Hz. However, above 10 kHz, the maps look relatively uniform.

### Example 5: Cell-to-Cell Adhesion

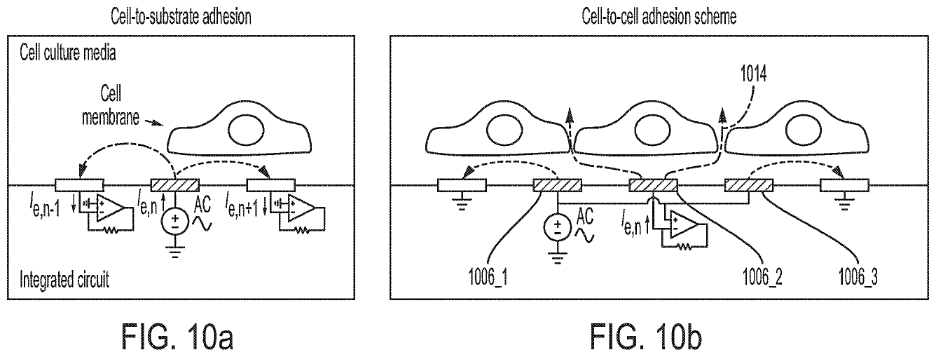

The previous examples relate to how to map cells and their adhesion over time via cross-electrode impedance measurements, such as schematically depicted in FIG. 10a, for example. In FIG. 10a, an AC voltage may be applied to a single electrode of an electrode array and currents may be measured through the electrode array's other electrodes using, e.g., TIAs. The adhesion or adherence is mainly a function of the cell-to-substrate attachment and resultant height of a cell-to-substrate gap.

This example describes a technique that may be used to measure cell-to-cell attachment, or how well connected the cells are to each other. Cells in a culture not only may attach to surfaces, but also to each other via cell-cell connections. The tightness or strength of these connections may be used to define a permeability of a sheet of cells, which may be important for epithelial tissues that can act as barriers of body surfaces, internal organ linings, and other tissues. In this example, a barrier function relating to this barrier may be measured by performing a map of transepithelial impedance, Zₜₑ. In this way, cell-cell connectivity, au be assessed using electrodes only covered by cells, to mitigate any holes while also allowing for assessment of spatial heterogeneity.

In this example, a stimulation protocol may be modified to measure the vertical field component 1014, as shown in FIG. 10b, which schematically depicts an arrangement for a measurement step according to some embodiments of the technology disclosed herein. In FIG. 10b, a center electrode 1006_2 and its surrounding electrodes 1006_1, 1006_3 may be biased with an AC voltage. The center electrode 1006_2 and its surrounding electrodes 1006_1, 1006_3 may be part of an array of electrodes. A current, I_{e,n}, may be measured through the center electrode 1006_2. Because the center electrode 1006_2 and its surrounding electrodes 1006_1, 1006_3 are biased with the same AC voltage, the center electrode 1006_2 will not pass current to its surrounding electrodes 1006_1, 1006_3, and will only pass current due to an impedance of a sheet of cells above the center electrode 1006_2. Outside of the center electrode 1006_2 and its surrounding electrodes 1006_1, 1006_3, a remainder of the array of electrodes may be biased to ground or to a reference voltage level to act as a current return. This type of measurement is similar to measurement of transepithelial electrical resistance (TEER), which typically may be measured using an arrangement comprised of two electrodes on opposite sides of a cell culture on a suspended porous membrane. For example, a device having the arrangement shown in FIG. 10b may be used to map TEER across the cells on top of an electrode array without the need for special suspension. In some embodiments, advantages of such an arrangement for TEER measurements may include: a need for fewer cells, an ability to assess spatial heterogeneity, and an ability to combine cell-to-cell and cell-to-substrate adhesion measurements using a same device.

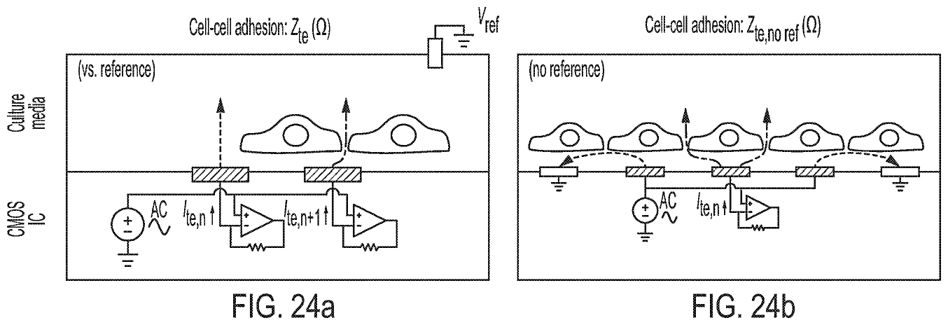

FIGS. 24a and 24b are schematic diagrams illustrating some additional schemes that may be used for cell-cell connectivity measurements, in accordance with some embodiments of the present technology. A change in vertical field above an electrode may be measured to isolate effects of cell-cell connections using two circuit configurations: 1) a fast (e.g., less than about 1 s/measurement) parallel electrode measurement versus a reference, using the scheme illustrated in FIGS. 24a, and 2) a slow scanned (e.g., greater than or equal to about 40 s/measurement) relative measurement without a reference using the scheme illustrated in FIG. 24b. The fast measurement may be well suited for sweeps across multiple frequencies whereas the slow scanned measurement, which may not require a reference, may be advantageous for making long-term measurements more stable and may be well suited for device miniaturization. For both types of measurements, an optional PtB layer may be used to lower Zₑ by about a factor of five (5) to improve Zₜₑ sensitivity. Experiments across frequencies show that mid-range frequencies of approximately 2 kHz to 5 kHz were best for assessing cell-cell connectivity.

Calculation of transepithelial impedance, Zₜₑ, will now be discussed in connection with the schemes illustrated in FIGS. 24a and 24b.

To measure cell-to-cell attachment, or how well connected the cells are to each other, a stimulation protocol used to measure an electric field's vertical field component in the schemes illustrated in FIGS. 24a and 24b, may be modified. Measurements can be made relative to a reference potential, Vref, (see FIG. 24a) by applying an AC voltage to all electrodes to measure each transepithelial electrode current, I_{te,n} (n=1, 2, . . . , 4096), which may be measured via TIAs at a measurement duration of, e.g., 1 s/frequency. The reference potential, Vref, may be a ground potential. The resultant field distribution may be vertically correlated with cell connectivity decreasing the transepithelial electrode current, Iₜₑ. Non-reference measurements can be made by applying an AC voltage to an electrode (n) and its neighboring electrodes to create an effective vertical field measurement, with remaining (non-neighboring) electrodes being grounded (see FIG. 24b). To generate a cell map, the applied voltage may be scanned across the array at a measurement duration of, e.g., 40 s per scan/frequency.

In the parallel scheme illustrated in FIG. 24a, an AC voltage may be applied to each electrode of an array of electrodes relative to a reference voltage, Vref. Each electrode's current, I_{te,n}, which may be a current associated with a vertical component of the electric field, may be measured in parallel with one or more other electrodes of the array. The array may be in an environment comprised of a solution containing cells being studied. With such a scheme, a vertical field may be established in the solution. Electrodes at the array's periphery may also have a fringing field at low frequencies. Because a current at an electrode may need to go through a cell sheet, a magnitude of the current at the electrode may be proportional to the electrode's transepithelial impedance, Zₜₑ.

In the scanned scheme illustrated in FIG. 24b, a center electrode and its surrounding electrodes may be biased with an AC voltage, and current may be measure only through the center electrode. The center electrode may not pass current to surrounding electrodes because they are biased with the same AC voltage; therefore, the center electrode may only pass current due to an impedance of the cell sheet above the electrode. Outside of the center electrode and its surrounding electrodes, the remainder of the array may be biased to ground potential to act as a current return.

In either the parallel scheme illustrated in FIG. 24a or the scanned scheme illustrated in FIG. 24b, the measured vertical current I_{te,n} may be expressed as follows:

$${I}_{te , n} = \frac{{V}_{A}}{{Z}_{e , n} + {Z}_{te}} . \qquad ( Eq . A8 )$$

Using Eq. A3, Zₜₑ can be solved using the following expression:

$${Z}_{te} = \frac{{V}_{A}}{{I}_{te , n}} - \frac{{V}_{A}}{{I}_{n}} . \qquad ( Eq . A9 )$$

For measurements, it was determined that mid-frequencies in a range of about 1 kHz to 5 kHz are best correlated with cell-cell connectivity (see also Example 15, below). For the PtB electrodes, Z_{e,n} may then be sufficiently smaller than Zₜₑ (see also Example 15, below) such that Eq. 8 may be used to arrive at the following simplified expression:

$${Z}_{te} = \frac{{V}_{A}}{{I}_{te , n}} . \qquad ( Eq . A10 )$$

For Zₜₑ experiments with just Pt electrodes, an Iₙ measurement from the cell-substrate impedance may be such that the right-hand-side portion of Eq. 9 may be disregarded. For scanned array measurements (see FIG. 24b) to calculate Z_{te,no ref} using a 3×3 set of electrodes, a total map may be generated that is comprised of 62×62 pixels, because peripheral electrodes do not have neighboring biased electrodes to create a vertical field. For parallel array measurements (see FIG. 24a) that use a reference voltage, a map containing 64×64 pixels may be generated.

### Example 5A: Metabolic State Mapping Via Extracellular Redox Potential, V_{redox}

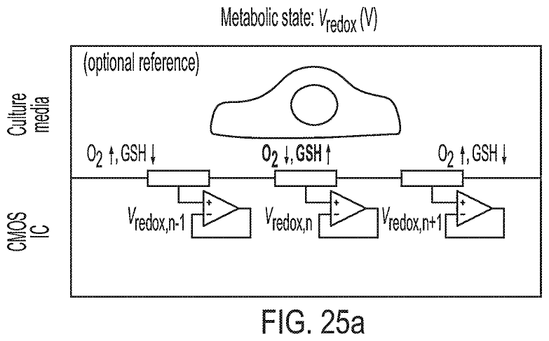

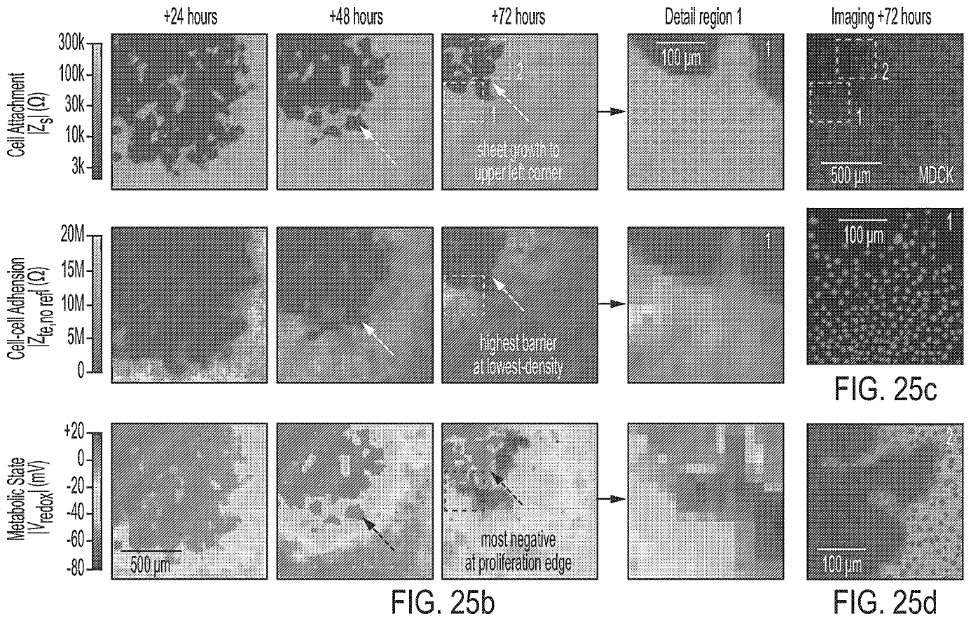

In addition to impedance measurements, platinum (Pt) electrodes have been used for both potentiometric sensing of oxygen and extracellular redox monitoring. This example demonstrates that a proximate location of Pt electrodes directly underneath live cells may be used to map the extracellular redox potential, V_{redox}, in situ to monitor a redox environment of cells and even to monitor O₂ consumption to map a metabolic state of cell cultures. To accomplish such measurements, a pixel amplifier may be configured as a buffer, as schematically shown in FIG. 25a.

In general, cells may use energy arising from movement of electrons from oxidizable organic molecules (e.g., glucose) to O₂ during aerobic metabolism. To help mediate such electron flows, a general reducing environment may be created by the thiol-compound glutathione (GSH), which is often considered to be a cellular redox buffer. In simplified terms, a cell's redox potential may then be a balance between O₂ increasing the potential (oxidation) and GSH reducing the potential (reduction). The redox environment is important not only for electron transfer but also for neutralizing harmful reactive oxygen species, cell-cell signaling, and regulating the state of the cell. For example, ranging from negative to positive, the redox potential may determine whether a cell is in a state of proliferation, differentiation, apoptosis, or necrosis.

Results of multi-parametric measurements are shown in a series of data maps in FIG. 25b. The measurements, which were performed at +24, +48, and +72 hours after MDCK cell plating, include cell attachment (top row), cell-cell adhesion (middle row), and metabolic state (bottom row). In each map, the cells were observed to exhibit growth from the bottom right to the upper left corner, where the proliferating leading edge cells show the most negative V_{redox} in comparison to the more dormant trailing edge cells. The Zₜₑ is highest at the leading edge as well, due to the lowest density of cells (see magnifications in Detail region 1 column), and therefore exhibits the fewest cell-cell connections. FIG. 25c shows a pair of nuclei fluorescence images at +72 hours: after plating (top image) and a magnification of region 1 for comparison (bottom image), indicating the lowest cell density on the leading edge in comparison to the trailing edge. FIG. 25d is a composite map of a magnification of region 2 in which images for cell nuclei and cell attachment are overlaid, indicating good spatial correspondence with single-cell resolution.

One goal of this example is to investigate what information the proximate V_{redox} could provide by pairing it with the impedance techniques to monitor cell growth (FIG. 25b). In this example, a negative V_{redox} in the range of 30 mV to 80 mV was observed for electrodes with cells in comparison to electrodes without cells (FIG. 25b). From a comparison of details of the regions in the magnified images, the spatial information of V_{redox} is distinct and different from the cell attachment or cell barrier, where the most negative V_{redox} is at the leading edge and not the lowest density. In general terms, the negative signal could indicate a locally smaller [O₂] or locally higher [GSH] near the cells.

To further explore the V_{redox} signal's origin, O₂ dependence was tested via an oxygen purge on a separate MDCK cell culture. Upon O₂ removal, a signal difference between regions with cells and without was eliminated. To complement, the GSH based reducing capacity was tested via an oxidative titration. Ferricyanide, [Fe(CN)6]³⁻, was chosen for the titration due to its previous non-toxic use in cell-cultures and its oxidizing half-cell potential in comparison to the cellular environment. The media showed a 4 μM reducing capacity while the cells had a much larger capacity of greater than 200 μM.

Taken together, these measurements show that the measured V_{redox} may be related to both the in situ [O₂] and [GSH]-based reducing capacity of the cells. The inventors theorize that with aerobic respiration, the [O₂] is reduced from its normal dissolved concentration of ˜200 μM at atmospheric conditions, which lowers V_{redox} until it is regulated by the extracellular reducing potential of the cells. Therefore, though it may be difficult to quantify an oxygen consumption rate with this technique, the V_{redox} measurement of the extracellular redox potential may be useful for monitoring the metabolic state of cells, as it can show both the usage of O₂ and the reducing environment of cells. Therefore, the more negative signal on the leading edge of the cell sheet (FIG. 25b) may be attributed to respiration combined with a state of proliferation, the most negative redox potential state of a cell.

### Example 6: Antibody-Cell Binding

Screening for antibody-cell binding can be a low-throughput process due the need for either fluorescent tagging of the antibodies, which requires wash steps to remove non-bound fluorescent antibodies, or the need for a special optical measurement such as surface plasmon resonance (SPR). According to some embodiments of the present technology, the cross-electrode impedance techniques described herein may offer an ability to measure an antibody-cell binding event through either cell-to-substrate measurements or cell-to-cell adhesion measurements. With the binding of an antibody on an underside of a cell, the gap distance becomes effectively smaller leading to a decrease in the amount of cross-electrode current measured. Likewise, with antibody binding to sides of the cells, the cell-to-cell gap distance should also become smaller, which may lead to a decrease in the amount of vertical current measured. Being able to perform such antibody binding without labels then allows for different antibodies to be added in sequence without the need for wash steps, which may greatly improve throughput.

### Example 7: Cell Patterning Through Electrochemical Gas Generation

This example describes a method for patterning cells on top of an electrode array. The inventors have recognized and appreciated that small gas bubbles can be electrochemically generated to produce small holes in the cell membrane to kill the cells via depolarization. After death, the dead cells will then detach from the surface, as schematically illustrated in FIG. 11. Therefore, by controlling which electrodes generate gas, the cells can be patterned with the spatial resolution of the electrode array.

Without wishing to be bound to a particular theory, the inventors recognized that for most inert electrode materials (e.g., platinum, gold, etc.) hydrogen gas can be generated by adjusting the electrode potential below the hydrogen ion/hydrogen gas redox half-cell reduction potential (E⁰), according to the following relationship:

$$\begin{matrix}2 {H}^{+} + 2 {e}^{-} ⇌ {H}_{2} ( g ) & {E}^{0} = 0. V .\end{matrix} \qquad ( Eq . 4 )$$

or oxygen gas can be generated by adjusting the electrode potential above the oxygen gas/water redox potential, according to the following relationship:

$$\begin{matrix}{O}_{2} ( g ) + 4 {H}^{+} + 4 {e}^{-} ⇌ 2 {H}_{2} O & {E}^{0} = + 1.23 V .\end{matrix} \qquad ( Eq . 5 )$$

Likewise, as most cell media contain chloride salts, chloride gas can be generated by adjusting the electrode potential above the chlorine gas/chloride redox potential, according to the following relationship:

$$\begin{matrix}{Cl}_{2} ( g ) + 2 {e}^{-} ⇌ 2 {Cl}^{-} & {E}^{0} = + 1.36 V .\end{matrix} \qquad ( Eq . 6 )$$

Accordingly, cell removal may be performed by selectively applying a pre-determined potential that is above a redox potential for generation of a gas at one or more electrode locations. The potential may be applied, e.g., by connecting one or more stimulus circuits 110 (see FIG. 1a) to selected electrodes. The potential does not need to be identical across all the selected electrodes, and programmable heterogeneity may be used when electrodes are biased differently. The potential may be a potential relative to a potential of a reference electrode in the medium above the electrodes.

For more controllable patterning, an electrode current can be used to set the electron transfer rate and therefore the rate of gas generation. Controlling the rate of gas generation may optimize the selective electrochemical reaction as large bubbles can form on the surface by using a gas generation rate that is too fast, blocking the electrodes from the solution or medium.

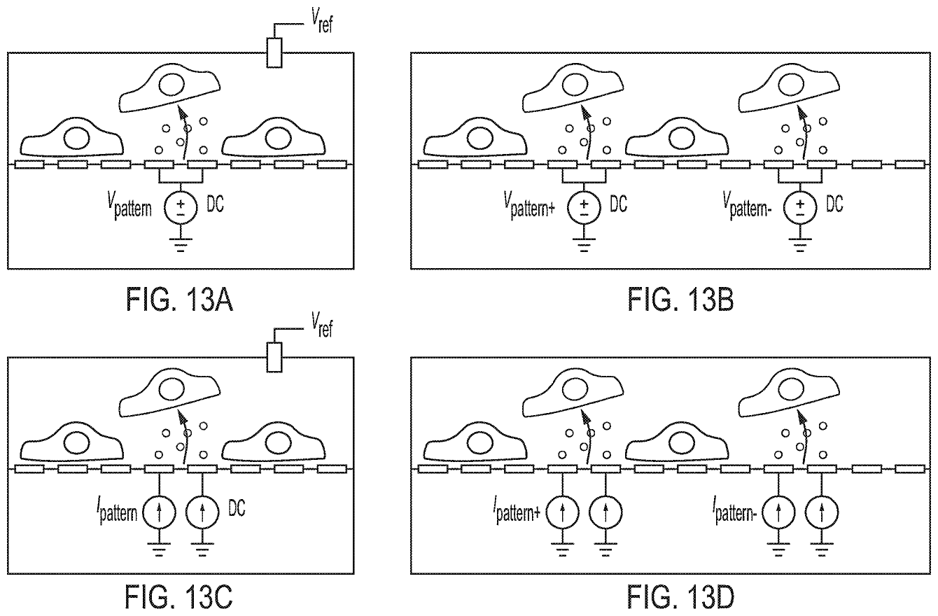

FIGS. 13a through 13d show a series of schematic diagrams illustrating arrangements for cell patterning using an electrode array. FIGS. 13a and 13b illustrate diagrams for arrangements where one or more pre-determined patterning voltages are applied to selected electrodes for patterned removal of a cell by electrochemical gas generation. FIGS. 13c and 13d illustrate diagrams for arrangements where one or more pre-determined patterning currents are applied to selected electrodes for patterned removal of a cell. FIGS. 13a and 13c illustrate an example of voltage/current patterning with a reference electrode acting as a return. FIGS. 13b and 13d illustrate an example of differential voltage/current patterning using cross-electrode gas generation without using a reference electrode, where one set of electrodes passes a positive current and a second set of electrodes passes a negative current (return).

### Example 8: Cell Spatial Patterning and Defining a Co-Culture

This example describes spatial patterning of cells and definition of a co-culture using an electrode array.

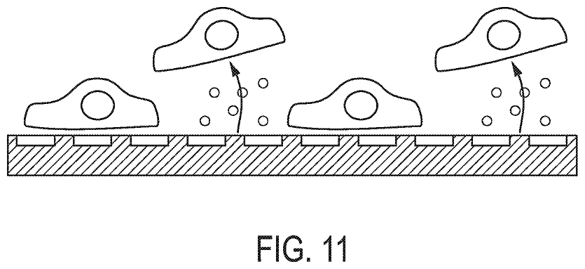

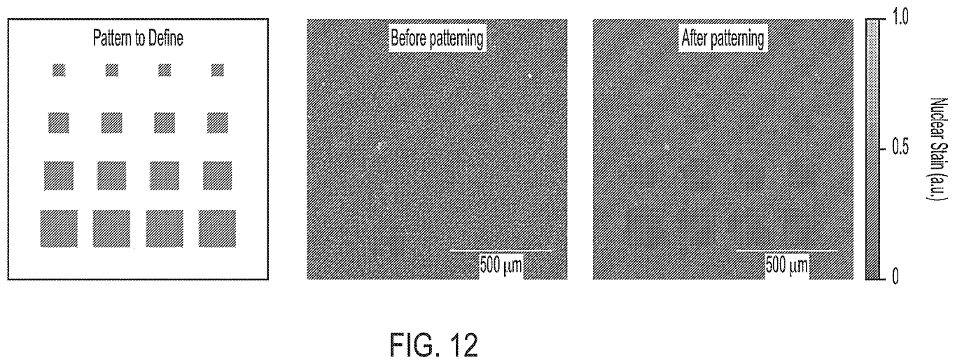

FIG. 12 shows a CMOS electrode array that was used with MDCK cells. The left panel shows the electrode pattern or layout of the electrode array; the middle panel shows an image of the electrode array before patterning; and the right panel shows an image of the electrode array after patterning. In this example, H₂ gas was generated by applying −1.25 V to the platinum electrodes versus a Ag/AgCl pseudo reference electrode. FIG. 12 shows fluorescent microscope images of the electrodes before (middle) and after (right) applying a patterning voltage for 80 seconds. The images show that a cell pattern defined successfully based on the electrode pattern of the electrode array. With an electrode pitch of 20 μm, square holes of various sizes were made in the uniform cell sheet with high spatial resolution, as confirmed using nuclei fluorescent markers and fluorescent imaging.

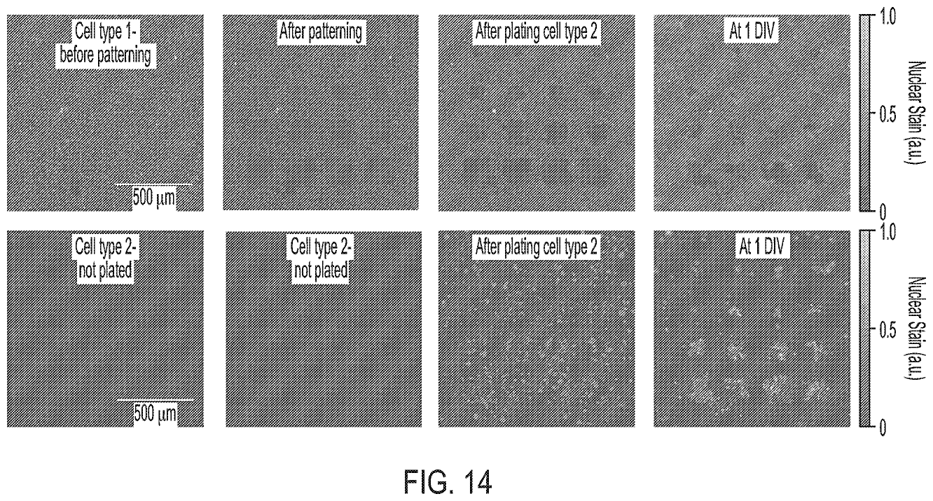

FIG. 14 shows a series of fluorescent microscope images illustrating the process of defining a co-culture via patterning and then plating a second cell type. The cell types were distinguished by different nuclei fluorescent markers. In the experiment resulting in the images in FIG. 14, a co-culture of two different cell types was defined by plating a second MDCK cell line with a different nuclei fluorescent marker after the initial patterning. The second cell type filled in the generated space, showing the ability to spatially define co-cultures with high spatial resolution. Further patterning and plating could also be performed to define multiple cell co-cultures and patterns in a bottom-up approach.

### Example 9: Directed Cell Evolution by Removing Culture Heterogeneity

This example describes a method of directed cell evolution to eliminate from the cell culture cells whose properties are not desired.

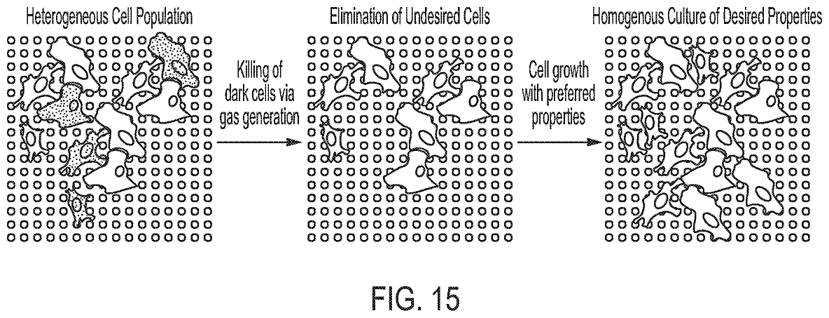

FIG. 15 shows a series of schematic diagrams illustrating a heterogeneous cell population; elimination of undesired cells using patterned electrochemical gas generation on select electrodes; and a homogenous culture of desired properties after subsequent cell growth. The choice of which cells to eliminate can be made via optical imaging or via other properties measured using the electrode array. The capability to eliminate cells from the culture without having to remove the culture plate is advantageous over current processes which would require suspending the cells and separating using a cell sorting machine with a further replating step to again culture, or removal of single cells with the desired properties using a micropipette and then replating. Furthermore, the lineage of the cell history can be preserved as the spatial location of each cell does not change as the cells remain adhered during the process. Such an elimination process could also be used for further analyses to be performed to on a subset of cells after culturing the electrode array, where cells unwanted for further measurement are first killed before cell suspension and removal.

### Example 10: Wound Healing Assay

This example describes a combined application of cross-electrode impedance mapping and cell patterning for a wound healing assay. Such assays may be used to gauge cell growth rate and metabolism and may be useful for screening drugs affecting these parameters. Compared to the electrochemical patterning described herein, other tools mechanically generate a wound in a cell culture via a mechanical scratch which is both difficult to control and limiting in terms of wound pattern.

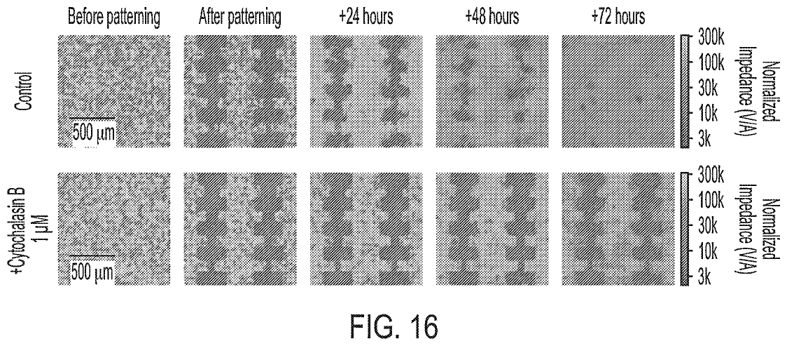

In this example, a wound is made in MDCK cells and then growth is mapped in real time. FIG. 16 shows a sawtooth-like pattern in the cells defined in the center of the device surface with varying distances of separation. These patterns were defined by applying electrode currents of −10 nA for 40 s versus a Ag/AgCl pseudo reference electrode. The regrowth of the culture was then measured using the impedance mapping method. A typical cell culture took approximately 3 days to fill in the wound whereas a culture with a growth inhibition drug showed very little regrowth. As illustrated by the normalized cross-electrode impedance maps in FIG. 16, the control culture shows regrowth after 72 hours in culture. A second culture with a drug that slows growth, cytochalasin B (1 μM), shows very little growth over the course of 72 hours, demonstrating the ability of the assay for drug screening.

### Example 11: Molecular Delivery

This example describes a technique using planar electrodes for membrane permeabilization and molecular delivery. Unlike electroporation, which applies a concentrated electric field to break down the cell membrane (e.g., see FIG. 34), planar electrode permeabilization may be performed via gas bubble formation, similar in concept to the patterning techniques discussed herein. Unlike patterning cells, where cells are killed to perform the patterning, for molecular delivery smaller holes may be created on the cells that will then reseal over time.

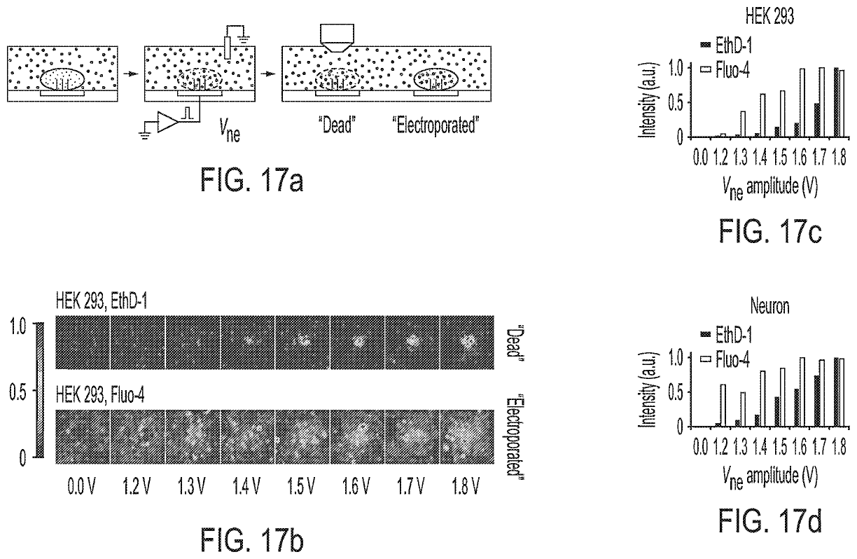

FIGS. 17a through 17d (collectively “FIG. 17”) illustrate an experiment demonstrating electroporation or permeabilization techniques using nanowire electrodes.

Aspects of such techniques may be used with an electrode array comprised of planar electrodes. In a permeabilization experiment schematically shown in FIG. 17a, Fluo-4, a live assay, may be dissolved in an extracellular solution (left panel, FIG. 17a). Electroporation protocols may be applied to nanoelectrodes using a pixel stimulator (middle panel, FIG. 17a) and allowed to recover in the Fluo-4. If successfully electroporated, Fluo-4 may permeate into the cell. After recovery, a dead assay, EthD-1, may be dissolved in the extracellular solution to reveal whether cells died due to irreversible electroporation (right panel, FIG. 17a). Cells that are successfully electroporated and recover may retain Fluo-4 for imaging. FIG. 17b shows heat maps for experimental results, showing EthD-1 and Fluo-4 intensity averaged across eight investigated protocols of increasing voltage amplitude (3 trains of 5 biphasic pulses at 20 Hz) performed with HEK 293 cells. A CMOS nanoelectrode array (CNEA) was divided into subgroups of 128 pixels for each of the eight protocols and repeated in a grid across the array. Imaging was performed on each pixel and the 128 images for each protocol were averaged together. FIG. 17c shows the averaged intensity results from FIG. 17b for the HEK 293 cells. Successful electroporation was observed viewed starting at about 1.3 V, whereas irreversible electroporation was observed starting at about ˜1.7 V. FIG. 17d shows averaged intensity results for neurons for the same test conditions as for the HEK 293 cells, indicating a lower threshold for successful electroporation, less than 1.2 V, and irreversible electroporation at about 1.5 V.

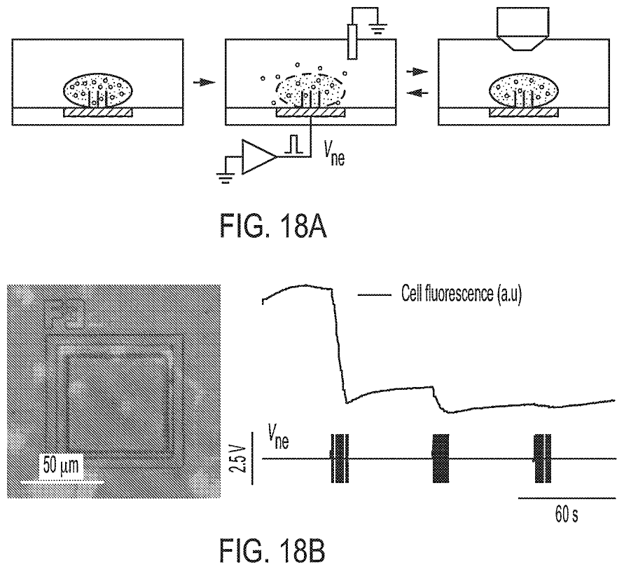

FIGS. 18A and 18B (collectively “FIG. 18”) illustrate another experiment demonstrating electroporation or permeabilization techniques using nanoelectrodes, in which Fluo-4 is injected into cells using Fluo-4 AM. In an electroporation experiment schematically shown in FIG. 18A, electroporation protocols may be applied to nanoelectrodes using a pixel stimulator (middle panel) while fluorescence is monitored over time. If a cell is successfully electroporated, Fluo-4 may be able to flow out of the cell causing a decrease in fluorescence. For successful protocols, the cell's membrane may recover after electroporation (right panel). FIG. 18B shows an image of a neuron in fluorescence from an applied electroporation signal, and also shows a chart of fluorescence over time. As indicated in the chart, the fluorescence drops during electroporation then, immediately afterwards, the cell's membrane recovers and causes the fluorescence to plateau. This indicates that electroporation signals may be applied multiple times without affecting cell viability.

In the experiments of FIG. 17 and FIG. 18, it was observed that voltage signals needed to be of a certain duration, at least 50 ms, to produce observable permeabilization or delivery. This may point to a need for Faradaic processes to generate gas bubbles, as the voltage needed also may be comparable to water window voltages (H₂ & O₂ gas generation via water splitting) when platinum electrodes are used. In FIG. 18, such permeabilization signals were shown to be effective by causing transient leakage of a fluorescent dye, while in FIG. 17, a fluorescent dye was delivered to the cells.

The delivery capabilities demonstrated by the experiments of FIG. 17 and FIG. 18 may be readily used for screening membrane impermeable compounds for their effects on cells and cell-to-cell interactions. The spatial capabilities of the electrode array, in which cells can be chosen for delivery, may be useful in this latter application of cell-to-cell interactions where the delivered cell and its undelivered neighbors can be measured for the effects of the compounds. Without such delivery capabilities, membrane-impermeable compounds would need to be chemically modified for delivery, which is expensive and time-consuming, or would need to be delivered using a micropipette on a single-cell basis, which is also expensive and time-consuming. Beyond compounds, RNA/DNA/plasmids also may be delivered for applications to synthetic biology.

### Example 12: Serial Delivery for Cross-Effect Analysis

This example describes a multi-step delivery of compounds into cells using an electrode array.

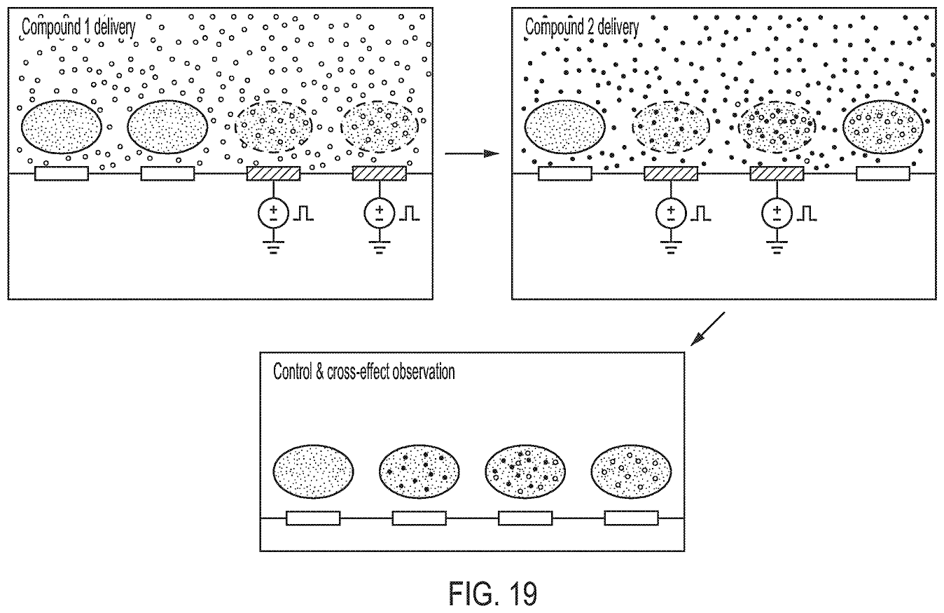

FIG. 19 shows a series of schematic diagrams illustrating a process for a control and cross-effect delivery using spatial addressing and serial delivery via gas generation, according to some embodiments of the present technology. A further advantage of cross-compound effect screening may stem from electrode properties not being appreciably modified during gas evolution, in combination with spatial capabilities of the addressable electrodes of the array. For example, if two compounds are desired to be investigated for their effects on cells, just two compound delivery steps would be needed to form a complete matrix of drug effects.

### Example 13: Extracellular Electrochemical Mapping

This example describes electrochemical mapping using redox electrochemistry on an electrode array.

Electrochemical measurements of cells using electrodes can use a single, large working electrode to measure bulk concentrations of analytes in solution. Such electrochemical electrode-based measurements include the Clark electrode for dissolved oxygen concentration measurement and hydrogen ion concentration (pH) measurements. According to an aspect of the present disclosure, an array of electrochemical electrodes may be used to spatially map analyte concentrations measured via electronics within CMOS integrated circuitry. Such electrochemical mapping may then be applied for cell analysis of cells cultured directly on top of the electrode array.

In this example, to demonstrate the capability for electrochemical mapping using an array of electrodes measured using CMOS integrated circuitry, cyclic voltammetry was performed using a common redox couple of ferricyanide/ferrocyanide, [Fe(CN)₆]³⁻/[Fe(CN)₆]⁴⁻ according to the following expression:

$$\begin{matrix}{{{{[ Fe ( CN )}_{6} ]}^{3 -} + 1 {e}^{-} ⇌ [ Fe ( CN )}_{6} ]}^{4 -} & {E}^{0} = + 0.36 V .\end{matrix} \qquad ( Eq . 7 )$$

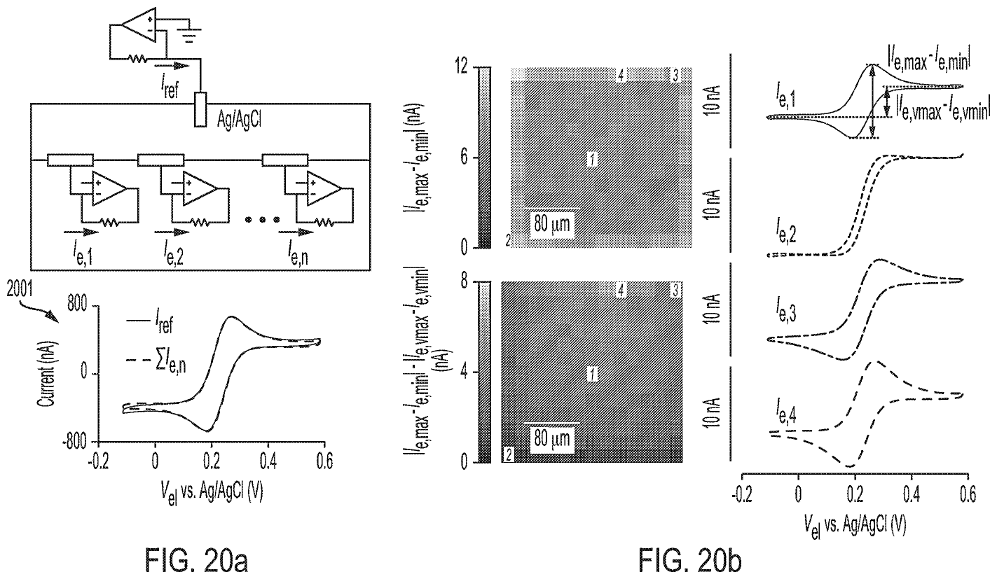

FIG. 20a shows a schematic diagram of a cyclic voltammetry configuration that uses CMOS integrated transimpedance amplifiers (TIAs) to measure each electrode's current and uses an external TIA to measure current through a Ag/AgCl pseudo reference electrode. The configuration may be used with an array of 13×13 Pt electrodes. In an experiment using the configure, a cyclic voltage ramp was applied at a scan rate of 35 mV/s with 1.5 M KCl+5 mM K3[Fe(CN)6] and current was measured. A cyclic-voltammetry plot 2001 of measured current as a function of applied voltage is shown in FIG. 20a, under the schematic diagram. A sum of the currents for the 13×13 electrodes was used for the measurement, which equals that of the reference electrode. FIG. 20b shows two spatial maps: one of a max range of the electrodes' currents (|_{Ie,max}−I_{e,min}|; top left), which relates to diffusion of ferricyanide (starting reactant), and one of a max range of currents minus the max/min voltage currents (|I_{e,max}−I_{e,min}|−|I_{e,vmax}−I_{e,vmin}|; bottom left), which relates to diffusion of ferrocyanide (product). Examples of individual electrode recordings are shown in FIG. 20b, on the right. A non-radial ferrocyanide diffusion is attributed to convection effects in solution.

In this experiment, the array of 13×13=169 electrodes, which is a sub-array or subset of a 64×64 electrode array, was respectively connected to a same number of TIAs and a cyclic linear voltage ramp was applied, as illustrated in FIG. 20a. FIG. 20b shows spatial maps of current density indicating increased cathodic and anodic current magnitudes in electrodes at edge regions of the sub-array, which may be attributed to increased radial diffusion or mass transport at the edge regions in comparison to planar diffusion for electrodes at a central region of the sub-array. Generation of products may limit current density, which can be visualized by a peak current range minus a voltage maximum/minimum current range, as illustrated in the cyclic-voltammetry plot 2001. The cyclic-voltammetry plot 2001 shows a tendency for product diffusion towards the upper right corner. This indicates the capability of spatial measurement of current via current-based electrochemical mapping.

An open-circuit potential of the electrodes may be used to measure a concentration of chemical species in solution. For a high-concentration of a redox couple in solution, the open-circuit potential of platinum electrodes in solution can be determined by the Nernst equation. The Nernst equation relates the reduction potential of an electrochemical reaction to the standard electrode potential, temperature, and activities of the chemical species undergoing reduction and oxidation, according to the following relationships:

$$Ox + {ne}^{-} ⇌ Red \\ {E}_{H} = {E}^{0} - \frac{{φ}_{t}}{n} ln \frac{[ Red ]}{[ Ox ]} , \qquad ( Eq . 8 )$$

where E_{H} is the electrode voltage potential with respect to the standard hydrogen electrode (S.H.E), E⁰ is the half-cell reduction potential, Pt, is the thermal voltage (about 25.7 mV at 25° C.), [Ox]/[Red] is the concentration of the oxidized/reduced chemical species, and n is the number of electrons transferred in the cell half reaction. For a ferricyanide/ferrocyanide reaction, measurement of the open-circuit potential may reflect the ratio of the concentrations of these ions in solution.

In this example, the potential of the remainder of the electrode array was measured. In particular, ferrocyanide generation and transport across an electrode was mapped using a measured open-circuit potential.

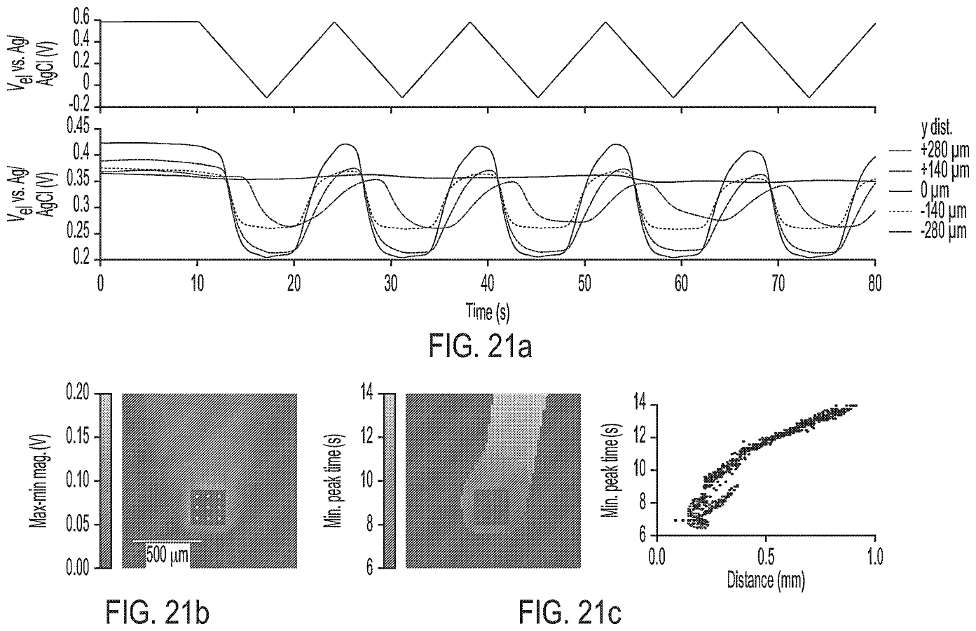

A cyclic potential was applied to a group of 13×13 electrodes, with 9 electrodes excluded within the group of 13×13 electrodes (see FIG. 21b) while open-circuit potentials were measured for the remaining electrodes. FIG. 21a shows data plots of electrode voltages, Vₑₗ, corresponding to open-circuit potentials measured over a period of time. The data plots indicate ferricyanide/ferrocyanide concentration may determine the measured open-circuit potential. That is, whether the open-circuit potential increases or decreases may be affected by a change in ferricyanide/ferrocyanide concentration. FIG. 21b shows a heat map that illustrates, for one cycle, an overall amplitude (maximum minus the minimum) of the open-circuit potential plotted across the array, indicating diffusion or mass transport towards the upper left corner. FIG. 21c shows a heat map and a data plot illustrating a minimum time of the open-circuit potential plotted versus distance from the center of the 13×13 array of electrodes, showing the transient aspects of the diffusion or mass transport.

This example demonstrates, via measurement of open-circuit potentials around an electrode to which cyclic voltammetry is applied, that ferrocyanide may flow during electrochemical reactions, such indicated by the flow of ferrocyanide towards the upper right corner of the array.

### Example 14: Electrochemical Oxygen Mapping of Cells

This example describes a technique that applies electrochemical mapping to cell analysis. For example, a signal from a Clark electrode based on platinum may be measured by applying a pulsed voltage or a voltage pulse sequence, which sequentially oxidizes and then reduces the platinum. As platinum oxide blocks oxygen reduction, the current may drop to zero after the oxide is formed. When the oxide is then reduced, the platinum electrode may pass a negative current due to the presence of oxygen, according to the following expression:

$$\begin{matrix}{O}_{2} ( g ) + 4 {H}^{+} + 4 {e}^{-} ⇌ 2 {H}_{2} O & {E}^{0} = + 1.23 V .\end{matrix} \qquad ( Eq . 9 )$$

A local oxygen concentration is then consumed, and the electrode waits for further diffusion of additional oxygen to the electrode to pass current. Therefore, a rate of Eq. 9 may be limited by oxygen diffusion, which is proportional to the oxygen concentration in solution, and can be measured by measuring current at the electrode.

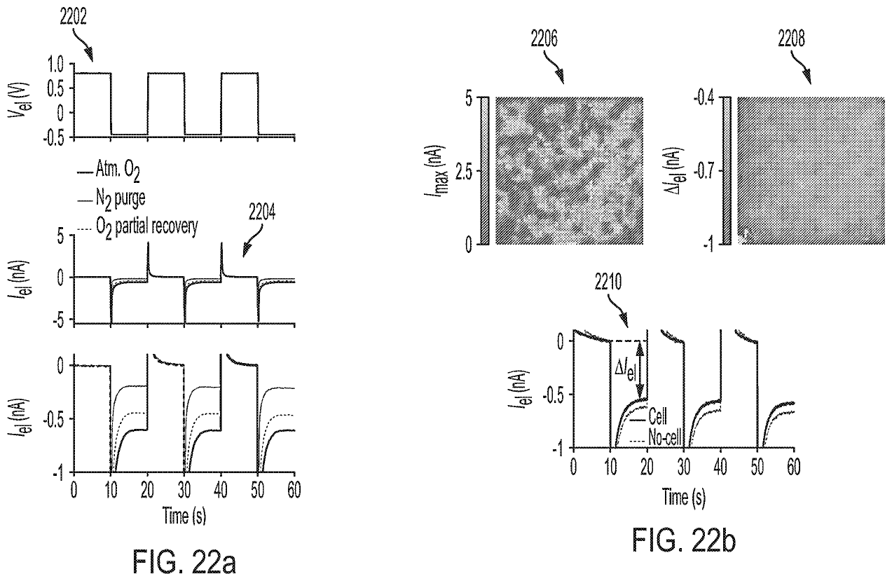

In an experiment using a CMOS electrode array, measurements were performed using a salt solution (phosphate-buffered saline) exposed to ambient air and then subsequently purged with nitrogen gas to reduce the oxygen concentration. FIG. 22a shows a voltage pulse sequence 2202 applied to stimulation electrodes in the electrode array. FIG. 22a also shows a series of data plots 2204 of electrode current, Iₑₗ, as a function of time, measured using the electrode array in ambient air, with a partial nitrogen purge, and a partial recovery (N₂ purge stopped), respectively. The data plots 2204 indicate that electrode current may reflects oxygen concentration. A comparison of the electrode current, Iₑₗ, before and after purging shows a marked reduction.

Experiments were then performed with HEK293 cells, results of which are shown in FIG. 22b. FIG. 22b shows a cross-electrode impedance heat map 2206 using a cross-electrode max current, Iₘₐₓ, over the electrode array area, and also shows a heat map 2208 of a change in electrode current, ΔIₑₗ, over the electrode array area, for HEK293 cells. Included in FIG. 22b is a data plot 2210 of electrode current, Iₑₗ, as a function of time, indicating that for HEK293 cells there is a decrease in oxygen concentration where cells are located, consistent with the cross-electrode impedance map.

Cells consume oxygen as a part of aerobic metabolism, therefore the oxygen concentration around cells may be smaller than in areas without cells. Indeed, mapping electrode current across the array shows that the locations of cells have a smaller magnitude of current than areas without cells, as imaged using a cross-electrode impedance map. The left and bottom edges of the heat map 2208 also show a relatively larger magnitude of current, which is attributed to edge effects and increased diffusion or mass transport.

### Example 15: Effects of Platinum Black and Frequency on Cell Barrier Sensitivity

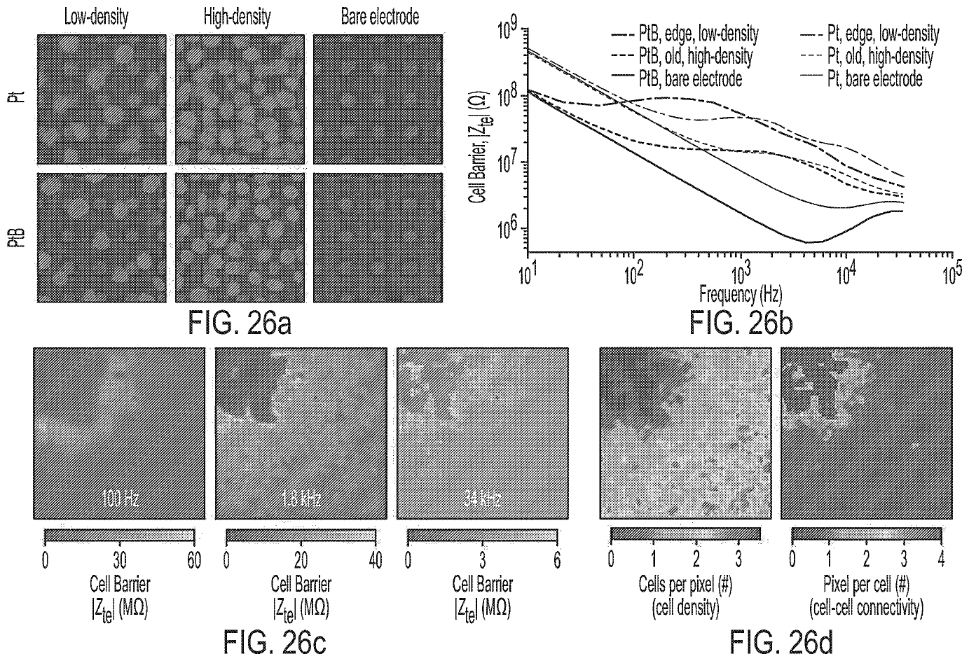

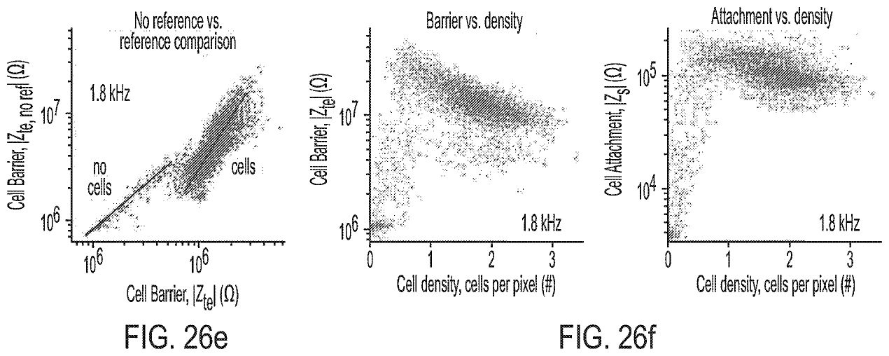

In this example, platinum black (PtB) was used to lower the electrode impedance, Zₑ, to improve cell barrier measurement sensitivity. FIG. 26a shows results of a comparison study of electrode impedance for cells cultured at approximately 72 hours with electrodes under three scenarios: low cell density, high cell density, and without cells. FIG. 26b is a data plot indicating that PtB lowered the Zₜₑ measurement of bare electrodes by about a factor of 5, allowing cell-cell connections of two different densities to be measured with a higher signal-to-noise ratio. FIG. 26c illustrates cell barrier maps versus a reference at different frequencies. Measurements at a low frequency of 100 Hz show more spread and do not capture a sheet edge of the cells, measurements 34 kHz show a relatively higher contrast, and measurements at 1.8 kHz show an even higher contrast for cell-connection measurement when compared to density maps extracted from imaging. FIG. 26d shows cell density and connectivity maps extracted from the nuclei of the fluorescence images. FIG. 26e shows a comparison between Zₜₑ measured without and with a reference at 1.8 kHz. A slightly smaller Zₜₑ was measured without the reference, but for regions with cells and without (the two clusters) the relationship is direct. Measurements without the reference are preferred, as the Zₑ contribution can be easily subtracted from the cell-substrate attachment measurement. FIG. 26f shows a comparison between Zₜₑ and Zₛ versus extracted cell density. For this comparison, Zₛ is down-sampled via a bilinear interpolation to have the same spatial resolution as the Zₜₑ measurement. The cell barrier shows a stronger dependence on cell density due to its measurement geared towards cell-cell connectivity. There's a small correlation between Zs and cell density as well, which can be seen from the circuit model discussed above (see FIG. 4B) as having an effect if Zₛ is high and the assumption that Zₛ<<Zₜₑ no long holds, which was used in the Zₛ calculation.

1. A device for performing an electrical assessment in a biological environment, comprising:
a substrate having an exposed surface comprised of a plurality of holes, at least some of the holes being defined by recessed surfaces coated with a conductive material in electrical contact with electrodes, the electrodes each being in electrical contact with the conductive material of at least one of the holes; and
circuitry controllable to apply one or more stimulus signals to the conductive material of each hole by applying the one or more stimulus signals to the electrode in electrical contact with the hole.

2. The device of claim 1, wherein the exposed surface is configured to be exposed to the biological environment.

3. The device of claim 1, wherein, for each hole of the at least some of the holes, the conductive material defines an interior that is configured to be exposed to the biological environment.

4-10. (canceled)

11. The device of claim 1, wherein one or more holes of the at least some of the holes are in electrical contact with a same electrode.

12-13. (canceled)

14. The device of any one of claims 1 through 13, wherein:
the holes are circular holes, and
the holes include first holes having a first diameter and second holes having a second diameter different from the first diameter.

15. The device of claim 1, wherein each hole of at least some of the holes is disposed in a well structure comprised of a wall surrounding the hole such that the wall surrounds at least one hole of the at least some of the holes, the wall having a height extending above the exposed surface, the height of the wall corresponding to a depth of the well.

16-20. (canceled)

21. A substrate, comprising:
an exposed surface comprised of a plurality of holes, at least some of the holes being defined by recessed surfaces coated with a conductive material; and
integrated circuitry comprised of a plurality of pixel circuits, each of the pixel circuits being in electrical communication with the conductive material of one or more holes of the at least some of the holes.

22. The substrate of claim 21, wherein, for each hole of the at least some of the holes, the hole is defined by at least one conductive sidewall surrounding an interior that is recessed from the exposed surface.

23. The substrate of claim, wherein, for each hole of the at least some of the holes, the hole is further defined by a conductive base portion at a base of the interior.

24. The substrate of claim 21, wherein the at least one conductive sidewall and the conductive base portion are formed of the conductive material.

25. The substrate of claim 21, wherein the interior is configured to be exposed to a biological environment disposed on the exposed surface.

26-40. (canceled)

41. The substrate of claim 21, wherein the insulative material is comprised of a first insulative layer and a second insulative layer.

42. The substrate of claim 21, wherein the electrode pad is disposed in the first insulative layer, and the second insulative layer is disposed between the first insulative layer and the exposed surface.

43. The substrate of claim 21, wherein the exposed surface is a surface of the second insulative layer.

44. The substrate of claim 21, wherein each hole of the one or more holes is comprised of a conductive base portion disposed on the corresponding electrode pad.

45-61. (canceled)

62. A method of manufacturing a semiconductor device on a substrate, comprising:
forming an integrated circuit on the substrate, the integrated circuit being comprised of an electrode array;
forming at least one insulative layer above the electrode array;
forming a plurality of trenches in the at least one insulative layer; and
coating one or more sidewalls of the trenches with a conductive material to form an array of holes in electrical contact with the electrode array.

63. The method of claim 62, wherein, for each hole of the array of holes, the conductive material defines an interior that is recessed from an exposed surface of the substrate and that is exposed to an exterior environment of the semiconductor device.

64. The method of claim 62, wherein the coating of the conductive material is comprised of depositing a layer of the conductive material.

65-66. (canceled)

67. The method of claim 62, further comprising, prior to the forming of the trenches:
forming an array of well structures in the at least one insulative layer, each of the well structures being comprised of a wall height extending above an exposed surface of the substrate, the height of the wall corresponding to a depth of the well.

68. (canceled)

69. The method of claim 62, wherein the forming of the plurality of trenches is comprised of forming at least one trench in each well structure such that the wall of the well structure surrounds the at least one trench.

70-71. (canceled)
